(19) United States
(12) Patent Application Publication
(10) Pub. No.: US 2010/0240604 A1
(43) Pub. Date: Sep. 23, 2010

(54) PROTECTED NUCLEOTIDE ANALOGS

(75) Inventors: Leonid Beigelman, San Mateo, CA (US); Lawrence Blatt, San Francisco, CA (US); Harri Lonnberg, Turku (FI)

Correspondence Address: KNOBBE MARTENS OLSON & BEAR LLP, 2040 MAIN STREET, FOURTEENTH FLOOR, IRVINE, CA, 92614, US

(73) Assignee: Alios BioPharma, Inc., South San Francisco, CA (US)

(21) Appl. No.: 12/728,068

(22) Filed: Mar. 19, 2010

Related U.S. Application Data
(60) Provisional application No. 61/162,171, filed on Mar. 20, 2009.

(51) Int. Cl. A61K 31/7072; C07H 19/056; A61K 31/7056; C07H 19/10; A61K 31/7068; A61P 35/00; A61P 31/12; A61P 35/02; A61P 31/18; A61P 33/00; A61P 31/20; A61P 31/14; A61P 31/22

(52) U.S. Cl. 514/51; 536/26.9; 514/52; 536/26.8; 514/49

(57) ABSTRACT

Disclosed herein are nucleotide analogs with protected phosphates, methods of synthesizing nucleotide analogs with protected phosphates and methods of treating diseases and/or conditions such as viral infections, cancer, and/or parasitic diseases with the nucleotide analogs with protected phosphates.

### BACKGROUND

1. Field

The present application relates to the fields of chemistry, biochemistry and medicine. More particularly, disclosed herein are nucleotide analogs with protected phosphates, pharmaceutical compositions that include one or more nucleotide analogs with protected phosphates and methods of synthesizing the same. Also disclosed herein are methods of treating diseases and/or conditions with the nucleotide analogs with protected phosphates.

2. Description of the Related Art

Nucleoside analogs are a class of compounds that have been shown to exert antiviral and anticancer activity both in vitro and in vivo, and thus, have been the subject of widespread research for the treatment of viral infections and cancer. Nucleoside analogs are therapeutically inactive compounds that are converted by host or viral enzymes to their respective active anti-metabolites, which, in turn, inhibit polymerases involved in viral or cell proliferation. The activation occurs by a variety of mechanisms, such as the addition of one or more phosphate groups and, or in combination with, other metabolic processes.

Nucleoside analogs suffer from several problems that limit their use in treating viral infections and cancer. Nucleoside analogs depend upon intracellular phosphorylation to be biologically active. The absence or low activity of the necessary enzymes for phosphorylation can hamper the conversation of the nucleoside analog to its biologically active form. In addition, nucleoside analogs must be able to penetrate cell membranes and gain access to the intracellular space to be effective as therapeutics. Some nucleoside analogs traverse cell membranes by diffusional processes, which are governed by the charge and lipophilicity of the molecule. Others enter the cell by interaction with transporters for nucleosides present in the cell membrane. However, nucleoside analogs characteristically exhibit poor membrane permeability and are poorly soluble in water, thus, limiting their ability to penetrate cells. Furthermore, when administered to patients, studies have shown that nucleoside analogs are toxic to the liver, bone marrow and nervous system.

Use of nucleotide analogs overcomes the problem of the initial phosphorylation step. Nucleotide analogs are also structurally and metabolically closer to the therapeutically active form. However, the negatively charged phosphate on the nucleotide analogs severely limits the penetration of the nucleotide analogs into the cells. Prior attempts to neutralize the charge on the phosphate have resulted in nucleotide analogs with poor plasma stability, insufficient intracellular lability (releasability) and/or poor therapeutic efficacy.

### SUMMARY

An embodiment disclosed herein relates to a compound of Formula (I), or a pharmaceutically acceptable salt, prodrug or prodrug ester thereof.

Another embodiment disclosed herein relates to a compound of Formula (II), or a pharmaceutically acceptable salt, prodrug or prodrug ester thereof.

Still an embodiment disclosed herein relates to a compound of Formula (III), or a pharmaceutically acceptable salt, prodrug or prodrug ester thereof. An embodiment disclosed herein relates to a compound of Formula (III), or a pharmaceutically acceptable salt, prodrug or prodrug ester thereof, with a nucleoside portion having the structure of Formula (IV). Another embodiment disclosed herein relates to a compound of Formula (III), or a pharmaceutically acceptable salt, prodrug or prodrug ester thereof, with a nucleoside portion having the structure of Formula (V).

Yet still another embodiment disclosed herein relates to thymidine 5′-bis[3-acetyloxymethoxy-2,2-bis(ethoxycarbonyl)propyl]phosphate and thymidine 5′-bis[3-acetyloxy-2,2-bis(ethoxycarbonyl)propyl]phosphate.

Some embodiments disclosed herein relate to methods of synthesizing a compound of Formula (I).

Other embodiments disclosed herein relate to methods of synthesizing a compound of Formula (II).

Still other embodiments disclosed herein relate to methods of synthesizing a compound of Formula (III).

An embodiment disclosed herein relates to pharmaceutical compositions that can include one or more compounds of Formulae (I), (II) and (III), or a pharmaceutically acceptable carrier, diluent, excipient or combination thereof. The pharmaceutical compositions of the compounds of Formula (I), (II) and (III) can be used in the manufacture of a medicament for treating an individual suffering from a neoplastic disease, a viral infection, or a parasitic disease. The pharmaceutical compositions of the compounds of Formula (I), (II) and (III) can be used for treating a neoplastic disease, a viral infection, or a parasitic disease.

Some embodiments disclosed herein relate to methods of ameliorating or treating a neoplastic disease that can include administering to a subject suffering from the neoplastic disease a therapeutically effective amount of one or more compounds of Formulae (I), (II) and (III), or a pharmaceutical composition that includes one or more compounds of Formulae (I), (II) and (III). The compounds of Formula (I), (II) and (III) can be used in the manufacture of a medicament for treating an individual suffering from a neoplastic disease. The compounds of Formula (I), (II) and (III) can be used for treating a neoplastic disease.

Other embodiments disclosed herein relate to methods of inhibiting the growth of a tumor that can include administering to a subject having a tumor a therapeutically effective amount of one or more compounds of Formulae (I), (II) and (III), or a pharmaceutical composition that includes one or more compounds of Formulae (I), (II) and (III).

Still other embodiments disclosed herein relate to methods of ameliorating or treating a viral infection that can include administering to a subject suffering from the viral infection a therapeutically effective amount of one or more compounds of Formulae (I), (II) and (III), or a pharmaceutical composition that includes one or more compounds of Formulae (I), (II) and (III). The compounds of Formula (I), (II) and (III) can be used in the manufacture of a medicament for treating an individual suffering from a viral infection. The compounds of Formula (I), (II) and (III) can be used for treating a viral infection.

Yet still other embodiments disclosed herein relate to methods of ameliorating or treating a parasitic disease that can include administering to a subject suffering from the parasitic disease a therapeutically effective amount of one or more compounds of Formulae (I), (II) and (III), or a pharmaceutical composition that includes one or more compounds of Formulae (I), (II) and (III). The compounds of Formula (I), (II) and (III) can be used in the manufacture of a medicament for treating an individual suffering from a parasitic disease. The compounds of Formula (I), (II) and (III) can be used for treating a parasitic disease.

### DETAILED DESCRIPTION

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, any “R” group(s) such as, without limitation, R¹, R^{1a} and R^{1b}, represent substituents that can be attached to the indicated atom. A non-limiting list of R groups include, but are not limited to, hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. An R group may be substituted or unsubstituted. If two “R” groups are covalently bonded to the same atom or to adjacent atoms, then they may be “taken together” as defined herein to form a cycloalkyl, cycloalkenyl, cycloalkynyl, aryl, heteroaryl or heteroalicyclyl group. For example, without limitation, if Rₐ and R_{b} of an NRₐR_{b} group are indicated to be “taken together”, it means that they are covalently bonded to one another at their terminal atoms to form a ring that includes the nitrogen:

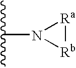

Whenever a group is described as being “optionally substituted” that group may be unsubstituted or substituted with one or more of the indicated substituents. Likewise, when a group is described as being “unsubstituted or substituted” if substituted, the substituent may be selected from one or more the indicated substituents.

The term “substituted” has its ordinary meaning, as found in numerous contemporary patents from the related art. See, for example, U.S. Pat. Nos. 6,509,331; 6,506,787; 6,500,825; 5,922,683; 5,886,210; 5,874,443; and 6,350,759; all of which are incorporated herein by reference for the limited purpose of disclosing suitable substituents that can be on a substituted group and standard definitions for the term “substituted.” Examples of suitable substituents include but are not limited to hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. Each of these substituents can be further substituted. The other above-listed patents also provide standard definitions for the term “substituted” that are well-understood by those of skill in the art.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl, aryl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

The alkyl group may be substituted or unsubstituted. When substituted, the substituent group(s) is(are) one or more group(s) individually and independently selected from alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof.

As used herein, “alkenyl” refers to an alkyl group that contains in the straight or branched hydrocarbon chain one or more double bonds. An alkenyl group may be unsubstituted or substituted. When substituted, the substituent(s) may be selected from the same groups disclosed above with regard to alkyl group substitution unless otherwise indicated.

As used herein, “alkynyl” refers to an alkyl group that contains in the straight or branched hydrocarbon chain one or more triple bonds. An alkynyl group may be unsubstituted or substituted. When substituted, the substituent(s) may be selected from the same groups disclosed above with regard to alkyl group substitution unless otherwise indicated.

As used herein, “aryl” refers to a carbocyclic (all carbon) monocyclic or multicyclic aromatic ring system that has a fully delocalized pi-electron system. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof, unless the substituent groups are otherwise indicated.

As used herein, “heteroaryl” refers to a monocyclic or multicyclic aromatic ring system (a ring system with fully delocalized pi-electron system) that contain(s) one or more heteroatoms, that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. A heteroaryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof.

An “aralkyl” is an aryl group connected, as a substituent, via a lower alkylene group. The lower alkylene and aryl group of an aralkyl may be substituted or unsubstituted. Examples include but are not limited to benzyl, substituted benzyl, 2-phenylalkyl, 3-phenylalkyl, and naphtylalkyl.

A “heteroaralkyl” is heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, and imidazolylalkyl, and their substituted as well as benzo-fused analogs.

The term “lower alkylene groups” are straight-chained tethering saturated hydrocarbon groups, forming bonds to connect molecular fragments via their terminal carbon atoms. Examples include but are not limited to methylene (—CH₂—), ethylene (—CH₂CH₂—), propylene (—CH₂CH₂CH₂—), and butylene (—(CH₂)₄—) groups. A lower alkylene group may be substituted or unsubstituted.

As used herein, “cycloalkyl” refers to a completely saturated (no double or triple bonds) mono- or multi-cyclic hydrocarbon ring system. When composed of two or more rings, the rings may be joined together in a fused, bridged or spiro-connected fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, and the like. If substituted, the substituent(s) may be selected from those substituents indicated above with respect to substitution of an aryl group unless otherwise indicated.

As used herein, “cycloalkenyl” refers to a cycloalkyl group that contains one or more double bonds in the ring; although, if there is more than one, the double bonds cannot form a fully delocalized pi-electron system (otherwise the group would be “aryl,” as defined herein). When composed of two or more rings, the rings may be connected together in a fused, bridged or spiro-connected fashion. A cycloalkenyl group may be unsubstituted or substituted. When substituted, the substituent(s) may be selected from the substituents disclosed above with respect to an aryl group substitution unless otherwise indicated.

As used herein, “cycloalkynyl” refers to a cycloalkyl group that contains one or more triple bonds in the ring. If there is more than one triple bond, the triple bonds cannot form a fully delocalized pi-electron system. When composed of two or more rings, the rings may be joined together in a fused, bridged or spiro-connected fashion. A cycloalkynyl group may be unsubstituted or substituted. When substituted, the substituent(s) may be selected from the substituents disclosed above with respect to an aryl group substitution unless otherwise indicated.

As used herein, “heteroalicyclic” or “heteroalicyclyl” refers to a stable 3- to 18 membered monocyclic, bicyclic, tricyclic, or tetracyclic ring system which consists of carbon atoms and from one to five heteroatoms such as nitrogen, oxygen and sulfur. The “heteroalicyclic” or “heteroalicyclyl” may be joined together in a fused, bridged or spiro-connected fashion; and the nitrogen, carbon and sulfur atoms in the “heteroalicyclic” or “heteroalicyclyl” may be optionally oxidized; the nitrogen may be optionally quaternized; and the rings may also contain one or more double bonds provided that they do not form a fully delocalized pi-electron system throughout all the rings. Heteroalicyclyl or heteroalicyclic groups may be unsubstituted or substituted. When substituted, the substituent(s) may be one or more groups independently selected from: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, haloalkyl, haloalkoxy, trihalomethanesulfonyl, trihalomethanesulfonamido, and amino, including mono- and di-substituted amino groups, and the protected derivatives thereof. Examples of such “heteroalicyclic” or “heteroalicyclyl” groups include but are not limited to, azepinyl, acridinyl, carbazolyl, cinnolinyl, 1,3-dioxin, 1,3-dioxane, 1,4-dioxane, 1,2-dioxolanyl, 1,3-dioxolanyl, 1,4-dioxolanyl, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazolinyl, imidazolidine, isoxazoline, isoxazolidine, oxazoline, oxazolidine, oxazolidinone, thiazoline, thiazolidine, morpholinyl, oxiranyl, piperidinyl N-Oxide, piperidinyl, piperazinyl, pyrrolidinyl, pyrrolidone, pyrrolidione, 4-piperidonyl, pyrazoline, pyrazolidinyl, 2-oxopyrrolidinyl, tetrahydropyran, 4H-pyran, tetrahydrothiopyran, thiamorpholinyl, thiamorpholinyl sulfoxide, thiamorpholinyl sulfone, and their benzo-fused analogs (e.g., benzimidazolidinone, tetrahydroquinoline, 3,4-methylenedioxyphenyl).

A “(heteroalicyclyl)alkyl” is a heterocyclic or a heteroalicyclylic group connected, as a substituent, via a lower alkylene group. The lower alkylene and heterocyclic or a heterocyclyl of a (heteroalicyclyl)alkyl may be substituted or unsubstituted. Examples include but are not limited tetrahydro-2H-pyran-4-yl)methyl, (piperidin-4-yl)ethyl, (piperidin-4-yl)propyl, (tetrahydro-2H-thiopyran-4-yl)methyl, and (1,3-thiazinan-4-yl)methyl.

As used herein, “alkoxy” refers to the formula —OR wherein R is an alkyl is defined as above, e.g. methoxy, ethoxy, n-propoxy, 1-methylethoxy (isopropoxy), n-butoxy, iso-butoxy, sec-butoxy, tert-butoxy, and the like. An alkoxy may be substituted or unsubstituted.

As used herein, “acyl” refers to a hydrogen, alkyl, alkenyl, alkynyl, or aryl connected, as substituents, via a carbonyl group. Examples include formyl, acetyl, propanoyl, benzoyl, and acryl. An acyl may be substituted or unsubstituted.

As used herein, “hydroxyalkyl” refers to an alkyl group in which one or more of the hydrogen atoms are replaced by hydroxy group. Examples of hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

As used herein, “haloalkyl” refers to an alkyl group in which one or more of the hydrogen atoms are replaced by halogen (e.g., mono-haloalkyl, di-haloalkyl and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by halogen (e.g., mono-haloalkoxy, di-haloalkoxy and tri-haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

As used herein, “aryloxy” and “arylthio” refers to RO— and RS—, in which R is an aryl, such as but not limited to phenyl. Both an aryloxy and arylthio may be substituted or unsubstituted.

A “sulfenyl” group refers to an “—SR” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O—” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂-” group wherein X is a halogen.

A “trihalomethanesulfonamido” group refers to an “X₃CS(O)₂R_{A}N—” group wherein X is a halogen and R_{A} defined with respect to O-carboxy.

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “mercapto” group refers to an “—SH” group.

A “carbonyl” group refers to a C═O group.

An “S-sulfonamido” group refers to a “—SO₂NR_{A}R_{B}” group in which R_{A} and R_{B} can be the same as R defined with respect to O-carboxy. An S-sulfonamido may be substituted or unsubstituted.

An “N-sulfonamido” group refers to a “RSO₂N(R_{A})—” group in which R and R_{A} can be the same as R defined with respect to O-carboxy. A N-sulfonamido may be substituted or unsubstituted.

An “O-carbamyl” group refers to a “—OC(═O)NR_{A}R_{B}” group in which R_{A} and R_{B} can be the same as R defined with respect to O-carboxy. An O-carbamyl may be substituted or unsubstituted.

An “N-carbamyl” group refers to an “ROC(═O)NR_{A}—” group in which R and R_{A} can be the same as R defined with respect to O-carboxy. An N-carbamyl may be substituted or unsubstituted.

An “O-thiocarbamyl” group refers to a “—OC(═S)—NR_{A}R_{B}” group in which R_{A} and R_{B} can be the same as R defined with respect to O-carboxy. An O-thiocarbamyl may be substituted or unsubstituted.

An “N-thiocarbamyl” group refers to an “ROC(═S)NR_{A}-” group in which R and R_{A} can be the same as R defined with respect to O-carboxy. An N-thiocarbamyl may be substituted or unsubstituted.

A “C-amido” group refers to a “—C(═O)NR_{A}R_{B}” group in which R_{A} and R_{B} can be the same as R defined with respect to O-carboxy. A C-amido may be substituted or unsubstituted.

An “N-amido” group refers to a “RC(═O)NR_{A}—” group in which R and R_{A} can be the same as R defined with respect to O-carboxy. An N-amido may be substituted or unsubstituted.

As used herein, “organylcarbonyl” refers to a group of the formula —C(═O)Rₐ wherein Rₐ can be alkyl, alkenyl, alkynyl, cycloalkyl, cycloalkenyl, cycloalkynyl, aryl, heteroaryl, heteroalicyclyl, aralkyl, or (heteroalicyclyl)alkyl. An organylcarbonyl can be substituted or unsubstituted.

The term “alkoxycarbonyl” as used herein refers to a group of the formula —C(═O)ORₐ wherein Rₐ can be the same as defined with respect to organylcarbonyl. An alkoxycarbonyl can be substituted or unsubstituted.

As used herein, “organylaminocarbonyl” refers to a group of the formula —C(═O)NRₐR_{b} wherein Rₐ and R_{b} can each be independently selected from the same substituents as defined with respect to organylcarbonyl. An organylaminocarbonyl can be substituted or unsubstituted.

As used herein, the term “levulinoyl” refers to a C(═O)CH₂CH₂C(═O)CH₃ group.

The term “halogen atom,” as used herein, means any one of the radio-stable atoms of column 7 of the Periodic Table of the Elements, i.e., fluorine, chlorine, bromine, or iodine, with bromine and chlorine being preferred.

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the term “nucleoside” refers to a compound composed of any pentose or modified pentose moiety attached to a specific portion of a heterocyclic base, tautomer, or derivative thereof such as the 9-position of a purine, 1-position of a pyrimidine, or an equivalent position of a heterocyclic base derivative. Examples include, but are not limited to, a ribonucleoside comprising a ribose moiety and a deoxyribonucleoside comprising a deoxyribose moiety. In some instances, the nucleoside can be a nucleoside drug analog.

As used herein, the term “nucleoside drug analog” refers to a compound composed of a nucleoside that has therapeutic activity, such as antiviral, anti-neoplastic, anti-parasitic and/or antibacterial activity.

As used herein, the term “nucleotide” refers to a nucleoside having a phosphate ester substituted on the 5′-position or an equivalent position of a nucleoside derivative.

As used herein, the term “heterocyclic base” refers to a purine, a pyrimidine and derivatives thereof. The term “purine” refers to a substituted purine, its tautomers and analogs thereof. Similarly, the term “pyrimidine” refers to a substituted pyrimidine, its tautomers and analogs thereof. Examples of purines include, but are not limited to, purine, adenine, guanine, hypoxanthine, xanthine, theobromine, caffeine, uric acid and isoguanine. Examples of pyrimidines include, but are not limited to, cytosine, thymine, uracil, and derivatives thereof. An example of an analog of a purine is 1,2,4-triazole-3-carboxamide.

Other non-limiting examples of heterocyclic bases include diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-fluorouracil, 5-bromouracil, pseudoisocytosine, isocytosine, isoguanine, and other heterocyclic bases described in U.S. Pat. Nos. 5,432,272 and 7,125,855, which are incorporated herein by reference for the limited purpose of disclosing additional heterocyclic bases.

As used herein, the term “protected heterocyclic base” refers to a heterocyclic base in which one or more amino groups attached to the base are protected with one or more suitable protecting groups and/or one or more —NH groups present in a ring of the heterocyclic base are protected with one or more suitable protecting groups. When more than one protecting group is present, the protecting groups can be the same or different.

The term “—O-linked amino acid” refers to an amino acid that is attached to the indicated moiety via its main-chain carboxyl function group. When the amino acid is attached, the hydrogen that is part of the —OH portion of the carboxyl function group is not present and the amino acid is attached via the remaining oxygen. The term “—N-linked amino acid” refers to an amino acid that is attached to the indicated moiety via its main-chain amino or mono-substituted amino group. As used herein, the term “amino acid” refers to any amino acid (both standard and non-standard amino acids), including, but limited to, α-amino acids β-amino acids, γ-amino acids and δ-amino acids. Examples of suitable amino acids, include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine.

The terms “derivative,” “variant,” or other similar terms refer to a compound that is an analog of the other compound.

The terms “protecting group” and “protecting groups” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls (e.g., t-butoxycarbonyl (BOC)); arylalkylcarbonyls (e.g., benzyloxycarbonyl, benzoyl); substituted methyl ether (e.g. methoxymethyl ether); substituted ethyl ether; a substituted benzyl ether; tetrahydropyranyl ether; silyl ethers (e.g., trimethylsilyl, triethylsilyl, triisopropylsilyl, t-butyldimethylsilyl, or t-butyldiphenylsilyl); esters (e.g. benzoate ester); carbonates (e.g. methoxymethylcarbonate); sulfonates (e.g. tosylate, mesylate); acyclic ketal (e.g. dimethyl acetal); cyclic ketals (e.g., 1,3-dioxane or 1,3-dioxolanes); acyclic acetal; cyclic acetal; acyclic hemiacetal; cyclic hemiacetal; and cyclic dithioketals (e.g., 1,3-dithiane or 1,3-dithiolane).

“Leaving group” as used herein refers to any atom or moiety that is capable of being displaced by another atom or moiety in a chemical reaction. More specifically, in some embodiments, “leaving group” refers to the atom or moiety that is displaced in a nucleophilic substitution reaction. In some embodiments, “leaving groups” are any atoms or moieties that are conjugate bases of strong acids. Examples of suitable leaving groups include, but are not limited to, tosylates and halogens. Non-limiting characteristics and examples of leaving groups can be found, for example in Organic Chemistry, 2d ed., Francis Carey (1992), pages 328-331; Introduction to Organic Chemistry, 2d ed., Andrew Streitwieser and Clayton Heathcock (1981), pages 169-171; and Organic Chemistry, 5^{th} ed., John McMurry (2000), pages 398 and 408; all of which are incorporated herein by reference for the limited purpose of disclosing characteristics and examples of leaving groups.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference for the limited purpose describing procedures and preparation of suitable prodrug derivatives.

The term “pro-drug ester” refers to derivatives of the compounds disclosed herein formed by the addition of any of several ester-forming groups that are hydrolyzed under physiological conditions. Examples of pro-drug ester groups include pivaloyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference for the limited purpose of disclosing ester-forming groups that can form prodrug esters.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine, lysine, and the like.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures. In addition it is understood that, in any compound described herein having one or more double bond(s) generating geometrical isomers that can be defined as E or Z, each double bond may independently be E or Z a mixture thereof. Likewise, all tautomeric forms are also intended to be included.

An embodiment disclosed herein relates to a compound of Formula (I), or a pharmaceutically acceptable salt, prodrug or prodrug ester thereof:

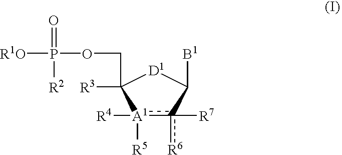

wherein: each [symbol] can be a double or single bond; A¹ can be selected from C (carbon), O (oxygen) and S (sulfur); B¹ can be an optionally substituted heterocyclic base or an optionally substituted heterocyclic base derivative thereof; D¹ can be selected from C═CH₂, CH₂, O (oxygen) and S (sulfur); R¹ can be

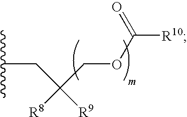

R² can be an —N-linked amino acid; R³ selected from hydrogen, azido, —CN, an optionally substituted C₁₋₄ alkyl and an optionally substituted C₁₋₄ alkoxy; R⁴ can be absent or selected from hydrogen, halogen, hydroxy and an optionally substituted C₁₋₄ alkyl; R⁵ can be absent or selected from hydrogen, halogen, azido, amino, hydroxy and an —O-linked amino acid; R⁶ can be selected from hydrogen, halogen, hydroxy, —CN, —NC, an optionally substituted C₁₋₄ alkyl, an optionally substituted C₁₋₄ alkoxy and an —O-linked amino acid; R⁷ can be absent or selected from hydrogen, halogen, hydroxy, —CN, —NC, an optionally substituted C₁₋₄ alkyl, an optionally substituted haloalkyl and an optionally substituted hydroxyalkyl, or when the bond to R⁶ indicated by [symbol] is a double bond, then R⁶ is a C₁₋₄ alkenyl and R⁷ is absent; R⁸ and R⁹ can be each independently —C≡N or an optionally substituted substituent selected from C₁₋₈ organylcarbonyl, C₁₋₈ alkoxycarbonyl and C₁₋₈ organylaminocarbonyl; R¹⁰ can be hydrogen or an optionally substituted C₁₋₄-alkyl; and m can be 1 or 2.

In an embodiment, m can be 1. In another embodiment, m can be 2. In some embodiments, A¹ can be carbon. In an embodiment, [symbol] can be a single bond. In an embodiment, A¹ can be carbon, D¹ can be oxygen and [symbol] can be a single bond. In some embodiments, A¹ can be carbon, D¹ can be oxygen, [symbol] can be a single bond and m can be 1. In other embodiments, A¹ can be carbon, D¹ can be oxygen, [symbol] can be a single bond and m can be 2.

In some embodiments, the optionally substituted C₁₋₄ alkyl can be selected from methyl, ethyl, n-propyl, isopropyl, n-butyl, iso-butyl and tert-butyl. In some embodiments, the optionally substituted C₁₋₄ alkoxy can be selected from methoxy, ethoxy, n-propoxy, isopropoxy, n-butoxy, iso-butoxy and tert-butoxy.

The substitutents on

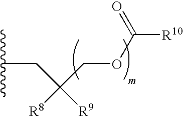

can vary. In some embodiments, R⁸ can be —C≡N and R⁹ can be an optionally substituted C₁₋₈ alkoxycarbonyl such as —C(═O)OCH₃. In other embodiments, R⁸ can be —C≡N and R⁹ can be an optionally substituted C₁₋₈ organylaminocarbonyl, for example, —C(═O)NHCH₂CH₃ and —C(═O)NHCH₂CH₂phenyl. In still other embodiments, both R⁸ and R⁹ can be an optionally substituted C₁₋₈ organylcarbonyl. In an embodiment, both R⁸ and R⁹ can be —C(═O)CH₃. In yet still other embodiments, both R⁸ and R⁹ can be an optionally substituted C₁₋₈ alkoxycarbonyl. In an embodiment, both R⁸ and R⁹ can be —C(═O)OCH₂CH₃. In an embodiment, both R⁸ and R⁹ can be —C(═O)OCH₃. In some embodiments, including those in this paragraph, R¹⁰ can be an optionally substituted C₁₋₄-alkyl. In an embodiment, including those in this paragraph, R¹⁰ can be methyl or tert-butyl.

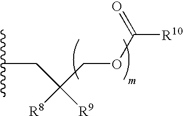

Examples of suitable groups, include but are not limited to, the following:

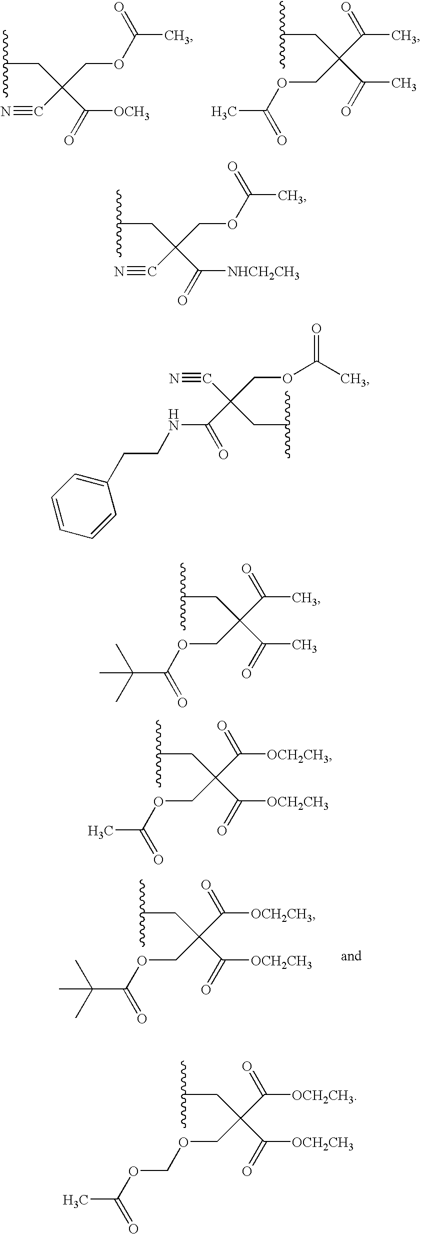

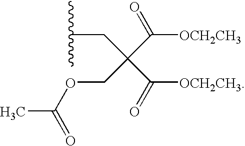

In an embodiment, R¹ can be In another embodiment, R¹ can be

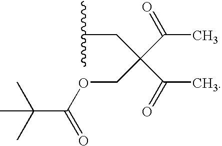

In still another embodiment, R¹ can be

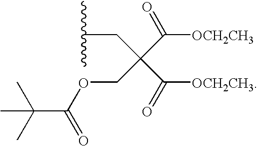

In yet still another embodiment, R¹ can be

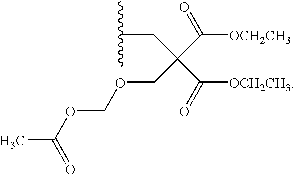

In an embodiment, R¹ can be

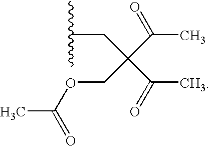

The substituent B¹ can also vary. In some embodiments, B can be selected from:

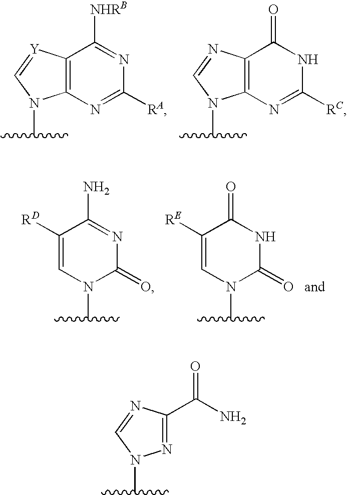

wherein: R^{A} can be hydrogen or halogen; R^{B} can be hydrogen, an optionally substituted C₁₋₄alkyl, or an optionally substituted C₃₋₈ cycloalkyl; R^{C} can be hydrogen or amino; R^{D} can be hydrogen or halogen; R^{E} can be hydrogen or an optionally substituted C₁₋₄alkyl; and Y can be N (nitrogen) or CR^{F}, wherein R^{F} can be selected from hydrogen, halogen, an optionally substituted C₁₋₄-alkyl, an optionally substituted C₂₋₄-alkenyl and an optionally substituted C₂₋₄-alkynyl. In some embodiments, B¹ can be

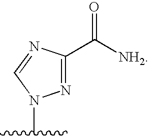

In other embodiments, B¹ can be

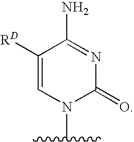

In yet other embodiments, B¹ can be

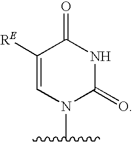

In yet still other embodiments, B¹ can be

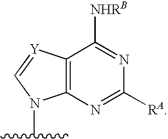

In an embodiment Y can be nitrogen; R^{A} can be hydrogen and R^{B} can be hydrogen. In another embodiment, Y can be CR^{F}, wherein R^{F} can be selected from hydrogen, halogen, an optionally substituted C₁₋₄-alkyl, an optionally substituted C₂₋₄-alkenyl and an optionally substituted C₂₋₄-alkynyl; R^{A} can be hydrogen and R^{B} can be hydrogen. When B¹ is any of the aforementioned moieties shown above, in some embodiments, A¹ can be carbon. In an embodiment, B¹ can be any of the aforementioned moieties shown above, A¹ can be carbon and D¹ can be oxygen. In some embodiments, B¹ can be any of the aforementioned moieties shown above, A¹ can be carbon, D¹ can be oxygen and [symbol] can be a single bond.

Various amino acids can be utilized for the substituent R². In some embodiments, R² can have the structure

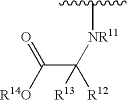

wherein: R¹¹ can be hydrogen or an optionally substituted C₁₋₄-alkyl; R¹² can be selected from hydrogen, an optionally substituted C₁₋₆-alkyl, an optionally substituted aryl, an optionally substituted aryl(C₁₋₄ alkyl) and haloalkyl; R¹³ can be hydrogen or an optionally substituted C₁₋₄-alkyl; and R¹⁴ can be selected from an optionally substituted C₁₋₆ alkyl, an optionally substituted C₆ aryl, an optionally substituted C₁₀ aryl, and an optionally substituted C₃₋₆ cycloalkyl. In an embodiment, R¹¹ can be hydrogen. In some embodiments, R¹² can be an optionally substituted C₁₋₄-alkyl, such as methyl. In an embodiment, R¹³ can be hydrogen or an optionally substituted C₁₋₄-alkyl. In some embodiment, R¹⁴ can be an optionally substituted C₁₋₄-alkyl (e.g., methyl). One example of a suitable R² group includes, but are not limited to,

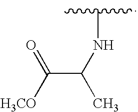

In some embodiments, the amino acid can be in the L-configuration. In other embodiments, the amino acid can be in the D-configuration. For example, R² can be

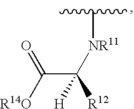

such as

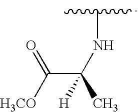

Additional suitable amino acids that can be used in embodiments disclosed herein are described in Cahard et al., Mini-Reviews in Medicinal Chemistry, 2004, 4 371-381 and McGuigan et al., J. Med. Chem., 2008, 51(18) 5807-5812, which hereby incorporated by reference for the limited purpose of describing additional suitable amino acids.

In some embodiments, R⁵ can be hydroxy. In other embodiments, R⁵ can be an —O-linked amino acid. In some embodiments, R⁶ can be hydroxy. In other embodiments, R⁶ can be a C₁₋₄ alkoxy such as methoxy. In still other embodiments, R⁶ can be an —O-linked amino acid. In some embodiments, both R⁵ and R⁶ can be hydroxy groups. In other embodiments, R⁵ can be a hydroxyl group and R⁶ can be —O-linked amino acid. A non-limiting list of suitable —O-linked amino acid include, but are not limited to the following: alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. In an embodiment, the —O-linked amino acid can be valine. In some embodiments, the —O-linked amino acid can be selected from —O-linked α-amino acid, —O-linked β-amino acid, —O-linked γ-amino acid and —O-linked δ-amino acid. In an embodiment, the —O-linked amino acid can be in the L-configuration.

In some embodiments, the compound of Formula (I) can be an anti-neoplastic agent. In other embodiments, the compound of Formula (I) can be an anti-viral agent. In still other embodiments, the compound of Formula (I) can be an anti-parasitic agent.

An embodiment disclosed herein relates to a compound of Formula (II), or a pharmaceutically acceptable salt, prodrug or prodrug ester thereof:

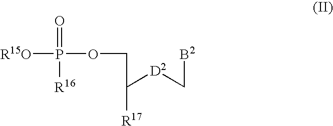

wherein: B² can be an optionally substituted heterocyclic base or an optionally substituted heterocyclic base derivative thereof; D² can be selected from C═CH₂, CH₂, O (oxygen) and S (sulfur); R¹⁵ can be

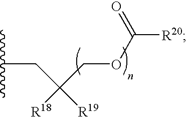

R¹⁶ can be an —N-linked amino acid; R¹⁷ can be hydrogen or —(CH₂)—OH; R¹⁸ and R¹⁹ can be each independently —C≡N or an optionally substituted substituent selected from C₁₋₈ organylcarbonyl, C₁₋₈ alkoxycarbonyl and C₁₋₈ organylaminocarbonyl; R²⁰ can be hydrogen or an optionally substituted C₁₋₄-alkyl; and n can be 1 or 2. In some embodiments, D² can be oxygen. In an embodiment, D² can be oxygen and n can be 1. In another embodiment, D² can be oxygen and n can be 2.

Example of R¹⁵ groups, include but are not limited to the following:

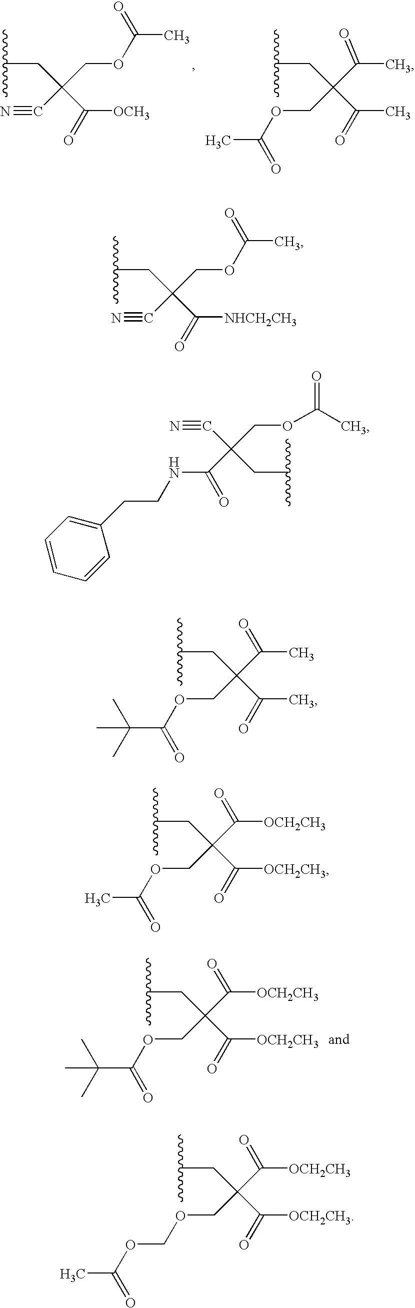

In an embodiment, R¹⁵ can be

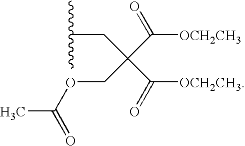

In another embodiment, R¹⁵ can be

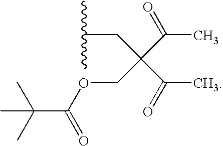

In still another embodiment, R¹⁵ can be

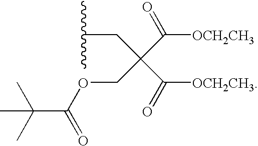

In yet still another embodiment, R¹⁵ can be

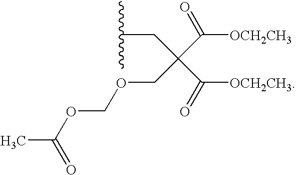

In some embodiments, R¹⁵ can be

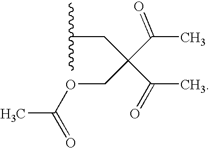

As with R², R¹⁶ can be any suitable amino acid such as those described herein. In some embodiments, R¹⁶ can have the structure

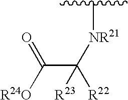

wherein: R²¹ can be hydrogen or an optionally substituted C₁₋₄-alkyl; R²² can be selected from hydrogen, an optionally substituted C₁₋₆-alkyl, an optionally substituted aryl, an optionally substituted aryl(C₁₋₄ alkyl) and haloalkyl; R²³ can be hydrogen or an optionally substituted C₁₋₄-alkyl; and R²⁴ can be selected from an optionally substituted C₁₋₆ alkyl, an optionally substituted C₆ aryl, an optionally substituted C₁₀ aryl, and an optionally substituted C₃₋₆ cycloalkyl. In an embodiment, R²¹ can be hydrogen. In some embodiments, R²² can be an optionally substituted C₁₋₄-alkyl such as methyl. In an embodiment, R²³ can be hydrogen or an optionally substituted C₁₋₄-alkyl (e.g., methyl). In some embodiment, R²⁴ can be an optionally substituted C₁₋₄-alkyl. One example of a suitable R¹⁶ group includes, but are not limited to,

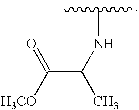

In some embodiments, the amino acid can be in the L-configuration. In other embodiments, the amino acid can be in the D-configuration. For example, R¹⁶ can be

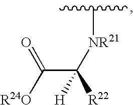

such as

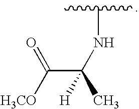

Various optionally substituted heterocyclic bases and optionally substituted heterocyclic base derivatives can be present in a compound of Formula (II). Examples of suitable optionally substituted heterocyclic bases and optionally substituted heterocyclic base derivatives are shown below.

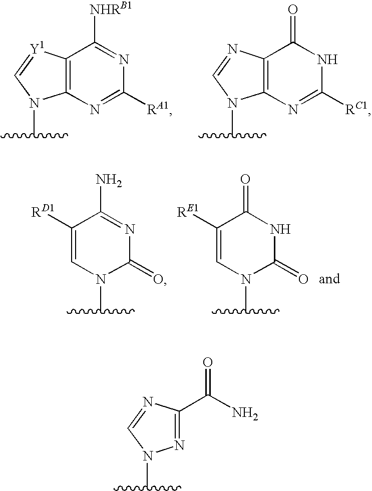

wherein: R^{A1} can be hydrogen or halogen; R^{B1} can be hydrogen, an optionally substituted C₁₋₄alkyl, or an optionally substituted C₃₋₈ cycloalkyl; R^{C1} can be hydrogen or amino; R^{D1} can be hydrogen or halogen; R^{E1} can be hydrogen or an optionally substituted C₁₋₄alkyl; and Y¹ can be N (nitrogen) or CR^{F1}, wherein R^{F1} can be selected from hydrogen, halogen, an optionally substituted C₁₋₄-alkyl, an optionally substituted C₂₋₄-alkenyl and an optionally substituted C₂₋₄-alkynyl. In some embodiments, B² can be

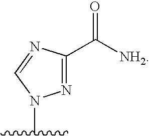

In other embodiments, B² can be

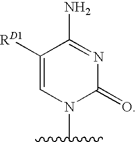

In yet other embodiments, B² can be

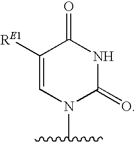

In yet still other embodiments, B² can be

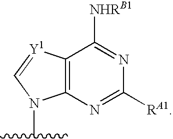

In an embodiment Y¹ can be nitrogen; R^{A1} can be hydrogen and R^{B1} can be hydrogen. In another embodiment, Y¹ can be CR^{F1}, wherein R^{F1} can be selected from hydrogen, halogen, an optionally substituted C₁₋₄-alkyl, an optionally substituted C₂₋₄-alkenyl and an optionally substituted C₂₋₄-alkynyl; R^{A1} can be hydrogen and R^{B1} can be hydrogen. When B² is any of the aforementioned moieties shown above, in some embodiments, D² can be oxygen.

An embodiment disclosed herein relates to a compound of Formula (III), or a pharmaceutically acceptable salt, prodrug or prodrug ester thereof:

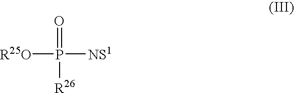

wherein: NS¹ can be a nucleoside attached to the phosphorus via the oxygen bonded to the 5′-carbon; R²⁵ can be

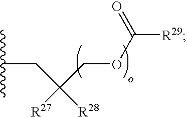

R²⁶ can be an —N-linked amino acid; R²⁷ and R²⁸ can be each independently —C≡N or an optionally substituted substituent selected from C₁₋₈ organylcarbonyl, C₁₋₈ alkoxycarbonyl and C₁₋₈ organylaminocarbonyl; R²⁹ can be hydrogen or an optionally substituted C₁₋₄-alkyl; and o can be 1 or 2.

Example of R²⁵ groups, include but are not limited to the following:

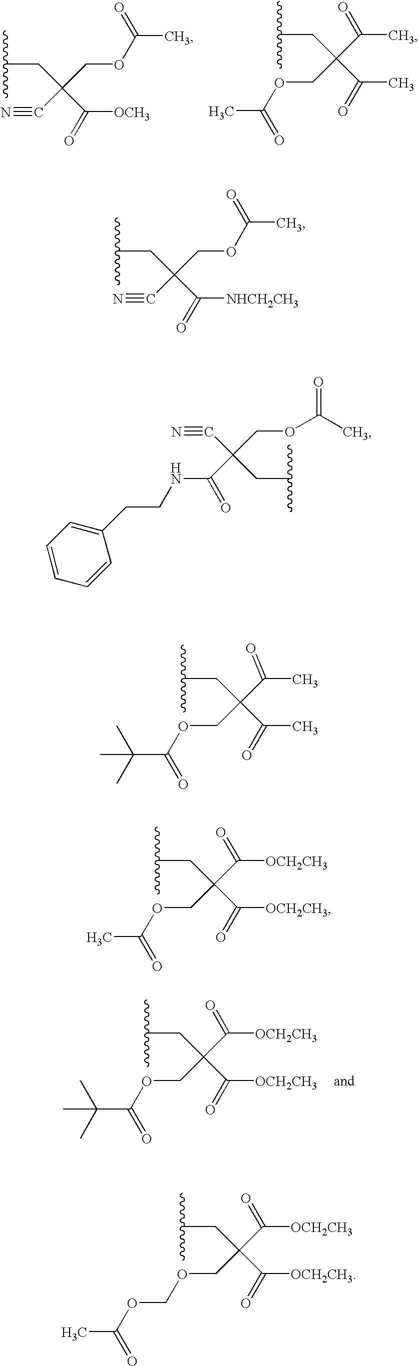

In an embodiment, R²⁵ can be

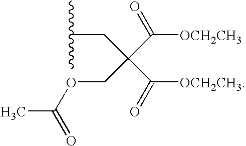

In another embodiment, R²⁵ can be

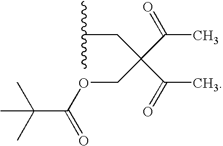

In still another embodiment, R²⁵ can be

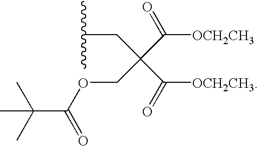

In yet still another embodiment, R²⁵ can be

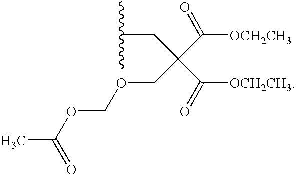

In some embodiments, R²⁵ can be

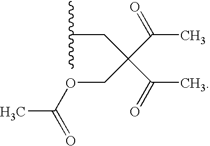

Various amino acids can be used for the substituent indicated by R²⁶ In some embodiments, R²⁶ can have the structure

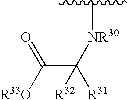

wherein: R³⁰ can be hydrogen or an optionally substituted C₁₋₄-alkyl; R³¹ can be selected from hydrogen, an optionally substituted C₁₋₆-alkyl, an optionally substituted aryl, an optionally substituted aryl(C₁₋₄ alkyl) and haloalkyl; R³² can be hydrogen or an optionally substituted C₁₋₄-alkyl; and R³³ can be selected from an optionally substituted C₁₋₆ alkyl, an optionally substituted C₆ aryl, an optionally substituted C₁₀ aryl, and an optionally substituted C₃₋₆ cycloalkyl. In an embodiment, R³⁰ can be hydrogen. In some embodiments, R³¹ can be an optionally substituted C₁₋₄-alkyl, for example, methyl. In an embodiment, R³² can be hydrogen or an optionally substituted C₁₋₄-alkyl. In some embodiment, R³³ can be an optionally substituted C₁₋₄-alkyl such as methyl. One example of a suitable R²⁶ group includes, but are not limited to,

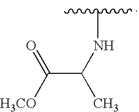

In an embodiment, the amino acid can be in the L-configuration. In another embodiment, the amino acid can be in the D-configuration. For example, R²⁶ can be

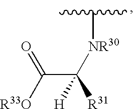

such as

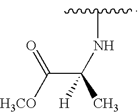

In some embodiments, NS¹ can be selected from adenosine, guanosine, 5-methyluridine, uridine, cytidine and derivatives thereof. In an embodiment, NS¹ can have the structure of Formula (IV). Additional suitable amino acids are described herein.

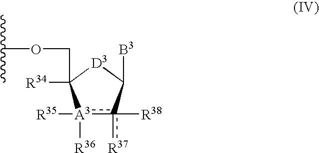

wherein: each [symbol] can be a double or single bond; A³ can be selected from C (carbon), O (oxygen) and S (sulfur); B³ can be an optionally substituted heterocyclic base or an optionally substituted heterocyclic base derivative thereof; D³ can be selected from C═CH₂, CH₂, O (oxygen) and S (sulfur); R³⁴ selected from hydrogen, azido, —CN, an optionally substituted C₁₋₄ alkyl and an optionally substituted C₁₋₄ alkoxy; R³⁵ can be absent or selected from hydrogen, halogen, hydroxy and an optionally substituted C₁₋₄ alkyl; R³⁶ can be absent or selected from hydrogen, halogen, azido, amino, hydroxy and an —O-linked amino acid; R³⁷ can be selected from hydrogen, halogen, hydroxy, —CN, —NC, an optionally substituted C₁₋₄ alkyl, an optionally substituted C₁₋₄ alkoxy and an —O-linked amino acid; and R³⁸ can be absent or selected from hydrogen, halogen, hydroxy, —CN, —NC, an optionally substituted C₁₋₄ alkyl, an optionally substituted haloalkyl and an optionally substituted hydroxyalkyl, or when the bond to R³⁷ indicated by [symbol] is a double bond, then R³⁷ is a C₁₋₄ alkenyl and R³⁸ is absent.

In an embodiment, o can be 1. In another embodiment, o can be 2. In some embodiments, A³ can be carbon. In some embodiments, each [symbol] can be a single bond. In an embodiment, A³ can be carbon and D³ can be oxygen. In other embodiments, A³ can be carbon, D³ can be oxygen and o can be 1. In an embodiment, A³ can be carbon, D³ can be oxygen and o can be 2. In an embodiment, A³ can be carbon, D³ can be oxygen, o can be 1 and each [symbol] can be a single bond. In another embodiment, A³ can be carbon, D³ can be oxygen, o can be 2 and each [symbol] can be a single bond.

The substituent B³ can also vary. In some embodiments, B³ can be selected from:

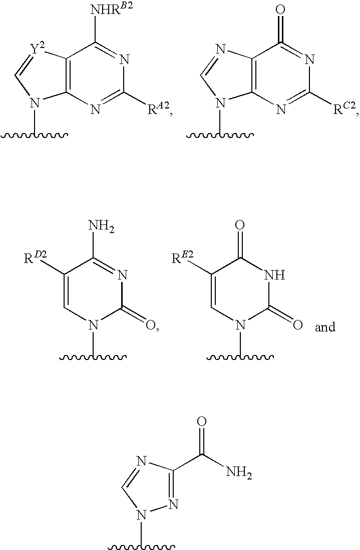

wherein: R^{A2} can be hydrogen or halogen; R^{B2} can be hydrogen, an optionally substituted C₁₋₄alkyl, or an optionally substituted C₃₋₈ cycloalkyl; R^{C2} can be hydrogen or amino; R^{D2} can be hydrogen or halogen; R^{E2} can be hydrogen or an optionally substituted C₁₋₄alkyl; and Y² can be N (nitrogen) or CR^{F2}, wherein R^{F2} can be selected from hydrogen, halogen, an optionally substituted C₁₋₄-alkyl, an optionally substituted C₂₋₄-alkenyl and an optionally substituted C₂₋₄-alkynyl. In some embodiments, B³ can be

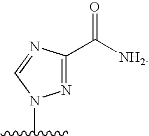

In other embodiments, B³ can be

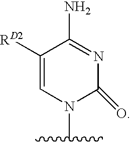

In yet other embodiments, B³ can be

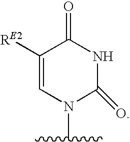

In yet still other embodiments, B³ can be

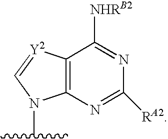

In an embodiment Y² can be nitrogen; R^{A2} can be hydrogen and R^{B2} can be hydrogen. In another embodiment, Y² can be CR^{F2}, wherein R^{F2} can be selected from hydrogen, halogen, an optionally substituted C₁₋₄-alkyl, an optionally substituted C₂₋₄-alkenyl and an optionally substituted C₂₋₄-alkynyl; R^{A2} can be hydrogen and R^{B2} can be hydrogen. When B³ is any of the aforementioned moieties shown above, in some embodiments, A³ can be carbon. In an embodiment, B³ can be any of the aforementioned moieties shown above, A³ can be carbon and D³ can be oxygen. In some embodiments, B³ can be any of the aforementioned moieties shown above, A³ can be carbon, D³ can be oxygen and each [symbol] can be a single bond.

In an embodiment, R³⁶ can be hydroxy. In another embodiment, R³⁶ can be an —O-linked amino acid. In some embodiments, R³⁷ can be hydroxy. In other embodiments, R³⁷ can be a C₁₋₄ alkoxy such as methoxy. In still other embodiments, R³⁷ can be an —O-linked amino acid. A non-limiting list of suitable —O-linked amino acid include, but are not limited to the following: alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. In an embodiment, the —O-linked amino acid can be valine. In some embodiments, the —O-linked amino acid can be selected from —O-linked α-amino acid, —O-linked β-amino acid, —O-linked γ-amino acid and —O-linked δ-amino acid. In an embodiment, the —O-linked amino acid can be in the L-configuration. In some embodiments, both R³⁶ and R³⁷ can be hydroxy groups. In other embodiments, R³⁶ can be a hydroxyl group and R³⁷ can be —O-linked amino acid.

In another embodiment, NS¹ can have the structure of Formula (V).

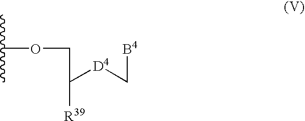

wherein: B⁴ can be an optionally substituted heterocyclic base or an optionally substituted heterocyclic base derivative thereof; D⁴ can be selected from C═CH₂, CH₂, O (oxygen) and S (sulfur); and R³⁹ can be hydrogen or —(CH₂)—OH. In an embodiment, D⁴ can be oxygen.

As with B³, the substituent B⁴ can also vary. In some embodiments, B⁴ can be selected from:

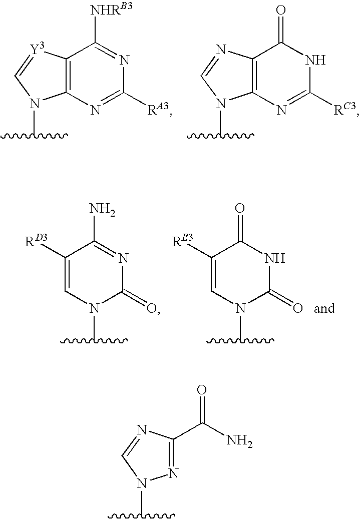

wherein: R^{A3} can be hydrogen or halogen; R^{B3} can be hydrogen, an optionally substituted C₁₋₄alkyl, or an optionally substituted C₃₋₈ cycloalkyl; R^{C3} can be hydrogen or amino; R^{D3} can be hydrogen or halogen; R^{E3} can be hydrogen or an optionally substituted C₁₋₄alkyl; and Y³ can be N (nitrogen) or CR^{F3}, wherein R^{F3} can be selected from hydrogen, halogen, an optionally substituted C₁₋₄-alkyl, an optionally substituted C₂₋₄-alkenyl and an optionally substituted C₂₋₄-alkynyl. In some embodiments, B⁴ can be

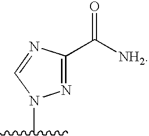

In other embodiments, B⁴ can be

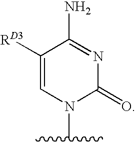

In yet other embodiments, B⁴ can be

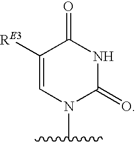

In yet still other embodiments, B⁴ can be

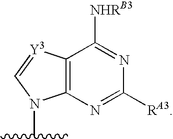

In an embodiment Y³ can be nitrogen; R^{A3} can be hydrogen and R^{B3} can be hydrogen. In another embodiment, Y³ can be CR^{F3}, wherein R^{F3} can be selected from hydrogen, halogen, an optionally substituted C₁₋₄-alkyl, an optionally substituted C₂₋₄-alkenyl and an optionally substituted C₂₋₄-alkynyl; R^{A3} can be hydrogen and R^{B3} can be hydrogen. When B⁴ is any of the aforementioned moieties shown above, in some embodiments, D⁴ can be oxygen.

Examples of compounds of Formulae (I), (II) and (III) are shown below. The compounds shown below are examples and do not represent all compounds of Formulae (I), (II) and (III).

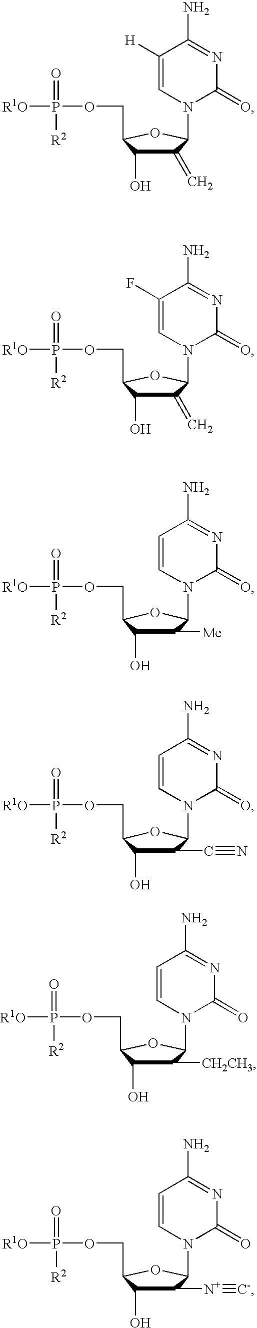

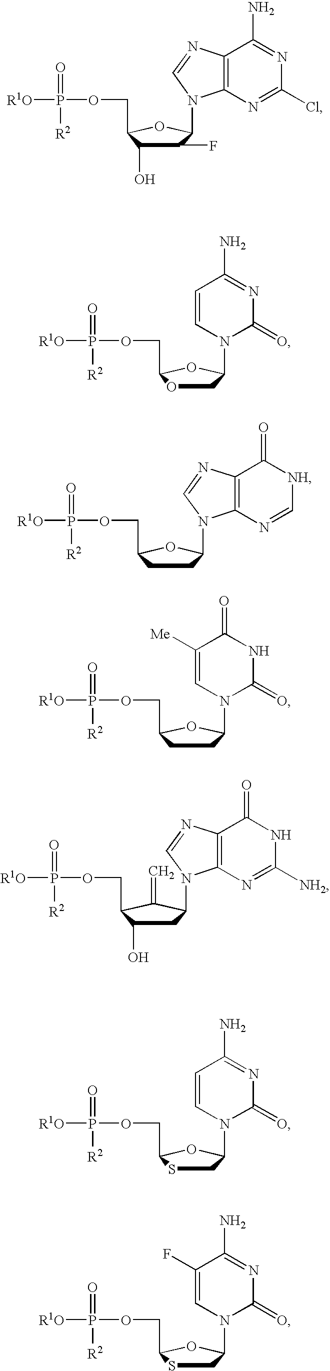

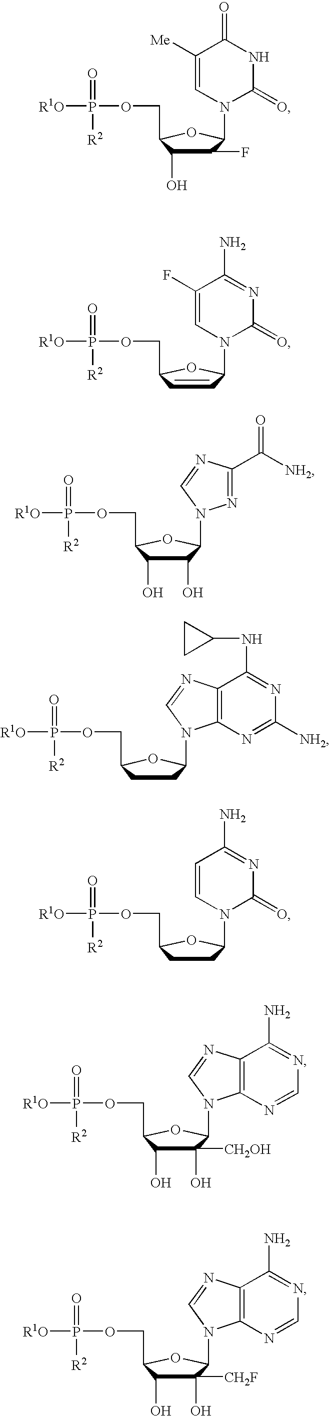

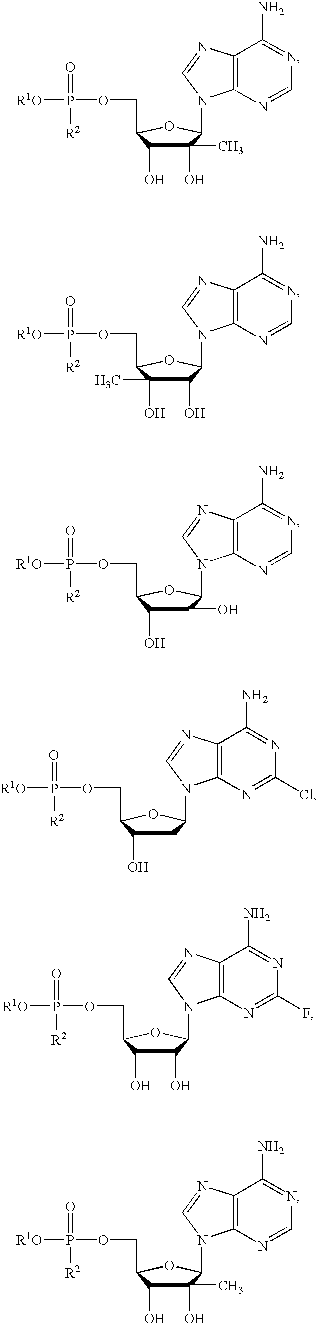

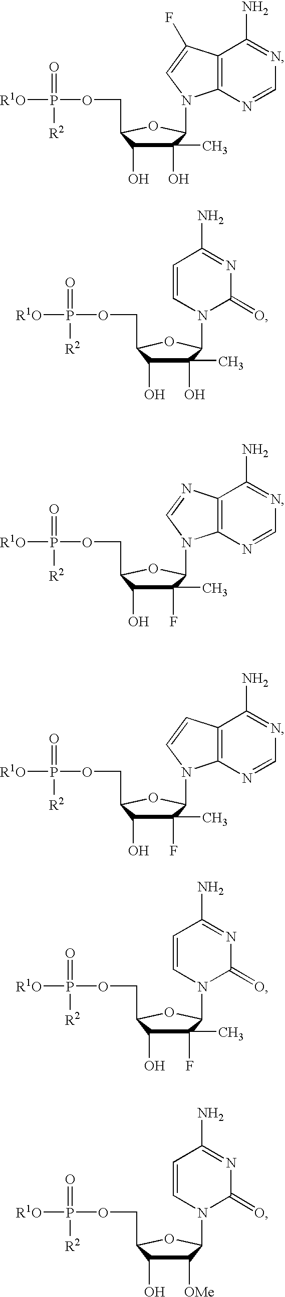

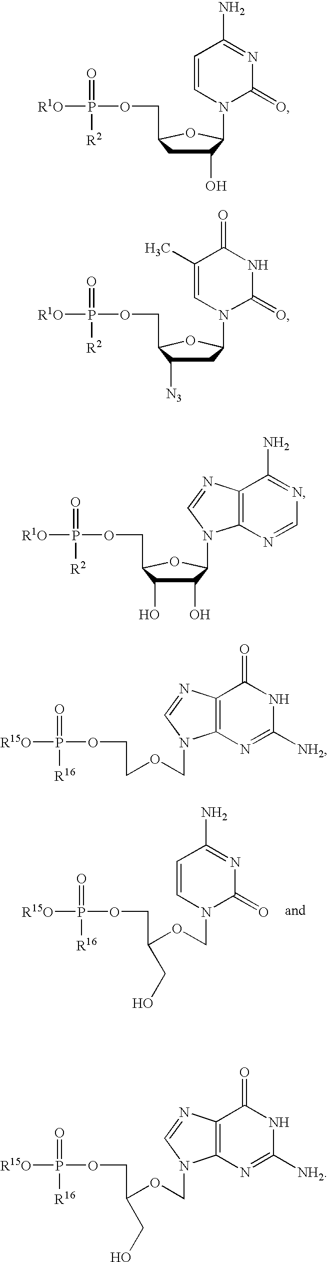

In an embodiment, compounds of Formulae (I) and (III) can be one of the following compounds:

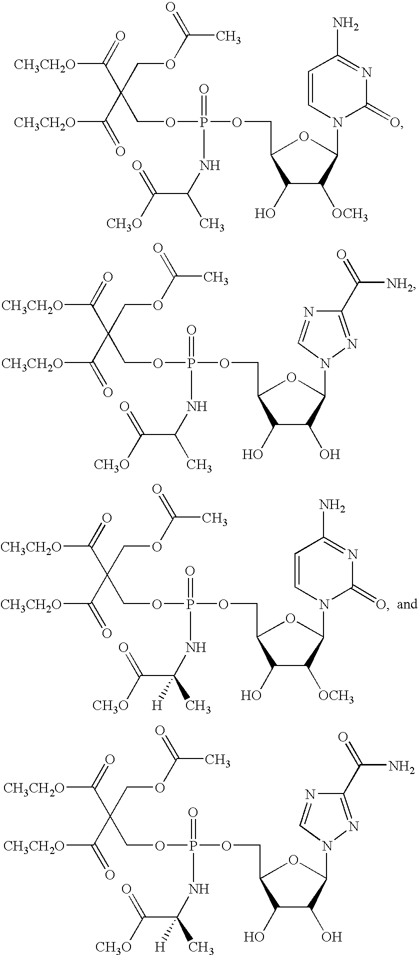

In some embodiments, the 2,2-disubstituted-acyl(oxyalkyl) groups disclosed herein, such as

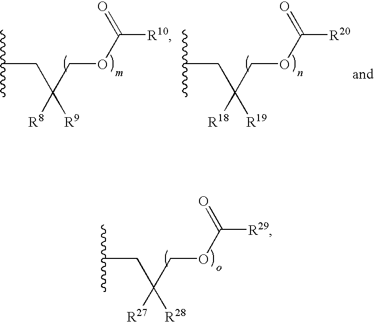

and an amino acid, such as

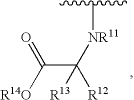

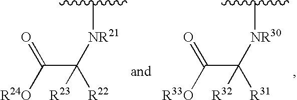

can be linked to a polynucleotide, an oligonucleotide, or an analog thereof.

As used herein, the term “polynucleotide” refer to a polymeric compound made up of any number of covalently bonded nucleotide monomers. Examples of polynucleotides include, but are not limited to, DNA, RNA, oligonucleotides, hybrids of RNA, hybrids of DNA, ribozymes, antisense molecules (e.g., siRNA, miRNA, shRNA, piRNA, and the like), decoy nucleic acids, and the like.

With respect to DNA and RNA, in an embodiment, the DNA or RNA can be single stranded. In another embodiment, the DNA or RNA can be double-stranded. In still another embodiment, the DNA or RNA can be triple-stranded. As used herein, a double-stranded polynucleotide comprises a first single-stranded polynucleotide and a second single-stranded polynucleotide in which at least a portion of the first single-stranded polynucleotide is capable of hybridizing with at least a portion of the second single-stranded polynucleotide. It is not necessary that the first and the second single-stranded polynucleotides in a double-stranded polynucleotide or duplex are 100% complementary. The first single-stranded polynucleotide has to be complementary to a certain degree with at least a portion of the second single-stranded polynucleotide. The percentage of (overall) complementarity of two strands of polynucleotides is preferably at least 50%, preferably at least 70%, or more preferably at least 90%. The term “double stranded” also includes polynucleotide hairpin constructs, such as short-hairpins. The term “double stranded” also includes duplex polynucleotide (or short-hairpins) with an overhang. In other words, the double stranded polynucleotides or duplexes not need to be 100% double stranded in the strict sense.

Oligonucleotides are typically made up of a relatively small number of nucleotide monomers. In some embodiments, the oligonucleotide has no more than 30 nucleic acid molecules. In other embodiments, the oligonucleotide has 5-10 nucleic acid molecules. In other embodiments, the oligonucleotide has 10-20 nucleic acid molecules. In still other embodiments, the oligonucleotide has 20-30 nucleic acid molecules.

In some embodiments, the polynucleotides and oligonucleotides can be linear. In other embodiments, the polynucleotides and oligonucleotides may be circular. The polynucleotides and oligonucleotides described herein can include DNA, RNA or a hybrid thereof, where the nucleic acid may contain combination of deoxyribo- and ribo-nucleotides, and combination of bases including uracil, adenine, thymine, cytosine, guanine, inosine, xanthine, thypoxanthine, isocysteine, isoguaninne, and the like. The polynucleotides and oligonucleotides can include mixtures of naturally occurring nucleotides and modified nucleotides having non-naturally-occurring portions which function similarly. Alternatively, mixtures of different modified nucleotides, and mixtures of naturally occurring nucleotides can be used. Modified or substituted polynucleotide can be advantageous over native forms because of desirable properties such as, for example, enhanced cellular uptake, enhanced binding ability to target, improved pharmacokinetics, and increased stability in the presence of nucleases.

As used herein, the term “ribozyme” is an abbreviation for “ribonucleic acid enzyme,” also sometimes known as “RNA enzyme” or “catalytic RNA,” and refer to a class of RNA molecules capable of catalyzing a chemical reaction. Many natural ribozymes catalyze either the hydrolysis of one of their own phosphodiester bonds, or the hydrolysis of bonds in other RNAs. They have also been found to catalyze the aminotransferase activity of the ribosome. Some ribozymes may play an important role as therapeutic agents, as enzymes which tailor defined RNA sequences, as biosensors, and for applications in functional genomics and gene discovery.

As used herein, the term “siRNA” is an abbreviation for “short interfering RNA,” also sometimes known as “small interfering RNA” or “silencing RNA,” and refers to a class of about 19-25 nucleotide-long double-stranded RNA molecules in eukaryotes that are involved in the RNA interference (RNAi) pathway that results in post-transcriptional sequence-specific gene silencing. After being processed by the RNAase III enzyme Dicer, siRNAs can hybridize to cognate mRNAs having a sequence homologous to the siRNA sequence and induce mRNA cleavage and degradation.

As used herein, the term “miRNA” is an abbreviation for “microRNA,” and refers to a class of about 21-25 nucleotide-long single-stranded RNA molecules, which plays a role in regulating gene expression. miRNAs are non-coding RNAs that are encoded by genes from whose DNA they are transcribed. Instead of being translated into protein, each primary transcript (a pri-miRNA), which may have a length of greater than 100 nucleotides, is processed into a short stem-loop structure called a pre-miRNA. Pre-miRNAs usually have a length of 50-90 nucleotides, particularly 60-80 nucleotides, and are processed into functional miRNAs. Mature miRNAs are capable of causing post-transcriptional silencing of target genes which have complete or partially complementary sequences to the miRNAs. Preferably, the regions of complementarity are at least 8 to 10 nucleotides long.

As used herein, the term “shRNA” is an abbreviation for “small hairpin RNA,” also sometimes known as “short hairpin RNA.” shRNA is a sequence of RNA that contains a sense sequence, an antisense sequence, and a short loop sequence between the sense and antisense sequences. Because of the complementarity of the sense and antisense sequences, shRNA molecules tend to form hairpin-shaped double-stranded RNA (dsRNA). shRNAs are processed by the RNAase III enzyme Dicer into siRNA which then get incorporated into the RNA-induced silencing complex (RISC) to silence gene expression via RNA interference.

As used herein, the term “piRNA” is an abbreviation for “Piwi-interacting RNA (piRNA),” and refers to a class of small RNA molecules that is expressed in mammalian testes and somatic cells and forms RNA-protein complexes with Piwi proteins. The Piwi proteins are part of a family of proteins called the argonautes, which are active in the testes of mammals and are required for germ-cell and stem-cell development in invertebrates. piRNA has a role in RNA silencing of retrotransposons and other genetic elements in germ line cells via the formation of an RNA-induced silencing complex (RISC). piRNAs are short stretches of RNAs with a typical length of 25-33 nucleotides, making them distinct entities from miRNAs and siRNAs.

As used herein, the term “decoy nucleic acids” refers to a class of nucleic acids that resembles a natural nucleic acid, but is modified to inhibit or interrupt the activity of the natural nucleic acid. A non-limiting list of decoy nucleic acids includes decoy RNA and decoy DNA. For instance, a decoy RNA can mimic the natural binding domain for a ligand, compete with the natural binding target for the binding of a specific ligand, and thereby prevent the natural binding target from binding the specific ligand. A decoy DNA which contains the specific sequence recognized by a transcription factor can compete with the natural binding target sequence for the binding of the transcription factor and thus block transcription.

In some embodiments, one or more of the aforementioned 2,2-disubstituted-acyl(oxyalkyl) groups and one or more amino acids described herein can be present in polynucleotides that have a length of about 5 to about 10 nucleotides, about 10 to about 15 nucleotides, about 15 to about 20 nucleotides, about 20 to about 25 nucleotides, about 25 to about 30 nucleotides, about 30 to about 35 nucleotides, about 35 to about 40 nucleotides, about 40 to about 45 nucleotides, about 45 to about 50 nucleotides, about 55 to about 60 nucleotides, about 60 to about 65 nucleotides, about 65 to about 70 nucleotides, about 70 to about 75 nucleotides, about 75 to about 80 nucleotides, about 80 to about 85 nucleotides, about 85 to about 90 nucleotides, about 90 to about 95 nucleotides, about 95 to about 100 nucleotides, about 105 to about 110 nucleotides, about 110 to about 130 nucleotides, about 130 to about 150 nucleotides, about 150 to about 170 nucleotides, about 170 to about 190 nucleotides, about 190 to about 210 nucleotides, or longer, or any number in between, including full length genes or RNA transcripts thereof. In some embodiments, the polynucleotide can have, for example, a length of about 18 to about 100 nucleotides, preferably from about 18 to about 80 nucleotides, more preferably from about 18 to about 90 nucleotides, most preferably from about 19 to about 25 nucleotides, particularly 19, 20, 21, 22, 23, 24, or 25 nucleotides.

The 2,2-disubstituted-acyl(oxyalkyl) groups and the amino acids disclosed herein can be present within only one strand or in both strands of the polynucleotide. In some embodiments, only one 2,2-disubstituted-acyl(oxyalkyl) group disclosed herein can be present within a polynucleotide. In other embodiments, a plurality of 2,2-disubstituted-acyl(oxyalkyl) groups disclosed herein can be present within a polynucleotide. In an embodiment, only one amino acid disclosed herein can be present within a polynucleotide. In other embodiments, a plurality of amino acids disclosed herein can be present within a polynucleotide. In some embodiments, a 2,2-disubstituted-acyl(oxyalkyl) group disclosed herein can be present on every other phosphate group of a polynucleotide. In some embodiments, a plurality of 2,2-disubstituted-acyl(oxyalkyl) groups disclosed herein can be present on about 5 to about 95% of the phosphate groups of a polynucleotide, more preferably about 10 to about 70% of the phosphate groups of a polynucleotide, yet more preferably about 15 to about 50% of the phosphate groups of a polynucleotide, most preferably about 20 to about 40% of the phosphate groups of a polynucleotide. In some embodiments, an amino acid disclosed herein can be present on every other phosphate group of a polynucleotide. In some embodiments, a plurality of an amino acids disclosed herein can be present on about 5 to about 95% of the phosphate groups of a polynucleotide, more preferably about 10 to about 70% of the phosphate groups of a polynucleotide, yet more preferably about 15 to about 50% of the phosphate groups of a polynucleotide, most preferably about 20 to about 40% of the phosphate groups of a polynucleotide.

The skilled artisan will appreciate the polynucleotides (e.g., oligonucleotides, dsDNA, ssDNA, dsRNA, ribozyme, siRNA, miRNA, shRNA, piRNA, decoy nucleic acids, and the like) are not limited by any particular sequence. In some embodiments, the polynucleotides can comprise coding sequence. In some embodiments, the polynucleotides can comprise noncoding sequence, such as regulatory sequence, including a promoter sequence or a promoter-enhancer combination. The polynucleotides can be of any length and can be used in different application such as gene therapy, modulation of gene expression, and gene detection. Polynucleotides also can be useful for diagnostics, therapeutics, prophylaxis, and research can be used in the methods and compounds disclosed herein.

Non-limiting examples of ribozyme, siRNA, shRNA, miRNA, and piRNA molecules useful in the embodiments described herein include those disclosed in databases such as Riboapt DB (http://mcbc.usm.edu/riboaptDB/), siRecords (http://siRecords.umn.edu/siRecords), siRNAdb (http://sirna.sbc.su.se/), RNAi Codex database (http://codex.cshl.edu/scripts/newmain.pl), shRNA Clone Library (http://cgap.nci.nih.gov/RNAi/RNAi2), miRBase (http://microrna.sanger.ac.uk/), piRNABank (http://pirnabank.ibab.ac.in/), and RNAdb (http://research.imb.uq.edu.au/rnadb/). Although examples of noncoding RNA including ribozyme, siRNA, shRNA, miRNA, and piRNA molecules are described herein, the skilled artisan will readily appreciate that the compounds and methods disclosed herein are useful for any polynucleotides such as ribozymes, siRNAs, miRNAs, shRNAs, piRNA, dsRNAs, RNAi's, and oligonucleotides now known or discovered in the future. In a general sense, the operability of the methods and compounds disclosed herein is not dependent on the sequence or function of the polynucleotides. Rather, the disclosed methods and compounds are useful for delivering polynucleotides into a cell.

Neutralizing the charge on the phosphate group may facilitate the penetration of the cell membrane by compounds of Formulae (I), (II) and (III) by making the compound more lipophilic. Furthermore, it is believed that the 2,2-disubstituted-acyl(oxyalkyl) groups, such as

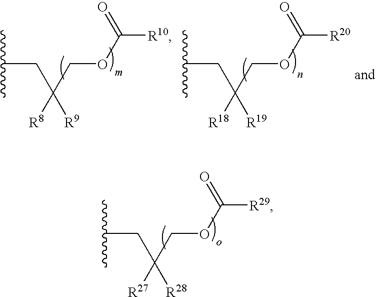

attached to the phosphate impart increased plasma stability to compounds of Formulae (I), (II) and (III) by inhibiting the degradation of the compound. Once inside the cell, the 2,2-disubstituted-acyl(oxyalkyl) group attached to the phosphate can be easily removed by esterases via enzymatic hydrolysis of the acyl group. The remaining portions of the group on the phosphate can then be removed by elimination. The general reaction scheme is shown below in Scheme 1a.

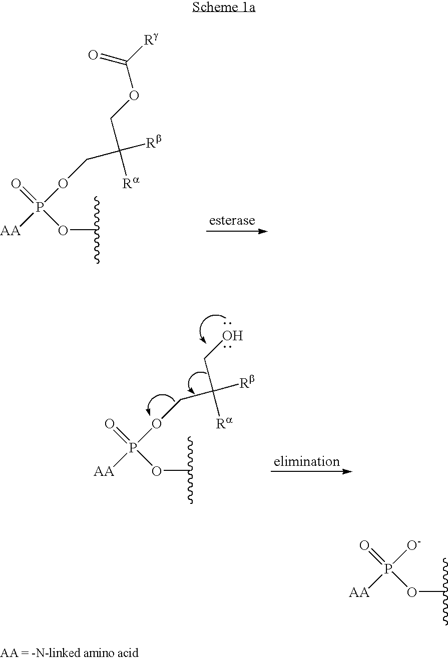

A further advantage of the 2,2-disubstituted-acyl(oxyalkyl) groups described herein is the rate of elimination of the remaining portion of the 2,2-disubstituted-acyl(oxyalkyl) group is modifiable. Depending upon the identity of the substituents on the 2-carbon, shown in Scheme 1a as R^{α} and R^{β}, the rate of elimination may be adjusted from several seconds to several hours. As a result, the removal of the remaining portion of the 2,2-disubstituted-acyl(oxyalkyl) group can be retarded, if necessary, to enhance cellular uptake but, readily eliminated upon entry into the cell.

Phosphoamidates of nucleosides have been shown to have increased efficacy compared to their parent nucleosides. After penetration into the cell, esterases can initiate the cleavage of the amino acid as shown in Scheme 1b below. As with a 2,2-disubstituted-acyl(oxyalkyl) group, the cleavage rate of the amino acid can be increased or decreased depending upon the substituents present on the amino acid. Accordingly, the cleavage of the amino acid can be modified. By changing the cleavage rate of the amino acid, release of the phosphorylated nucleoside can be varied.

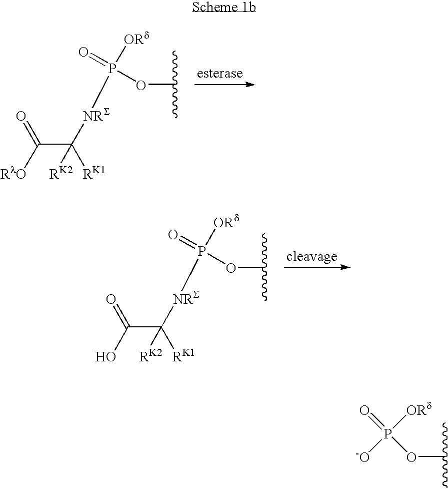

Upon removal of the 2,2-disubstituted-acyl(oxyalkyl) group and the amino acid, the resulting nucleotide analog possesses a monophosphate. Thus, the necessity of an initial intracellular phosphorylation is no longer a prerequisite to obtaining the biologically active phosphorylated form. In some embodiments, the 2,2-disubstituted-acyl(oxyalkyl) group can be removed before the amino acid. In other embodiments, the 2,2-disubstituted-acyl(oxyalkyl) group can be removed after the amino acid. In still other embodiments, the 2,2-disubstituted-acyl(oxyalkyl) group can be removed at approximately the same time.

### Synthesis

Compounds of Formulae (I), (II) and (III), and those described herein may be prepared in various ways. General synthetic routes to the compounds of Formulae (I), (II) and (III), and the starting materials used to synthesize the compounds of Formulae (I), (II) and (III) are shown in Schemes 2a, 2b and 2c. The routes shown are illustrative only and are not intended, nor are they to be construed, to limit the scope of the claims in any manner whatsoever. Those skilled in the art will be able to recognize modifications of the disclosed synthesis and to devise alternate routes based on the disclosures herein; all such modifications and alternate routes are within the scope of the claims.

The hydroxy precursors,

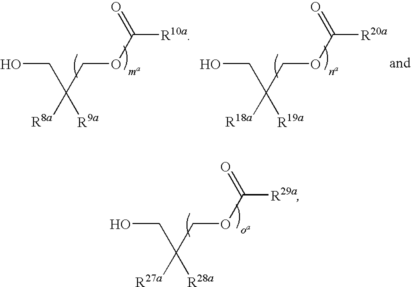

in which R^{8a}, R^{9a}, R^{10a}, R^{18a}, R^{19a}, R^{20a}, R^{27a}, R^{28a}, R^{29a}, m^{b}, n^{a} and o^{a} are the same as R⁸, R⁹, R¹⁰, R¹⁸, R¹⁹, R²⁰, R²⁷, R²⁸, R²⁹, m, n and o, respectively, as described herein, of the 2,2-disubstituted-acyl(oxyalkyl) groups can be synthesized according in a manner similar to those described in the following articles. Ora, et al., J. Chem. Soc. Perkin Trans. 2, 2001, 6, 881-5; Poijärvi, P. et al., Helv. Chim. Acta. 2002, 85, 1859-76; Poijärvi, P. et al., Lett. Org. Chem., 2004, 1, 183-88; and Poijärvi, P. et al., Bioconjugate Chem., 2005 16(6), 1564-71, all of which are hereby incorporated by reference in their entireties.

Examples of hydroxy precursors can include the following:

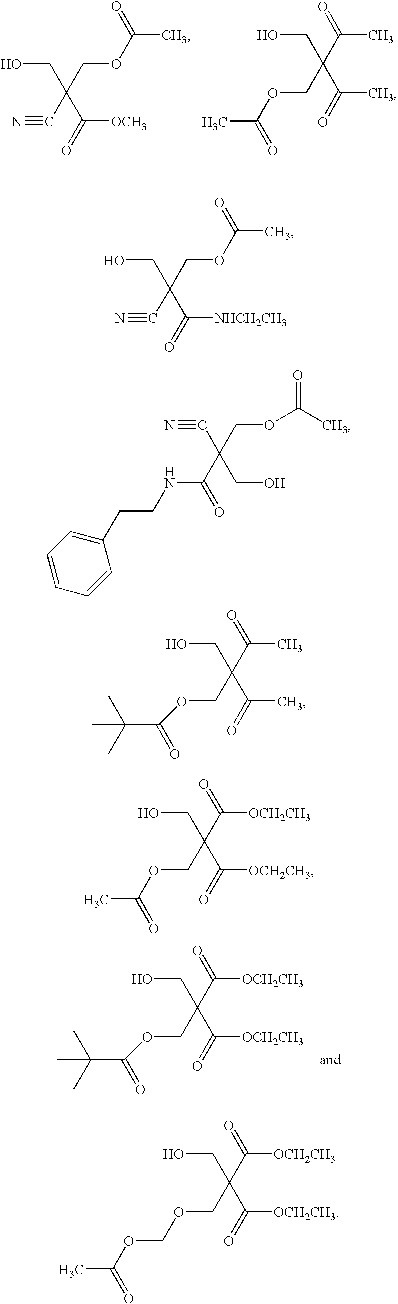

In an embodiment, the hydroxy precursor can be

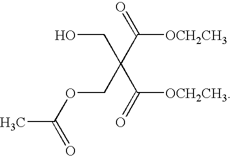

In another embodiment, the hydroxy precursor can be

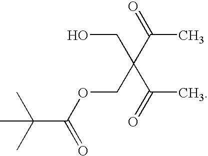

In still another embodiment, the hydroxy precursor can be

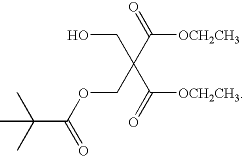

In an embodiment, the hydroxy precursor can be

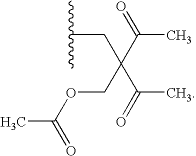

In yet still another embodiment, the hydroxy precursor can be

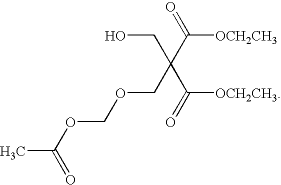

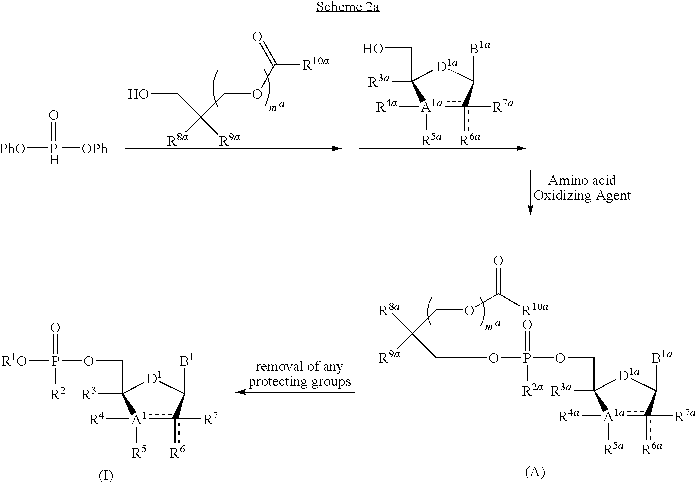

One embodiment disclosed herein relates to a method of synthesizing a compound of Formula (I) that can include the transformations shown in Scheme 2a. In Scheme 2a, A^{1a}, D^{1a}, R^{2a}, R^{3a}, R^{4a}, R^{7a}, R^{8a}, R^{9a}, R^{10a} and m^{a} can be the same as A¹, D¹, R², R³, R⁴, R⁷, R⁸, R⁹, R¹⁰ and m, respectively, as described above with respect to Formula (I). In Scheme 2a, in some embodiments, R^{5a} can be absent or selected from hydrogen, halogen, azido, amino, hydroxy, an —O-linked amino acid and O-PG¹, wherein if R^{5a} is not a hydroxy group, then R^{5a} can be R⁵. Additionally, in Scheme 2a, R^{6a} can be selected from absent or selected from hydrogen, halogen, hydroxy, —CN, —NC, an optionally substituted C₁₋₄ alkyl, an optionally substituted C₁₋₄ alkoxy, an —O-linked amino acid and O-PG², wherein if R^{6a} is not a hydroxy group, then R^{6a} can be R⁶. In some embodiments, B^{1a} can be an optionally substituted heterocyclic base, an optionally substituted heterocyclic base derivative, an optionally substituted protected heterocyclic base, or an optionally substituted protected heterocyclic base derivative, and if B^{1a} does not have one or more amino groups attached to a ring protected with one or more protecting groups and/or any —NH groups present in a ring of B^{1a} protected with one or more protecting groups, then B^{1a} can be B¹. If more than one protecting group is present on B^{1a}, the protecting groups can be the same or different.

Various protecting groups can be used to protect the oxygen of any hydroxy groups attached to the 2′- and 3′-carbons. In some embodiments, PG¹ can be a triarylmethyl or levulinoyl protecting group. In some embodiments, PG² can be a triarylmethyl or levulinoyl protecting group. In an embodiment, PG¹ and PG² can be levulinoyl protecting groups. By protecting the oxygens on the 2′- and 3′-carbons, various undesirable side-reactions can be prevented or reduced. By reducing and/or elimination the formation of unwanted side products, the separation of the desired compound can be more facile.

Similarly, various protecting groups can be used to protect the optionally substituted heterocyclic base and/or optionally substituted heterocyclic base derivative. For example, one or more amino groups attached to a ring and/or any —NH groups present in a ring of the optionally substituted heterocyclic base and/or optionally substituted heterocyclic base derivative can be protected with one or more suitable protecting groups. In an embodiment, the optionally substituted heterocyclic base and/or optionally substituted heterocyclic base derivative can be protected with one or more triarylmethyl protecting groups. A non-limiting list of triarylmethyl protecting groups are trityl, monomethoxytrityl (MMTr), 4,4′-dimethoxytrityl (DMTr), 4,4′,4″-trimethoxytrityl (TMTr), 4,4′,4″-tris-(benzoyloxy) trityl (TBTr), 4,4′,4″-tris (4,5-dichlorophthalimido) trityl (CPTr), 4,4′,4″-tris (levulinyloxy) trityl (TLTr), p-anisyl-1-naphthylphenylmethyl, di-o-anisyl-1-naphthylmethyl, p-tolyldipheylmethyl, 3-(imidazolylmethyl)-4,4′-dimethoxytrityl, 9-phenylxanthen-9-yl (Pixyl), 9-(p-methoxyphenyl) xanthen-9-yl (Mox), 4-decyloxytrityl, 4-hexadecyloxytrityl, 4,4′-dioctadecyltrityl, 9-(4-octadecyloxyphenyl) xanthen-9-yl, 1,1′-bis-(4-methoxyphenyl)-1′-pyrenylmethyl, 4,4′,4″-tris-(tert-butylphenyl)methyl (TTTr) and 4,4′-di-3,5-hexadienoxytrityl.

As shown in Scheme 2a, diphenylphosphite can be reacted with

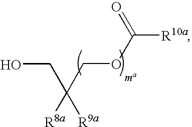

a nucleoside of having the structure

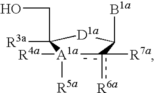

an amino acid, and a suitable oxidizing agent to form a compound of Formula (A).

As previously discussed, various amino acids can be used. In some embodiments, the starting amino acid can have the following structure

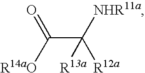

wherein R^{11a}, R^{12a}, R^{13a} and R^{13a} can be the same as R¹¹, R¹², R¹³ and R¹⁴, as described herein with respect to Formula (I).

Any suitable oxidizing agent can be used. In an embodiment, the oxidizing agent can be carbon tetrachloride (CCl₄). In some embodiments, the oxidizing agent, such as CCl₄, oxidizes the phosphorus from (III) to (V).

If the substituent attached to the 3′-carbon is a protected oxygen on the compound of Formula (A), the protecting group, PG¹, can be removed, and if the substituent attached to the 2′-position is a protected oxygen on the compound of Formula (A), the protecting group, PG², can be removed to form the compound of Formula (I) as described herein. In an embodiment, when PG¹ and PG² are the levulinoyl group(s), the levulinoyl groups can be removed with hydrazinium acetate. Likewise, if there are any protecting groups present on the optionally substituted heterocyclic base or optionally substituted heterocyclic base derivative, the protecting group(s) can be removed with one or more suitable reagents. For example, when the protecting group(s) is/are triarylmethyl protecting group(s), the protecting group(s) can be removed with an acid. In an embodiment, the acid can be acetic acid. In some embodiments, the protecting groups can be removed sequentially. In other embodiments, the protecting groups can be removed simultaneously. In an embodiment, the protecting group(s), if present, attached to the 2′- and/or 3′-carbons can be removed before any protecting groups present on the optionally substituted heterocyclic base or optionally substituted heterocyclic base derivative. In another embodiment, the protecting group(s), if present, attached to the 2′- and/or 3′-carbons can be removed after any protecting groups present on the optionally substituted heterocyclic base or optionally substituted heterocyclic base derivative. In still another embodiment, the protecting group(s), if present, attached to the 2′- and/or 3′-carbons can be removed at approximately the same time as any protecting groups present on the optionally substituted heterocyclic base or optionally substituted heterocyclic base derivative. In some embodiments, when both the oxygens attached to the 2′- and/or 3′-carbons are protected, the protecting groups can be removed at approximately the same time. For example, when the protecting groups attached to oxygens 2′- and 3′-positions are levulinoyl groups, both groups can be removed approximately at the same time with hydrazinium acetate. If the substituents attached to the 2′- and 3′-positions are not protected oxygens and one or more amino groups attached to a ring and/or any —NH groups present in a ring of the optionally substituted heterocyclic base or optionally substituted heterocyclic base derivative are not protected, the compound of Formula (A) can be a compound of Formula (I).

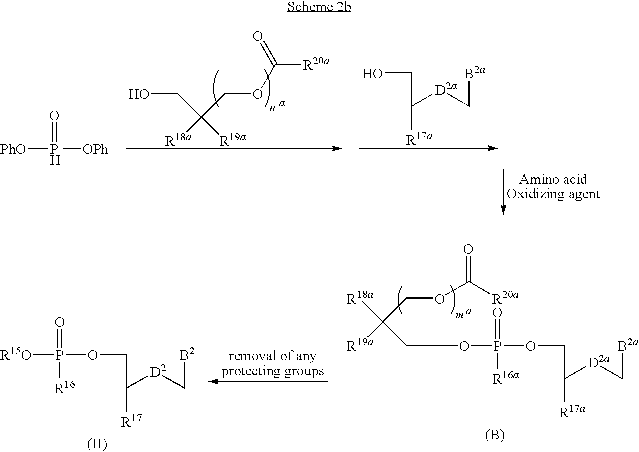

Some embodiments disclosed herein relate to a method of synthesizing a compound of Formula (II) that can include the transformations shown in Scheme 2b. In Scheme 2b, D^{2a}, R^{16a}, R^{18a}, R^{19a}, R^{20a} and n^{a} can be the same as D², R¹⁶, R¹⁸, R¹⁹, R²⁰ and n, respectively, as described above with respect to Formula (II). In some embodiments, R^{17a} can be hydrogen, —(CH₂)—OH or —(CH₂)—OPG³. In some embodiments, B^{2a} can be an optionally substituted heterocyclic base, an optionally substituted heterocyclic base derivative, an optionally substituted protected heterocyclic base, or an optionally substituted protected heterocyclic base derivative, and if B^{2a} does not have one or more amino groups attached to a ring protected with one or more protecting groups and/or any —NH groups present in a ring of B^{2a} protected with one or more protecting groups, then B^{2a} can be B². When more than one protecting group is present on B^{2a}, the protecting groups can be the same or different.

Various protecting groups can be used to protect the oxygen of —(CH₂)—OH. In some embodiments, PG³ can be a triarylmethyl or levulinoyl protecting group. Suitable triarylmethyl protecting groups are described herein. By protecting the —(CH₂)—OH group, various undesirable side-reactions can be prevented or reduced which can make the separation of the desired compound can be less complex.

Various protecting groups can also be used to protect the optionally substituted heterocyclic base and/or optionally substituted heterocyclic base derivative. For example, one or more amino groups attached to a ring and/or any —NH groups present in a ring of the optionally substituted heterocyclic base and/or optionally substituted heterocyclic base derivative can be protected with one or more suitable protecting groups. In an embodiment, the optionally substituted heterocyclic base and/or optionally substituted heterocyclic base derivative can be protected with one or more triarylmethyl protecting groups. Examples of triarylmethyl protecting groups are disclosed herein.

As shown in Scheme 2b, diphenylphosphite can be reacted with

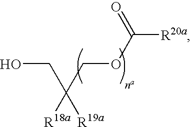

a nucleoside of having the structure

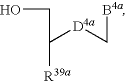

an amino acid, and a suitable oxidizing agent to form a compound of Formula (B).

A variety of amino acids can be used. In some embodiments, the amino acid can have the following structure

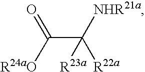

wherein R^{21a}, R^{22a}, R^{23a} and R^{24a} can be the same as R²¹, R²², R²³ and R²⁴, as described herein with respect to Formula (II).

Suitable oxidizing agents are known to those skilled in the art. In an embodiment, the oxidizing agent can be carbon tetrachloride (CCl₄). In some embodiments, the oxidizing agent, such as CCl₄, oxidizes the phosphorus from (III) to (V).

If R^{17a} is —(CH₂)—OPG³, the protecting group, PG³, can be removed, and if there are any protecting groups present on the optionally substituted heterocyclic base or optionally substituted heterocyclic base derivative, the protecting group(s) can be removed with one or more suitable reagents. For example, when the protecting group(s) is/are triarylmethyl protecting group(s) on B^{2a}, the protecting group(s) can be removed with an acid. In an embodiment, the acid can be acetic acid. When PG³ is a levulinoyl group, in some embodiments, PG³ can be removed using hydrazinium acetate. When PG³ is a triarylmethyl protecting group, in some embodiments, PG³ can be removed using an acid such as acetic acid. In some embodiments, the protecting groups can be removed sequentially. For example, in an embodiment, the protecting group(s), PG³ attached can be removed before any protecting groups present on the optionally substituted heterocyclic base or optionally substituted heterocyclic base derivative. In other embodiments, the protecting group(s), PG³ attached can be removed after any protecting groups present on the optionally substituted heterocyclic base or optionally substituted heterocyclic base derivative. In still other embodiments, the protecting groups can be removed almost simultaneously. If R^{17a} and B^{2a} are not protected, the compound of Formula B can be a compound of Formula (II).

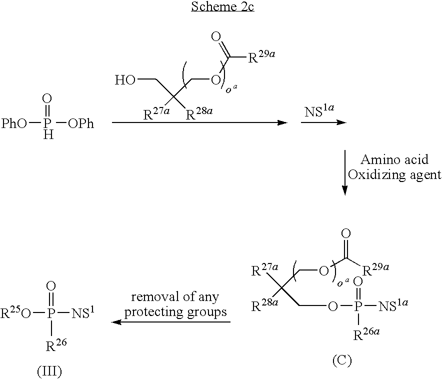

An embodiment disclosed herein relates to a method of synthesizing a compound of Formula (III) that can include the transformations shown in Scheme 2c. As shown in Scheme 2c, a compound of Formula (III) can be prepared in a similar manner to a compound of Formula (I). In Scheme 2b, R^{26a}, R^{27a}, R^{28a}, R^{29a} and o^{a} can be the same as R²⁶, R²⁷, R²⁸, R²⁹ and o, respectively, as described herein with respect to Formula (III).

As illustrated in Scheme 2c, diphenylphosphite can be reacted with

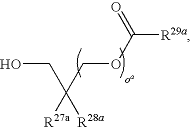

a nucleoside (NS^{1a}), an amino acid and an oxidizing agent to form a compound of Formula (C). As previously discussed, any suitable oxidizing agent can be used. In an embodiment, the oxidizing agent is carbon tetrachloride (CCl₄). In some embodiments, the oxidizing agent, such as CCl₄, oxidizes the phosphorus from (III) to (V).

Various amino acids can be used to form a compound of Formula (II). In some embodiments, the amino acid can have the structure:

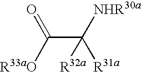

wherein R^{30a}, R^{31a}, R^{32a} and R^{33a} can be the same as R³⁰, R³¹, R³² and R³³, respectively, as described herein with respect to Formula (III).

In some embodiments, any oxygens present as hydroxy groups on the nucleoside, can be protected with suitable protecting groups. In an embodiment, any hydroxy groups attached to the 2′- and/or 3′-carbons can be protected with one or more suitable protecting groups. In an embodiment, when one or more hydroxy groups are attached to the 2′- and/or 3′-carbons, the oxygens of any hydroxy groups can be protected with one or more levulinoyl groups.

In some embodiments, the optionally substituted heterocyclic base or optionally substituted heterocyclic base derivative part of the nucleoside can be protected with one or more suitable protecting groups. In an embodiment, one or more amino groups attached to a ring and/or any —NH groups present in a ring of the optionally substituted heterocyclic base and/or optionally substituted heterocyclic base derivative can be protected with one or more suitable protecting groups. In some embodiments, the protecting group(s) can be triarylmethyl protecting group(s), such as those described herein. If one or more protecting groups are present (for example, attached to the 2′- and/or 3′-carbons of the nucleoside and/or on the optionally substituted heterocyclic base or the optionally substituted heterocyclic base derivative) the protecting group(s) can be removed to obtain a compound of Formula (III). As previously discussed, the protecting groups can be removed sequentially or simultaneously. If there are no protecting groups present on the compound of Formula (C), then the compound of Formula (C) can be a compound of Formula (III).

Various nucleosides can be used, including those described herein. In some embodiments, the nucleoside, NS^{1a}, can have the formula,

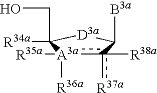

wherein A^{3a}, B^{3a}, D^{3a}, R^{34a}, R^{35a} and R^{38a} can be the same as A³, B³, D³, R³⁴, R³⁵ and R³⁸, respectively, as described above with respect to Formula (IV). In some embodiments, R^{36a} can be absent or selected from hydrogen, halogen, azido, amino, hydroxy, an —O-linked amino acid and O-PG⁴, wherein if R^{36a} is not a hydroxy group, then R^{36a} can be R³⁶. In some embodiments, R^{37a} can be selected from absent or selected from hydrogen, halogen, hydroxy, —CN, —NC, an optionally substituted C₁₋₄ alkyl, an optionally substituted C₁₋₄ alkoxy, an —O-linked amino acid and O-PG⁵, wherein if R^{37a} is not a hydroxy group, then R^{37a} can be R³⁷. Various protecting groups can be used for PG⁴ and PG⁵. In some embodiments, PG⁴ can be a triarylmethyl or levulinoyl protecting group. In some embodiments, PG⁵ can be a triarylmethyl or levulinoyl protecting group. In an embodiment, PG⁴ and PG⁵ can both be levulinoyl protecting groups. As discussed previously, by protecting the oxygens on the 2′- and 3′-carbons, various undesirable side-reactions can be prevented or reduced. This can make separation of the desired compound less complicated.

The optionally substituted heterocyclic base and/or optionally substituted heterocyclic base derivative, B^{3a}, can also be protected with one or more suitable protecting groups. For example, one or more amino groups attached to a ring and/or any —NH groups present in a ring of the optionally substituted heterocyclic base and/or optionally substituted heterocyclic base derivative. If the amino groups attached to the ring and the —NH groups present in the ring of the optionally substituted heterocyclic base and/or the optionally substituted heterocyclic base derivative are not protected, then B^{3a} can be B³. In an embodiment, the optionally substituted heterocyclic base and/or optionally substituted heterocyclic base derivative can be protected with more or more triarylmethyl protecting groups. Examples of suitable triarylmethyl protecting groups are described herein. Any protecting groups that are present on the compound of Formula (C) can be removed using similar methodology as described with respect to a compound of Formula (A). If there are no protecting groups present on the compound of Formula (C) when NS^{1a} is

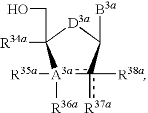

then the compound of Formula (C) can be a compound of Formula (III) with the nucleoside portion having the structure of a compound of Formula (IV).

In other embodiments, the nucleoside, NS^{1a}, can have the formula,

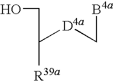

wherein D^{4a} can be the same as D⁴ as described above with respect to Formula (V). The variable R^{39a} can be hydrogen, —(CH₂)—OH or —(CH₂)—OPG⁶ in which PG⁶ denotes an appropriate protecting group. A non-limiting list of suitable protecting groups include triarylmethyl protecting groups and levulinoyl. When PG⁶ is a levulinoyl group, the levulinoyl group can be removed with hydrazinium acetate. If PG⁶ is a triarylmethyl protecting group, PG⁶ can be removed with an acid (e.g., acetic acid). can be selected from an optionally substituted heterocyclic base, an optionally substituted heterocyclic base derivative, a protected optionally substituted heterocyclic base and a protected optionally substituted heterocyclic base derivative. When B^{4a} is a protected optionally substituted heterocyclic base or a protected optionally substituted heterocyclic base derivative, one or more amino groups attached to a ring and/or any —NH groups present in a ring of the optionally substituted heterocyclic base and/or optionally substituted heterocyclic base derivative can be protected with one or more suitable protecting groups. In some embodiments, B^{4a} can include one or more protecting groups on the one or more amino groups attached to a ring and/or any —NH groups present in a ring of the optionally substituted heterocyclic base and/or optionally substituted heterocyclic base derivative. In an embodiment, B^{4a} can include one or more triarylmethyl protecting groups. Methods for removing protecting groups are well known to those skilled in the art. For example, when the protecting group is a triarylmethyl group, it can be removed with an acid such as acetic acid. If there are no protecting groups present in B^{4a}, then B^{4a} can be B⁴. If the nucleoside does not have protecting group(s), a compound of Formula (C) can be a compound of Formula (III) in which the nucleoside portion has the structure of a compound of Formula (V).

The methods of synthesis described above in Schemes 2a, 2b and 2c can be used to synthesize any protected nucleotide analogs of Formulae (I), (II) and (III) and any embodiments of Formulae (I), (II) and (III) described herein.

The 2,2-disubstituted-acyl(oxyalkyl) groups disclosed herein, such as

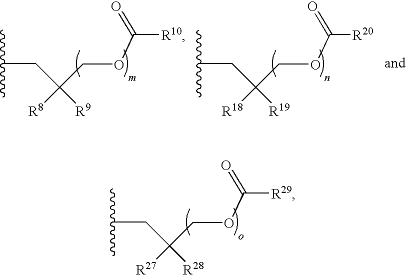

and an amino acid, such as

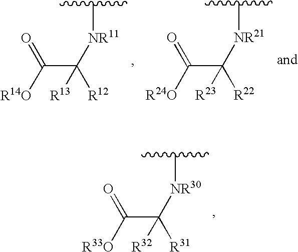

can be introduced into a polynucleotide, an oligonucleotide, or an analog thereof using methods known to those skilled in the art.

### Pharmaceutical Compositions

An embodiment described herein relates to a pharmaceutical composition, that can include a therapeutically effective amount of one or more compounds described herein (e.g., a compound of Formula (I), a compound of Formula (II) and/or a compound of Formula (III)) and a pharmaceutically acceptable carrier, diluent, excipient or combination thereof.

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, intramuscular, intraocular, intranasal, intravenous, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical compositions will generally be tailored to the specific intended route of administration.

The term “physiologically acceptable” defines a carrier, diluent or excipient that does not abrogate the biological activity and properties of the compound.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Suitable routes of administration may, for example, include oral, rectal, topical transmucosal, or intestinal administration; parenteral delivery, including intramuscular, subcutaneous, intravenous, intramedullary injections, as well as intrathecal, direct intraventricular, intraperitoneal, intranasal, intraocular injections or as an aerosol inhalant.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes will be targeted to and taken up selectively by the organ.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that include a compound disclosed herein formulated in a compatible pharmaceutical carrier may also be prepared, placed in an appropriate container, and labeled for treatment of an indicated condition.

### Methods of Use

One embodiment disclosed herein relates to a method of treating and/or ameliorating a disease or condition that can include administering to a subject a therapeutically effective amount of one or more compounds described herein, such as a compound of Formula (I), a compound of Formula (II) and/or a compound of Formula (III), or a pharmaceutical composition that includes a compound described herein.

Some embodiments disclosed herein relate to a method of ameliorating or treating a neoplastic disease that can include administering to a subject suffering from the neoplastic disease a therapeutically effective amount of one or more compounds described herein (e.g., a compound of Formula (I), a compound of Formula (II) and/or a compound of Formula (III)) or a pharmaceutical composition that includes one or more compounds described herein. In an embodiment, the neoplastic disease can be cancer. In some embodiments, the neoplastic disease can be a tumor such as a solid tumor. In an embodiment, the neoplastic disease can be leukemia. Examples of leukemias include, but are not limited to, acute lymphoblastic leukemia (ALL), acute myeloid leukemia (AML) and juvenile myelomonocytic leukemia (JMML).

An embodiment disclosed herein relates to a method of inhibiting the growth of a tumor that can include administering to a subject having the tumor a therapeutically effective amount of one or more compounds described herein or a pharmaceutical composition that includes one or more compounds described herein.

Other embodiments disclosed herein relates to a method of ameliorating or treating a viral infection that can include administering to a subject suffering from the viral infection a therapeutically effective amount of one or more compounds described herein or a pharmaceutical composition that includes one or more compounds described herein. In an embodiment, the viral infection can be caused by a virus selected from an adenovirus, an Alphaviridae, an Arbovirus, an Astrovirus, a Bunyaviridae, a Coronaviridae, a Filoviridae, a Flaviviridae, a Hepadnaviridae, a Herpesviridae, an Alphaherpesvirinae, a Betaherpesvirinae, a Gammaherpesvirinae, a Norwalk Virus, an Astroviridae, a Caliciviridae, an Orthomyxoviridae, a Paramyxoviridae, a Paramyxoviruses, a Rubulavirus, a Morbillivirus, a Papovaviridae, a Parvoviridae, a Picornaviridae, an Aphthoviridae, a Cardioviridae, an Enteroviridae, a Coxsackie virus, a Polio Virus, a Rhinoviridae, a Phycodnaviridae, a Poxyiridae, a Reoviridae, a Rotavirus, a Retroviridae, an A-Type Retrovirus, an Immunodeficiency Virus, a Leukemia Viruses, an Avian Sarcoma Viruses, a Rhabdoviruses, a Rubiviridae and/or a Togaviridae. In an embodiment, the viral infection is a hepatitis C viral infection. In another embodiment, the viral infection is a HIV infection.

One embodiment disclosed herein relates to a method of ameliorating or treating a parasitic disease that can include administering to a subject suffering from the parasitic disease a therapeutically effective amount of one or more compounds described herein or a pharmaceutical composition that includes one or more compounds described herein. In an embodiment, the parasite disease can be Chagas' disease.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

The term “therapeutically effective amount” is used to indicate an amount of an active compound, or pharmaceutical agent, that elicits the biological or medicinal response indicated. For example, a therapeutically effective amount of compound can be the amount need to prevent, alleviate or ameliorate symptoms of disease or prolong the survival of the subject being treated This response may occur in a tissue, system, animal or human and includes alleviation of the symptoms of the disease being treated. Determination of a therapeutically effective amount is well within the capability of those skilled in the art, especially in light of the detailed disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. (See e.g., Fingl et al. 1975, in “The Pharmacological Basis of Therapeutics”, which is hereby incorporated herein by reference in its entirety, with particular reference to Ch. 1, p. 1). The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the patient. In some embodiments, the compounds will be administered for a period of continuous therapy, for example for a week or more, or for months or years.

In instances where human dosages for compounds have been established for at least some condition, those same dosages, or dosages that are between about 0.1% and 500%, more preferably between about 25% and 250% of the established human dosage will be used. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Alternatively dosages may be based and calculated upon the surface area of the patient, as understood by those of skill in the art.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

### EXAMPLES

Additional embodiments are disclosed in further detail in the following examples, which are not in any way intended to limit the scope of the claims.

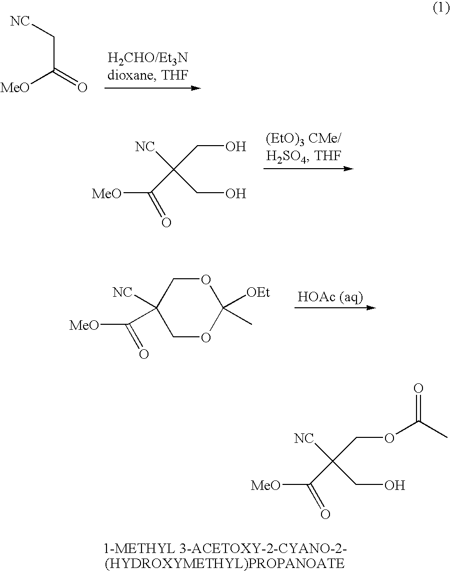

Methyl 2-cyano-3-hydroxy-2-hydroxymethylpropanoate. Formaldehyde (66.7 mmol, 2.0 g) was added as 20% aq solution (10 g) to 1,4-dioxane (30 mL) on an ice-bath. Methyl cyanoacetate (30.3 mmol, 2.12 mL) and Et₃N (0.61 mmol, 0.61 mL of 1 mol L⁻¹ solution in THF) were added and the mixture was stirred for 20 min. Another portion of Et₃N (0.61 mmol) was added and the ice-bath was removed. The mixture was stirred for 1.5 h at room temperature. The mixture was then diluted with water (200 mL) and extracted with benzene (3×50 mL) to remove side products. The aqueous phase was evaporated under reduced pressure at 30° C. to one fourth of the original volume and extracted 5 times with ethyl acetate. The combined extracts were dried over Na₂SO₄ and evaporated to a clear oil. The yield was 72% (4.82 g). The compound was used without characterization to the next step.

Methyl 5-cyano-2-ethoxy-2-methyl-1,3-dioxane-5-carboxylate. Methyl 2-cyano-3-hydroxy-2-hydroxymethylpropanoate (23.3 mmol, 3.7 g) was dissolved in dry THF (8 mL) and triethyl orthoacetate (34.9 mmol, 6.55 mL) was added. A catalytic amount of concentrated sulfuric acid (0.70 mmol, 37 μL) was added and the mixture was stirred over night at room temperature. The mixture was poured into a stirred ice-cold aq. NaHCO₃ (5%, 50 mL). The product was extracted into Et₂O (2×50 mL) and the extracts were washed with saturated aq. NaCl and dried over Na₂SO₄. The solvent was evaporated and purified by Silica gel chromatography applying a stepwise gradient from 5% ethyl acetate in dichloromethane to pure ethyl acetate. The product was obtained in 42% yield (5.33 g) as a clear oil that started to crystallize ¹H NMR for the major diastereomer (CDCl₃) 4.34 (d, J=7.0 Hz, 2H, —CH₂O—), 4.03 (d, J=8.5 Hz, 2H, —CH₂O—), 3.84 (s, 3H, OMe), 3.54 (q, J=7.2 Hz, 2H, —CH₂CH₃), 1.55 (s, 3H, —CH₃), 1.25 (t, J=7.2, 3H, —CH₂CH₃). ¹³C NMR for the major diastereomer (CDCl₃) 164.8 (C═O), 117.0 (CN), 111.4 (C2), 62.3 (C4 and C6), 59.1 (—CH₂CH₃), 53.9 (—OCH₃), 42.4 (C5), 22.3 (2-CH₃), 15.0 (CH₂CH₃).

Methyl 3-acetyloxy-2-cyano-2-(hydroxymethyl)propanoate. Methyl 5-cyano-2-ethoxy-2-methyl-1,3-dioxane-5-carboxylate (2.18 mmol, 0.50 g) was dissolved in a mixture of acetic acid and water (4:1, v/v, 20 mL) and the mixture was stirred for 2 h at room temperature, after which the mixture was evaporated to dryness and the residue was coevaporated 3 times with water. The product was purified by Silica gel chromatography, eluting with dichloromethane containing 5% MeOH. The yield was 52% (0.23 g). ¹H NMR (CDCl₃) 4.53 (d, J=11.0 Hz, 1H, —CH₂OAc), 4.50 (d, J=11.0 Hz, 1H, —CH₂OAc), 4.04 (d, J=6.5 Hz, 2H, —CH₂OH), 3.91 (s, 3H, —OMe), 2.90 (t, J=6.5 Hz, —OH), 2.16 (s, 3H, —C(O)CH₃). ¹³C NMR (CDCl₃) 170.4 (C═O), 166.0 (C═O), 116.0 (CN), 63.1 (—CH₂OH), 62.3 (—CH₂OAc), 54.1 (—OMe), 51.0 (C2), 20.6 (—C(O)CH₃).

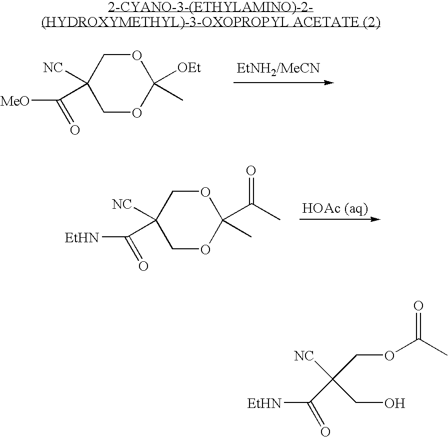

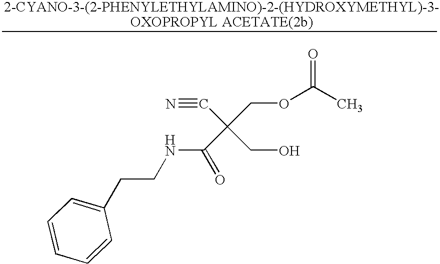

2-cyano-3-(2-phenylethylamino)-2-(hydroxymethyl)-3-oxopropyl acetate was prepared according to the procedure described in Poijärvi, P.; Maki, E.; Tomperi, J.; Ora, M.; Oivanen, M.; Lönnberg, H., Helve. Chim. Acta. (2002) 85, 1869-1876, which is hereby incorporated by reference for the limited purpose of describing the method of synthesizing and purifying 2-cyano-3-(2-phenylethylamino)-2-(hydroxymethyl)-3-oxopropyl acetate.

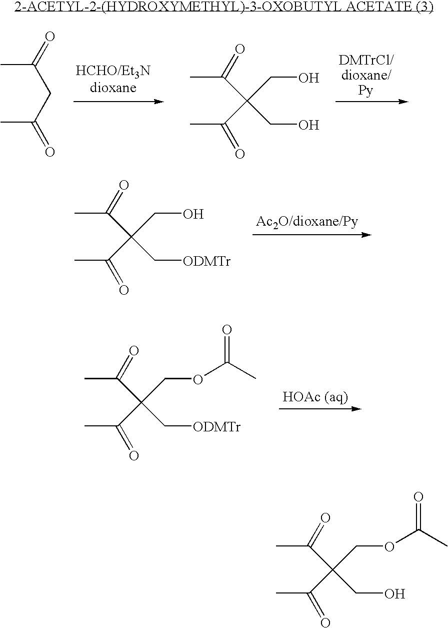

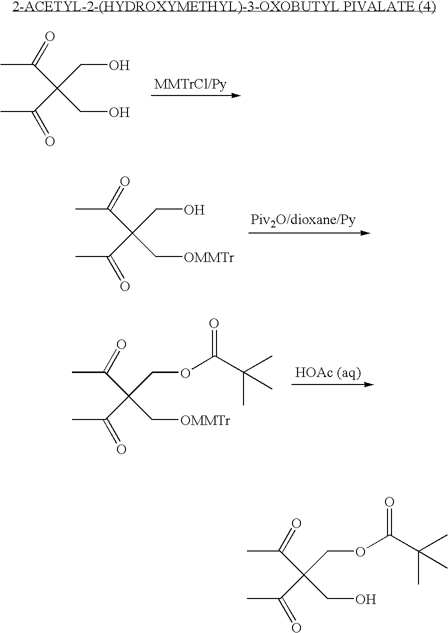

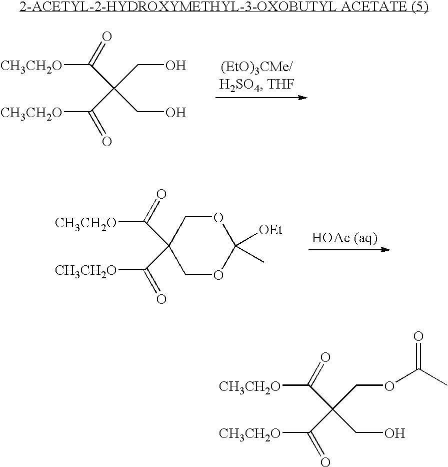

Diethyl 2-ethoxy-2-methyl-1,3-dioxane-5,5-dicarboxylate. Concentrated H₂SO₄ (1.3 mmol; 71 μL) was added to a mixture of diethyl 2,2-bis(hydroxymethyl)malonate (43.5 mmol, 9.6 g) and triethyl orthoacetate (65.2 mmol; 11.9 mL) in dry THF (15 mL). The reaction was allowed to proceed overnight and the mixture was the poured into an ice-cold solution of 5% NaHCO₃ (50 mL). The product was extracted with diethyl ether (2×50 mL), washed with saturated aqueous NaCl (2×50 mL) and dried over Na₂SO₄. The solvent was evaporated and the crude product was purified on a silica gel column eluting with a mixture of dichloromethane and methanol (95:5, v/v). The product was obtained as clear oil in 89% yield (11.3 g). ¹H NMR δ_{H} (500 MHz, CDCl₃): 4.30-4.36 (m, 6H, 4-CH₂, 6-CH₂ and 5-COOCH₂Me), 4.18 (q, J=7.1 Hz, 5-COOCH₂Me), 3.54 (q, J=7.10 Hz, 2H, 2-OCH₂Me), 1.46 (s, 3H, 2-CH₃), 1.32 (t, J=7.10 Hz, 3H, 2-OCH₂Me), 1.27 (t, J=7.1 Hz 3H, 5-COOCH₂Me), 1.26 (t, J=7.1 Hz 3H, 5-COOCH₂Me). ¹³C NMR (500 MHz, CDCl₃): δ=168.0 and 167.0 (5-COOEt), 111.1 (C2), 62.0 and 61.9 (5-COOCH₂Me), 61.6 (C4 and C6), 58.7 (2-OCH₂Me), 52.3 (C5), 22.5 (2-Me), 15.1 (2-OCH₂CH₃), 14.0 and 13.9 (5-COOCH₂CH₃).

Diethyl 2-(acetyloxymethyl)-2-(hydroxymethyl)malonate. Diethyl 2-ethoxy-2-methyl-1,3-dioxane-5,5-dicarboxylate (17.9 mmol; 5.2 g) was dissolved in 80% aqueous acetic acid (30 mL) and left for 2 h at room temperature. The solution was evaporated to dryness and the residue was coevaporated three times with water. The product was purified by silica gel column chromatography eluting with ethyl acetate in dichloromethane (8:92, v/v). The product was obtained as yellowish oil in 75% yield (3.6 g). ¹H NMR δ_{H} (500 MHz, CDCl₃): 4.76 (s, 2H, CH₂OAc), 4.26 (q, J=7.10 Hz, 4H, OCH₂Me), 4.05 (d, J=7.10 Hz, 2H, CH₂OH), 2.72 (t, J=7.1 Hz, 1H, CH₂OH), 2.08 (s, 3H, Ac), 1.27 (t, J=7.10 Hz, 6H, OCH₂CH₃). ¹³C NMR (500 MHz, CDCl₃): δ=170.9 (C═O Ac), 168.1 (2×C═O malonate), 62.3 and 62.2 (CH₂OH and CH₂OAc), 61.9 (2×OCH₂CH₃) δ9.6 (spiro C), 20.7 (CH₃ Ac), 14.0 (2×OCH₂CH₃).

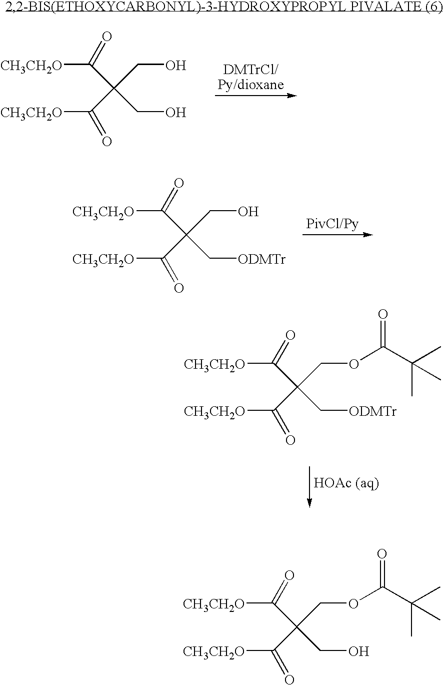

2,2-Bis(ethoxycarbonyl)-3-(4,4′-dimethoxytrityloxy)propyl pivalate. Diethyl 2,2-bis(hydroxymethyl)malonate was reacted with 1 equiv. of 4,4′-dimethoxytrityl chloride in 1,4-dioxane containing 1 equivalent of pyridine. Diethyl 2-(4,4′-dimethoxytrityloxymethyl)-2-(hydroxymethyl)malonate (2.35 g, 4.50 mmol) was acylated with pivaloyl chloride (0.83 mL, 6.75 mmol) in dry MeCN (10 mL) containing 3 equivalent pyridine (1.09 mL, 13.5 mmol). After 3 days at room temperature, the reaction was quenched with MeOH (20 mL) and a conventional CH₂Cl₂/aq HCO₃⁻ workup was carried out. Silica gel chromatography (EtOAc/hexane 1:1, v/v) gave 2.47 g (90%) of the desired product as yellowish syrup. ¹H NMR (CDCl₃, 200 MHz): 7.13-7.39 [m, 9H, (MeO)₂ Tr]; 6.81 (d, 4H, [MeO]₂ Tr); 4.71 (s, 2H, CH₂OPiv); 4.15 (q, J=7.1, 4H, OCH₂CH₃); 3.78 [s, 6H, (CH₃O)₂Tr]; 3.67 (s, 2H, CH₂ODMTr); 1.27 (t, J=7.1, 6H, OCH₂CH₃); 1.02 [s, 9H, COC(CH₃)₃].

2,2-Bis(ethoxycarbonyl)-3-hydroxypropyl pivalate. 2,2-Bis(ethoxycarbonyl)-3-(4,4′-dimethoxytrityloxy)propyl pivalate (2.47 g, 4.07 mmol) in a 4:1 mixture of CH₂Cl₂ and MeOH (20 mL) was treated for 4 h at room temperature with TFA (2.00 mL, 26.0 mmol) to remove the dimethoxytrityl group. The mixture was neutralized with pyridine (2.30 mL, 28.6 mmol), subjected to CH₂Cl₂/aq workup and purified by silica gel chromatography (EtOAc/hexane 3:7, v/v) to obtain 1.15 g (93%) of the desired product. ¹H NMR (CDCl₃, 200 MHz): 4.59 (s, 2H, CH₂OPiv); 4.25 (q, J=7.1, 4H, OCH₂CH₃); 4.01 (s, 2H, CH₂OH); 1.28 (t, J=7.1, 6H, OCH₂CH₃); 1.18 [s, 9H, COC(CH₃)_{3]}. ESI− MS⁺: m/z 305.4 ([MH]⁺), 322.6 ([MNH₄]⁺), 327.6 ([MNa]⁺), 343.5 ([MK]⁺).

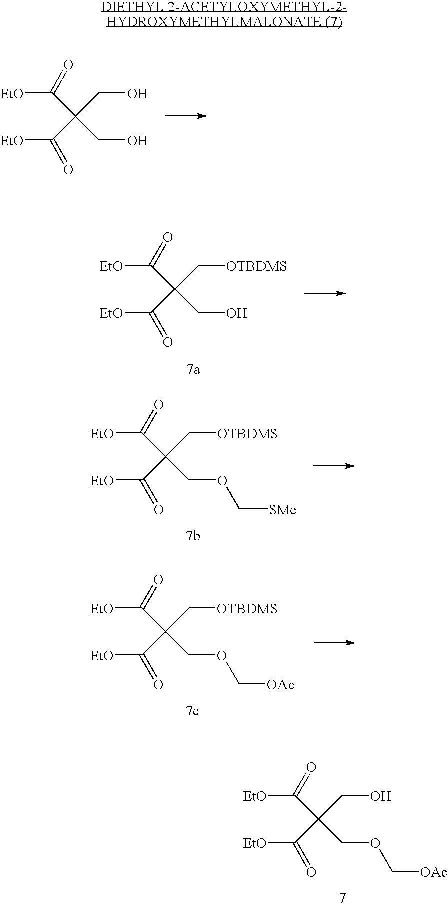

Diethyl 2-(tert-butyldimethylsilyloxymethyl)-2-hydroxymethylmalonate (7a). Diethyl 2,2-bis(hydroxymethyl)malonate (28.3 mmol; 6.23 g) was coevaporated twice from dry pyridine and dissolved in the same solvent (20 mL). tert-Butyldimethylsilyl chloride (25.5 mmol; 3.85 g) in dry pyridine (10 mL) was added portionwise. The reaction was allowed to proceed for 4 days. The mixture was evaporated to a solid foam, which was then equilibrated between water (200 mL) and DCM (4×100 mL). The organic phase was dried on Na₂SO₄. The product was purified by silica gel chromatography eluting with 10% ethyl acetate in DCM. The yield was 78%. ¹H NMR (CDCl₃) δ 4.18-4.25 (m, 4H, OCH₂Me), 4.10 (s, 2H, CH₂OSi), 4.06 (s, 2H, CH₂OH), 2.63 (br s, 1H, OH), 1.26 (t, J=7.0 Hz, 6H, OCH₂CH₃), 0.85 (s, 9H, Si—SMe₃), 0.05 (s, 6H, Me-Si). ¹³C NMR (CDCl₃) δ 169.2 (C═O), 63.3 (CH₂OH), 62.8 (CH₂OSi), 61.6 (spiro C), 61.4 (OCH₂Me), 25.6 [C(CH₃)₃], 18.0 (Si—CMe₃), 14.0 (OCH₂CH₃), −3.6 (Si—CH₃). MS [M+H]⁺ obsd. 335.7, calcd. 335.2; [M+Na] obsd. 357.6, calcd. 357.2.

Diethyl 2-(tert-butyldimethylsilyloxymethyl)-2-methylthiomethylmalonate (7b). Compound 7a (19.7 mmol; 6.59 g) was dissolved into a mixture of acetic anhydride (40 mL), acetic acid (12.5 mL) and DMSO (61 mL) and the mixture was stirred overnight. The reaction was stopped by dilution with cold aq. Na₂CO₃ (290 ml 10% aq. solution) and the product was extracted in diethyl ether (4×120 mL). The combined organic phase was dried on Na₂SO₄. The product was purified by silica gel chromatography using DCM as an eluent. The yield was 91%. ¹H NMR (CDCl₃) δ 4.61 (s, 2H, OCH₂S), 4.14-4.19 (m, 4H, OCH₂Me), 4.06 (s, 2H, CH₂OSi), 4.00 (s, 2H, CH₂OCH₂SMe), 2.06 (SCH₃), 1.22 (t, J=7.0 Hz, 6H, OCH₂CH₃), 0.83 (s, 9H, Si—SMe₃), 0.02 (s, 6H, Me-Si). ¹³C NMR (CDCl₃) δ 168.3 (C═O), 75.6 (CH₂S), 65.7 (CH₂OCH₂SMe), 61.4 (CH₂OSi), 61.2 (spiro C), 60.9 (OCH₂Me), 25.6 [C(CH₃)₃], 18.0 (Si—CMe₃), 14.0 (OCH₂CH₃), 13.7 (SCH₃), −3.6 (Si—CH₃). MS [M+H]⁺ obsd. 395.4, calcd. 395.2; [M+Na]⁺ obsd. 417.6, calcd. 417.2.

Diethyl 2-acetyloxymethyl-2-(tert-butyldimethylsilyloxymethyl)malonate (7c). Compound 7b (17.9 mmol; 7.08 g) was dissolved in dry DCM (96 mL) under nitrogen. Sulfurylchloride (21.5 mmol; 1.74 mL of 1.0 mol L⁻¹ solution in DCM) was added in three portions and the mixture was stirred for 70 min under nitrogen. The solvent was removed under reduced pressure and the residue was dissolved into dry DCM (53 mL). Potassium acetate (30.9 mmol; 3.03 g) and dibenzo-18-crown-6 (13.5 mmol; 4.85 g) in DCM (50 mL) were added and the mixture was stirred for one hour and a half. Ethyl acetate (140 mL) was added, the organic phase was washed with water (2×190 mL) and dried on Na₂SO₄. The product was purified by silica gel chromatography using DCM as an eluent. The yield was 71%. ¹H NMR (CDCl₃) δ 5.24 (s, 2H, OCH₂O), 4.15-4.22 (m, 4H, OCH₂Me), 4.13 (s, 2H, CH₂OSi), 4.08 (s, 2H, CH₂OAc), 2.08 (Ac), 1.26 (t, J=8.0 Hz, 6H, OCH₂CH₃), 0.85 (s, 9H, Si—SMe₃), 0.04 (s, 6H, Me-Si). ¹³C NMR (CDCl₃) δ 170.2 (Ac), 168.0 (C═O), 89.3 (OCH₂O), 67.5 (CH₂OAc), 61.4 (OCH₂Me), 61.1 (CH₂OSi), 60.2 (spiro C), 25.6 [C(CH₃)₃], 21.0 (Ac), 18.1 (Si—CMe₃), 14.0 (OCH₂CH₃), −5.7 (Si—CH₃). MS [M+Na]⁺ obsd. 429.6, calcd. 429.2.

Diethyl 2-acetyloxymethyl-2-hydroxymethylmalonate (7). Compound 7c (7.2 mmol; 2.93 g) was dissolved in dry THF (23 mL) and triethylamine trihydrogenfluoride (8.64 mmol; 1.42 mL) was added. The mixture was stirred for one week. Aq. triethylammonium acetate (13 mL of 2.0 mol L⁻¹ solution) was added. The mixture was evaporated to dryness and the residue was purified by silica gel chromatography using DCM containing 2-5% MeOH as an eluent. The yield was 74%. ¹H NMR (CDCl₃) δ 5.25 (s, 2H, OCH₂O), 4.16-4.29 (m, 6H, OCH₂Me and CH₂OAc), 4.13 (s, 2H, CH₂OH), 2.10 (Ac), 1.81 (br s, 1H, OH), 1.26 (t, J=9.0 Hz, 6H, OCH₂CH₃). MS [M+Na]⁺ obsd. 315.3, calcd. 315.1.

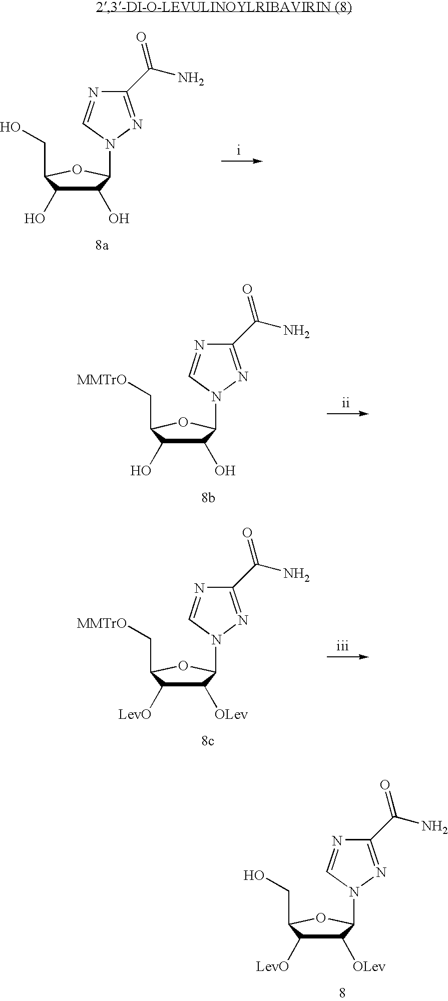

5′-O-(4-Methoxytrityl)ribavirin (8b). Ribavirin (compound 8a; 8.31 mmol; 2.03 g) was dried by repeated coevaporations from dry pyridine and dissolved in the same solvent (15 mL). 4-Methoxytrityl chloride (8.32 mmol; 2.57 g) was added and the reaction was allowed to proceed overnight. The mixture was evaporated to dryness and the residue was equilibrated between chloroform and water. The organic phase was dried on Na₂SO₄. The crude product was purified by silica gel chromatography using gradient elution from 5 to 10% MeOH in DCM. Yield 68%. ¹H NMR (CDCl₃) δ 8.45 (s, 1H, H5), 7.39-741 (m, 4H, MMTr), 7.27-7.30 (m, 2H, MMTr), 7.21-7.24 (m, 4H, MMTr), 7.15-7.18 (m, 2H, MMTr), 7.09 (br s, 1H, NH), 6.78-6.80 (m, 2H, MMTr), 6.43 (br s, 1H, NH), 5.98 (d, J=3.5 Hz, 1H, H1′), 4.79 (dd, J=3.5 and 4.7 Hz, 1H, H2′), 4.48 (dd, J=4.7 and 5.1, 1H, H3′), 4.31 (m, 1H, H4′), 3.73 (s, 3H, MeO-MMTr), 3.43 (dd, J=10.6 and 2.8 Hz; 1H, H5′), 3.31 (dd, 10.6 and 4.3 Hz, 1H, H5″). ¹³C NMR (CDCl₃) δ 161.3 (C═O), 158.6 (MMTr), 156.5 (C3), 144.6 (MMTr), 144.0 (C5), 136.3 (MMTr), 130.4 (MMTr), 128.3 (MMTr), 127.9 (MMTr), 127.0 (MMTr), 113.2 (MMTr), 92.9 (C1′), 86.7 (MMTr), 84.6 (C4′), 75.3 (C2′), 71.1 (C3′), 63.5 (C5′), 55.2 (MMTr).

2′,3′-Di-O-levulinoyl-5′-O-(4-methoxytrityl)ribavirin (8c). Levulinic acid (28.3 mmol; 3.29 g) was dissolved in dry dioxane and the solution was cooled to 0° C. on an ice bath. Dicyclohexylcarbodiimide (14.2 mmol; 2.93 g) was added portionwise during 1 h. Dicyclohexylurea crystallized was removed by filtration. The filtrate and dioxane washing of the precipitate (5 mL) were combined and mixed with the solution of compound 8b (dried on P₂O₅) in dry pyridine (15 mL). A catalytic amount of 4-dimethylaminopyridine was added and the reaction was allowed to proceed overnight. Volatiles were removed under reduced pressure and the residue was subjected to DCM/aq. NaHCO₃ work-up. The organic phase was dried on Na₂SO₄. The crude product (8c) was used in the next step. ¹H NMR (CDCl₃) δ 8.36 (s, 1H, H5), 7.42-7.44 (m, 4H, MMTr), 7.23-7.32 (m, 8H, MMTr), 6.78-6.80 (m, 2H, MMTr), 6.66 (br s, 1H, NH), 6.08 (d, J=4.9 Hz, 1H, H1′), 6.00 (dd, J=4.9 and 5.3 Hz, 1H, H2′), 5.69 (br s, 1H, NH), 5.63 (dd, J=4.1 and 5.3, 1H, H3′), 4.40 (m, 1H, H4′), 3.80 (s, 3H, MeO-MMTr), 3.47 (dd, J=10.6 and 2.8 Hz; 1H, H5′), 3.36 (dd, 10.8 and 4.3 Hz, 1H, H5″), 2.76-2.81 (m, 4H, Lev), 2.61-2.67 (m, 4H, Lev), 2.20 (s, 6H, Lev). ¹³C NMR (CDCl₃) δ 206.3 (2×C═O Lev), 171.6 (C═O Lev), 171.3 (C═O Lev), 160.3 (C═O), 158.7 (MMTr), 157.2 (C3), 144.7 (MMTr), 143.7 (C5), 136.0 (MMTr), 130.5 (MMTr), 128.4 (MMTr), 128.0 (MMTr), 127.2 (MMTr), 113.2 (MMTr), 89.9 (C1′), 87.2 (MMTr), 83.1 (C4′), 74.3 (C2′), 71.4 (C3′), 62.9 (C5′), 55.2 (MMTr), 37.8 (Lev), 37.7 (Lev), 29.8 (Lev), 29.7 (Lev), 27.6 (Lev), 27.5 (Lev).

2′,3′-Di-O-levulinoylribavirin (8). Compound 8c (7.17 mmol; 5.11 g) was treated with 80% aq. AcOH (100 mL) overnight. The mixture was evaporated to dryness and the residue was purified by silica gel chromatography using gradient elution from 5 to 10% MeOH in DCM. Yield from compound 8b was 66%. ¹H NMR (CDCl₃+CD₃OD) δ 8.60 (s, 1H, H5), 7.34 (s, 1H, NH), 6.08 (d, J=4.3 Hz, 1H, H1′), 5.71 (dd, J=4.3 and 5.2 Hz, 1H, H2′), 5.58 (dd, J=4.3 and 5.2, 1H, H3′), 5.32 (s, 1H, NH), 4.35 (m, 1H H4′), 3.91 (dd, J=12.7 and 2.4 Hz, 1H, H5′), 3.77 (dd, J=12.7 and 2.8 Hz, 1H, H5″), 2.77-2.82 (m, 4H, Lev), 2.60-2.67 (m, 4H, Lev), 2.21 (s, 3H, Lev), 2.19 (s, 3H, Lev). ¹³C NMR (CDCl₃+CD₃OD) δ 207.0 (C═O Lev), 171.9 (C═O Lev), 171.4 (C═O Lev), 161.0 (C═O), 157.0 (C3), 144.7 (C5), 90.4 (C1′), 84.7 (C4′), 75.1 (C2′), 71.1 (C3′), 61.0 (C5′), 37.7 (Lev), 37.6 (Lev), 29.8 (Lev), 29.7 (Lev), 27.5 (Lev), 27.4 (Lev).

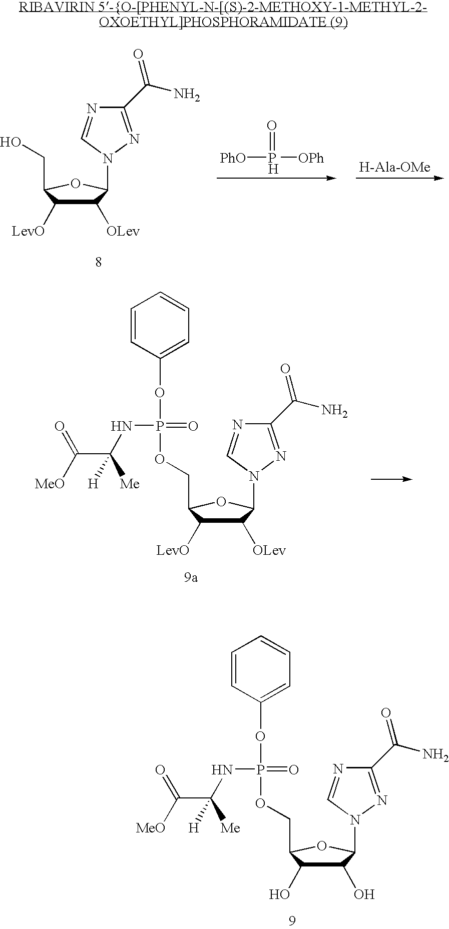

2′,3′-Di-O-levulinoylribavirin 5′-{O-[phenyl-N-[(S)-2-methoxy-1-methyl-2-oxoethyl]phosphoramidate (9a). Compound 8 (0.41 mmol; 0.18 g) was coevaporated twice from dry pyridine, dissolved in the same solvent (3.0 mL) and diphenylphosphite (0.61 mmol; 118 μL) was added under nitrogen. After 20 min, L-alanine methyl ester (0.86 mmol; 0.12 g) dried by coevaporation from pyridine was added dissolved in a mixture of dry MeCN (4.0 mL) and pyridine (1.0 mL). Immediately after this addition, CCl₄ (2.5 mL) and distilled triethylamine (2.8 mmol; 400 μL) were added. The reaction was allowed to proceed for 70 min and the mixture was then evaporated to dryness. Silica gel chromatography by a gradient elution from 3 to 10% of MeOH in DCM gave compound 9a as a foam in 67% yield. ¹H NMR (CDCl₃) mixture of R_{P} and S_{P} diastereomers δ 8.40 and 8.46 (2×s, 1H, H5), 7.34 and 7.37 (2×br s, 1H, NH₂), 7.10-7.30 (m, 5H, Ph), 6.38 and 6.46 (2×br s, 1H, NH₂), 6.09 and 6.10 (2×d, J=4.5 Hz, 1H, H1′), 5.71 and 5.73 (2×dd, J=4.5 and 5.0, 1H, H2′), 5.61 and 5.63 (2×dd, J=5.0 and 5.0, 1H, H3′), 4.45-4.49 (m, 1H, H4′), 4.28-4.43 (m, 2H, H5′ and H5″), 3.98-4.07 (m, 1H, H^{α}-Ala), 3.62 and 3.64 (2×s, 3H, MeO-Ala), 2.73-2.79 (m, 4H, Lev), 2.56-2.66 (m, 4H, Lev), 2.17 and 2.18 (2×s, 6H, Lev), 1.31 and 1.33 (2×d, J=7.2 Hz, 3H, Me Ala). ³¹P NMR (CDCl₃) 3.0 and 3.2.

Ribavirin 5′-{O-[phenyl-N-[(S)-2-methoxy-1-methyl-2-oxoethyl]phosphoramidate (9). Compound 9a (0.21 mmol; 0.14 g) was dissolved at 0° C. into a mixture of hydrazine hydrate (4.0 mmol; 124 μL), dry pyridine (4.0 mL) and AcOH (1.0 mL) and the reaction was allowed to proceed for 1 h. The unreacted hydrazine was quenched by acetone. The volatiles were removed under reduced pressure and the crude product was purified by silica gel chromatography increasing the MeOH content of DCM in a stepwise manner from 5% to 10% and then to 20%. Yield 60%. ¹H NMR (CD₃OD) mixture of R_{P} ans S_{P} diastereomers δ 8.72 and 8.74 (2×s, 1H, H5), 7.33-7.37 (m, 2H, Ph), 7.17-7.23 (m, 3H, Ph), 5.98 (2×d, J=3.5 Hz, 1H, H1′), 4.54 and 4.56 (2×dd, J=3.5 and 4.7 Hz, 1H, H2′), 4.47 (dd, J=4.7 and 5.9, 1H, H3′), 4.26-4.43 (m, 3H, H4′, H5′ and H5″), 3.91 and 3.94 (2×dd, J=9.3 and 7.2 Hz, H^{α}-Ala), 3.65 and 3.67 (2×s, 3H, MeO-Ala), 1.29 and 1.32 (2×d, J=7.2 Hz, 3H, Me Ala). ¹³C NMR (CD₃OD) δ 174.1 (C═O Ala), 161.9 (CONH₂), 157.1 (Ph), 150.7 (C3), 145.3 (C5), 92.4 (C1′), 83.1 (C4′), 74.8 (C2′), 70.2 (C3′), 65.9 (C5′), 51.9 (MeO-Ala), 49.8 (C^{α}-Ala), 19.1 (Me Ala). ³¹P NMR (CD₃OD) 3.8 and 4.0. HRMS: [M+H]⁺ obsd. 486.1389, calcd. 486.1384; [M+Na]⁺ obsd. 508.1206, calcd. 508.1204; [M+K]⁺ obsd. 524.0937, calcd. 524.0943.

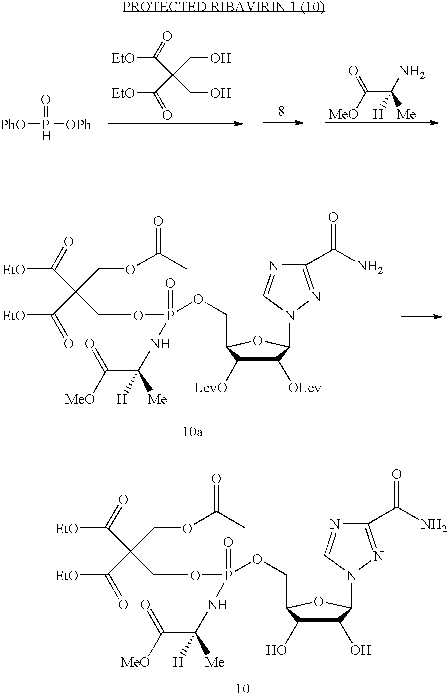

2′,3′-Di-O-levulinoylribavirin 5′-{O-[3-acetyloxy-2,2-bis(ethoxycarbonyl)propyl]-N-[(S)-2-methoxy-1-methyl-2-oxoethyl]phosphoramidate (10a). Diphenylphosphite was dissolved into dry pyridine (1.0 mL) and diethyl 2-acetyloxymethyl-2-hydroxymethylmalonate (0.29 mmol; 56 μL) in dry pyridine (1 mL) was added positionwise. After 30 min, compound 8 (0.34 mmol; 0.151 g) in dry pyridine (1.5 mL) was added dropwise and the reaction was allowed to proceed for 2 h. L-Alanine methyl ester (0.29 mmol; 41 mg) in dry pyridine (250 μL) was added, followed by dry MeCN (3.5 mL), CCl₄ (1.8 mL) and triethyl amine (1.45 mmol; 205 μL). The reaction was allowed to proceed for 45 min and the volatiles were then removed under reduced pressure. Crude compound 10a was purified by silica gel chromatography using gradient elution from 3 to 15% MeOH in DCM. Yield 44%. ¹H NMR (CDCl₃) mixture of R_{P} ans S_{P} diastereomers δ 8.41 and 8.46 (2×s, 1H, H5), 740 and 7.46 (2×br s, 1H, NH₂), 6.06 and 6.08 (2×d, J=4.5 Hz, 1H, H1′), 5.89 (br s, 1H, NH₂), 5.71 and 5.73 (2×dd, J=4.5 and 4.7 Hz, 1H, H2′), 5.56 and 5.58 (2×dd, J=4.7 and 4.9 Hz, 1H, H3′), 4.43-4.58 (m, 5H, H4′, CH₂OAc and CH₂OP), 4.18-4.36 (m, 6H, H5′, H5″, CH₂CH₃), 3.90-3.96 (m, H^{α}-Ala), 3.71 and 3.73 (2×s, 3H, MeO-Ala), 2.76-2.83 (m, 4H, Lev), 2.57-2.68 (m, 4H, Lev), 2.18 and 2.19 (2×s, 3H, Lev), 2.20 and 2.21 (2×s, 3H, Lev), 2.05 and 2.06 (2×s, 3H, Ac), 1.84 (br s, 1H, NH—P), 1.35 (d, J=7.0 Hz, 3H, Me Ala), 1.24-1.27 (m, 6H, CH₂CH₃). ³¹P NMR (CDCl₃)=7.2 and 7.4.

Ribavirin 5′-{O-[3-acetyloxy-2,2-bis(ethoxycarbonyl)propyl]-N-[(S)-2-methoxy-1-methyl-2-oxoethyl]phosphoramidate (10). Compound 10a (0.36 mmol; 0.31 g) was added into a mixture of hydrazine hydrate (3.98 mmol; 124 μL), pyridine (4.0 mL) and AcOH (1.0 mL). After 30 min, acetone was added to quench the unreacted hydrazine and the mixture was evaporated to dryness. The crude product was purified by silica gel chromatography increasing the MeOH content of DCM in a stepwise manner from 5% to 8% and then to 20%. The product, compound 10, obtained was still subjected to RP-HPLC purification (Hypersil ODS2, 21.2×250 mm, 5 μm) using a gradient elution from 25% aq. MeCN to 40% aq. MeCN. Yield 35%. ¹H NMR (CD₃OD+D₂O) mixture of R_{P} ans S_{P} diastereomers δ 8.72 and 8.74 (2×s, 1H, H5), 6.01 and 6.02 (2×d, J=3.0 Hz, 1H, H1′), 4.41-4.59 (m, 6H, H2′, H3′, CH₂OAc and CH₂OP), 4.14-4.31 (m, 7H, H4′, H5′, H5″, CH₂CH₃), 3.78 and 3.84 (2×dd, J=9.3 and 7.2 Hz, H^{α}-Ala), 3.72 and 3.74 (2×s, 3H, MeO-Ala), 2.07 and 2.08 (2×s, 3H, Ac), 1.32 and 1.34 (d, J=7.2 Hz, 3H, Me Ala), 1.24-1.28 (m, 6H, CH₂CH₃). ¹³C NMR (CD₃OD+D₂O) δ 174.7 (C═O Ala), 171.6 (COOEt), 167.0 (OCOMe), 161.9 (CONH₂), 157.0 (C3), 145.7 (C5), 92.1 (C1′), 83.0 (C4′), 74.6 (C2′), 70.2 (C3′), 66.4 (C5′), 61.3 (CH₂CH₃), 58.0 (spiro C), 52.0 (MeO-Ala), 50.0 (C^{α}-Ala), 19.5 (Ac), 19.0 (Me Ala), 13.0 (CH₃CH₃). ³¹P NMR (CD₃OD+D₂O)=8.1 and 8.0. HRMS: [M+H]⁺ obsd. 654.2022, calcd. 654.2018; [M+Na]⁺ obsd. 676.1876, calcd. 676.1838; [M+K]⁺ obsd. 692.1588, calcd. 692.1577.

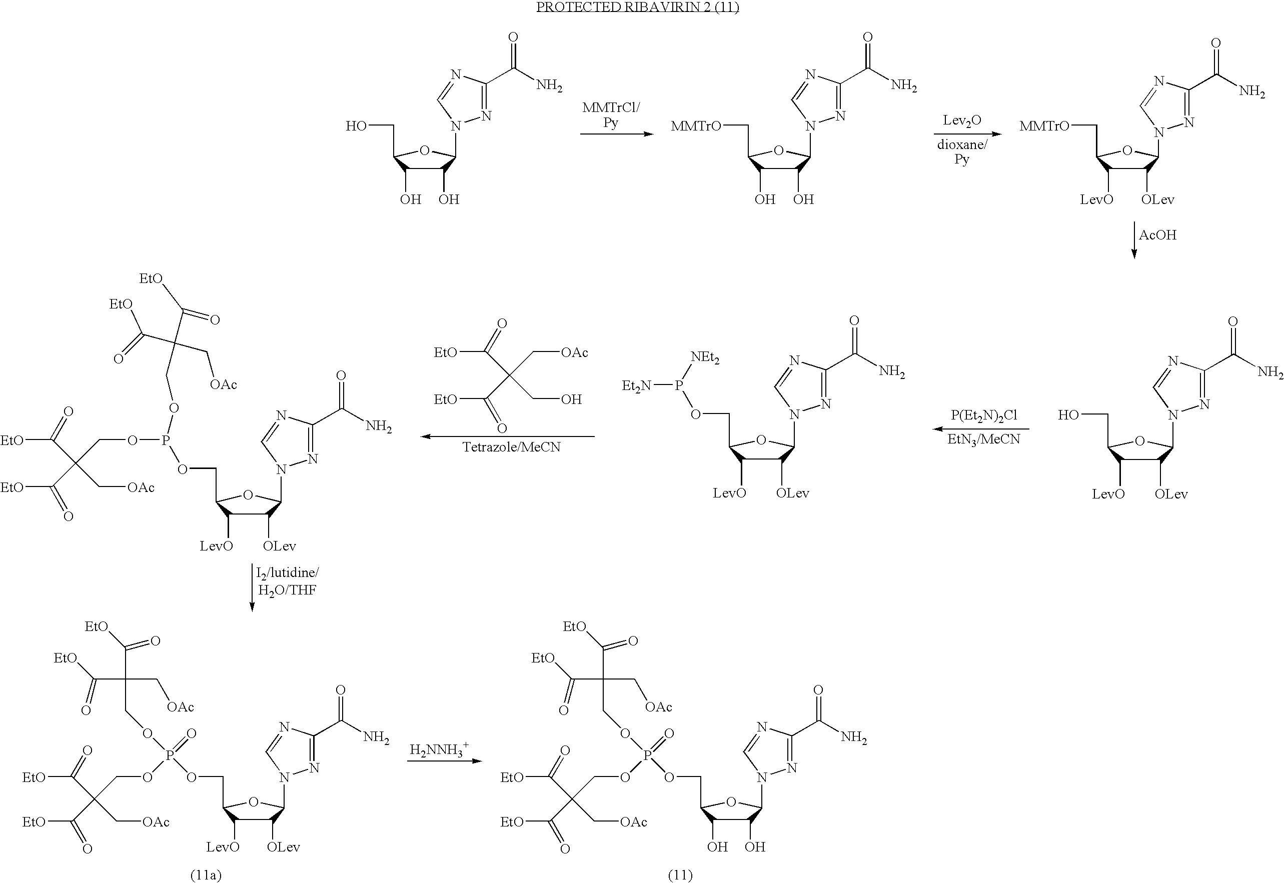

2′,3′-di-O-Levulinoylribavirin 5′-bis[3-acetyloxy-2,2-bis(ethoxycarbonyl)propyl]phosphate (11a). 2′,3′-di-O-Levulinoylribavirin (3.1 mmol; 1.38 g), coevaporated from dry MeCN and stored on P₂O₅ for 24 h, was dissolved in dry DCM (6.0 mL) under nitrogen and bis(diethylamino)chlorophosphine (4.4 mmol; 0.92 mL) was added. After 2 hours, the reaction mixture was passed through a short silica gel column (dried in oven) eluting with ethyl acetate containing 0.5% triethylamine. The elute was evaporated to dryness and the residue was coevaporated three times from MeCN to remove the traces of triethylamine. The product was dissolved in dry MeCN (2.0 mL) and diethyl 2-acetyloxymethyl-2-hydroxymethylmalonate (4.3 mmol; 1.126 g) dried on P₂O₅ was added. The solution was mixed with a solution of tetrazole (7.8 mmol) in MeCN (17.3 mL). The reaction was allowed to proceed for 1 h. Iodine (1.61 mmol; 0.41 g) in a mixture of THF (6.0 mL), H₂O (3.0 mL) and 2,6-lutidine (1.5 mL) was added and the mixture was stirred overnight. Aqueous NaHSO₃ (50 mL of 5% solution) was added and the product was extracted in DCM (2×40 mL and 2×30 mL). The organic phase was dried on Na₂SO₄ and evaporated to dryness. The product was purified by silica gel chromatography using 10-15% MeOH in DCM as an eluent. The yield was 4%. ¹H NMR (CDCl₃) δ 8.42 (s, 1H, H5), 7.55 (s, 1H, NH), 6.06 (d, J=3.6 Hz, 1H, H1′), 6.01 (s, 1H, NH), 5.65 (dd, J=3.6 and 5.3 Hz, 1H, H2′), 5.50 (dd, J=5.3 and 5.5, 1H, H3′), 4.40-455 (m, 9H, 2×CH₂OP, 2×CH₂OAc and H4′), 4.15-4.25 (m, 10H, 4×OCH₂Me, H5′ and H5″), 3.77 (dd, J=12.7 and 2.8 Hz, 1H, H5″), 2.73-2.78 (m, 4H, Lev), 2.59-2.65 (m, 4H, Lev), 2.17 (s, 3H, Ac), 2.15 (s, 3H, Ac), 2.02 (s, 3H, Lev), 2.01 (s, 3H, Lev), 1.22 (q, J=7.0 Hz, 12H, 4×OCH₂CH₃). ¹³C NMR (CDCl₃) δ 206.3 (C═O Lev), 206.2 (C═O Lev), 171.6 (C═O Lev), 171.5 (C═O Lev), 171.3 (2×Ac), 170.2 (4×COOEt), 166.3 (C═O), 157.0 (C3), 144.7 (C5), 90.1 (C1′), 81.4 (C4′), 74.5 (C3′), 70.4 (C2′), 66.8 (C5′), 65.3 (CH₂OP), 65.2 (CH₂OP), 62.3 (4×OCH₂CH₃), 61.2 (CH₂OAc), 61.1 (CH₂OAc), 57.9 (2×spiro C), 37.6 (Lev), 29.7 (Lev), 27.4 (Lev), 20.6 (2×Ac), 13.9 (4×OCH₂CH₃).

Ribavirin 5′-bis[3-acetyloxy-2,2-bis(ethoxycarbonyl)propyl]phosphate (11). Compound IIa (0.10 mmol; 0.10 g) was treated with hydrazinium acetate (0.55 mL of 0.5 mol L⁴ in a 4:1 mixture of pyridine and AcOH) for 45 min. The reaction was quenched with acetone (20 μL). The crude product was purified by RP-HPLC (Hypersil ODS; 10×250 mm; 5 μm) using isocratic elution with 40% MeCN in H₂O. The yield was 73%. ¹H NMR (CDCl₃) δ 8.51 (s, 1H, H5), 7.62 (s, 1H, NH), 6.38 (s, 1H, NH), 6.00 (d, J=2.4 Hz, 1H, H1′), 5.05 (br s, 1H, OH), 4.43-4.62 (m, 11H, 2×CH₂OP, 2×CH₂OAc, H2′, H3′ and H4′), 4.18-4.30 (m, 11H, 4×OCH₂Me, OH, H5′ and H5″), 2.06 (s, 3H, Ac), 2.04 (s, 3H, Ac), 1.22-1.29 (m, 12H, 4×OCH₂CH₃). ¹³C NMR (CDCl₃) 170.4 (2×Ac), 166.3 (4×COOEt), 161.3 (C═O), 157.0 (C3), 144.9 (C5), 92.6 (C1′), 82.9 (C4′), 75.2 (C2′), 70.4 (C3′), 67.7 (C5′), 65.4 (2×CH₂OP), 62.4 (4×OCH₂CH₃), 61.3 (2×CH₂OAc), 57.9 (2×spiro C), 20.6 (2×Ac), 13.9 (4×OCH₂CH₃). MS [M+H]⁺ obsd. 813.6, calcd. 813.2; [M+Na]⁺ obsd. 835.5, calcd. 835.2; [M+K]⁺ obsd. 851.5, calcd. 851.2.

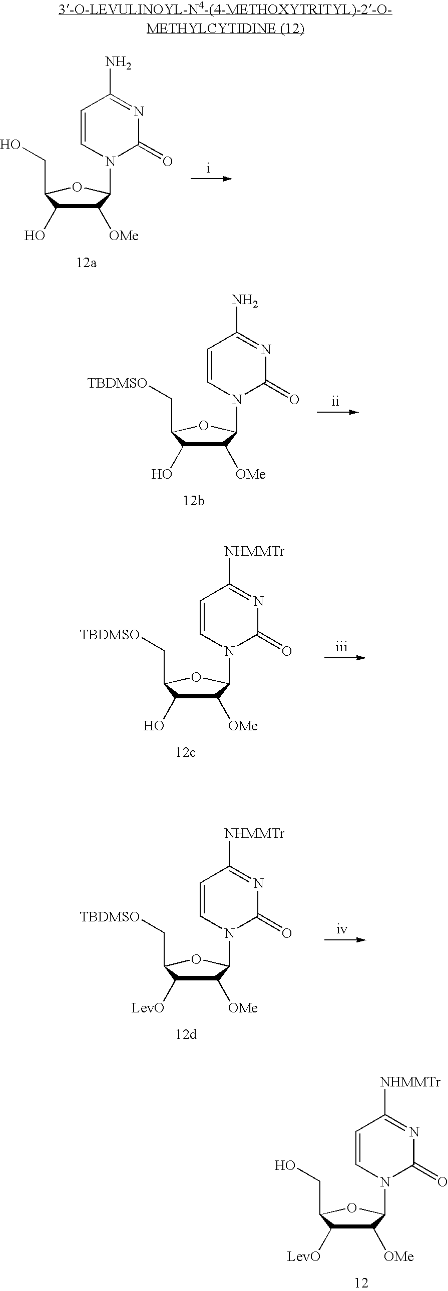

5′-O-(tert-Butyldimethylsilyl)-2′-O-methylcytidine (12b). 2′-β-methylcytidine (12a; 18.4 mmol; 4.74 g) was coevaporated twice from dry pyridine, dried over P₂O₅ (24 h) and dissolved in dry pyridine (20 mL). tert-Butyldimethylsilyl chloride (TBDMSCl; 20.2 mmol; 3.05 g) was added and the mixture was agitated at room temperature overnight. The unreacted TBDMSCl was quenched with MeOH, the mixture was evaporated to dryness and the residue was subjected to chloroform/aq. NaHCO₃ work-up. The yield of the crude product dried on Na₂SO₄ was nearly quantitative. It was used for 4-methoxytritylation of the amino group without further purification. ¹H NMR (CDCl₃): δ 8.14 (d, J=7.5 Hz, 1H, H6), 6.00 (d, J=1.1 Hz; 1H, H1′), 6.82 (d, J=7.5 Hz, 1H, H5), 4.22 (dd, J=8.0 and 5.1 Hz, 1H, H3′), 4.09 (dd, J=11.8 and 1.8 Hz, 1H, H5′), 3.97 (m, 1H, H4′), 3.87 (dd, J=11.8 and 1.6, 1H, H5″), 3.73 (dd, J=5.1 and 1.0 Hz, 1H, H2′), 3.67 (s, 3H, 2′-OMe), 0.94 (s, 9H, Me₃C—Si), 0.13 (s, 3H, Me-Si), 0.13 (s, 3H, Me-Si).

5′-O-(tert-Butyldimethylsilyl)-N⁴-(4-methoxytrityl)-2′-O-methylcytidine (12c). Compound 12b (18.4 mmol; 6.84 g) was coevaporated twice from dry pyridine and dissolved in the same solvent (20 mL). 4-Methoxytrityl chloride (18.4 mmol; 5.69 g) was added and the mixture was agitated at 45° C. for 24 h. MeOH (20 mL) was added, the mixture was evaporated to dryness and the residue was subjected to chloroform/aq. NaHCO₃ work-up. Silica gel chromatography with DCM containing 2-5% MeOH gave compound 8c as a solid foam in 46% overall yield starting from 2′-O-methylcytidine. ¹H NMR (CDCl₃) δ 7.91 (d, J=7.7 Hz, 1H, H6), 7.26-7.33 (m, 6H, MMTr), 7.21-7.23 (m, 4H, MMTr), 7.13-7.15 (m, 2H, MMTr), 6.82-6.85 (m, 2H, MMTr), 6.77 (br. s, 1H, NH), 5.99 (s, 1H, H1′), 5.00 (d, J=7.7 Hz, 1H, H5), 4.12 (m, 1H, H3′), 4.02 (dd, J=11.9 and 1.2 Hz, 1H, H5′), 3.86-3.88 (m, 1H, H4′), 3.81 (dd, J=11.9 and 1.2 Hz, 1H, H5″), 3.81 (s, 3H, MeO-MMTr), 3.72-3.74 (m, 4H, H2′ and 2′-OMe), 2.63 (br s, 1H, 3′-OH), 0.75 (s, 9H, Me₃C—Si), −0.03 (s, 3H, Me-Si), −0.05 (s, 3H, Me-Si). ¹³C NMR (CDCl₃) δ 165.6 (C4), 158.7 (MMTr), 155.1 (C2), 144.4 (MMTr), 144.3 (MMTr), 140.9 (C6), 136.0 (MMTr), 130.0 (MMTr), 128.6 (MMTr), 128.3 (MMTr), 127.5 (MMTr), 113.6 (MMTr), 94.2 (C5), 87.6 (C1′), 83.9 (C2′), 83.7 (C4′), 70.5 (MMTr), 66.8 (C3′), 60.5 (C5′), 58.8 (2′-OMe), 55.2 (MMTr), 25.8 (TBDMS), 18.3 (TBDMS), −5.6 (TBDMS), −5.7 (TBDMS).

5′-O-(tert-Butyldimethylsilyl)-3′-O-levulinoyl-N⁴-(4-methoxytrityl)-2′-O-methylcytidine (12d). Levulinic acid (21.6 mmol; 2.51 g) was dissolved in dry dioxane and dicyclohexylcarbodiimide (11.1 mmol; 2.28 g) was added portionwise during 1 h at 0° C. The mixture was allowed to warm up to reduce its viscosity and it was then filtrated to a solution of compound 12c (8.46 mmol; 5.45 g) in pyridine (18 mL). The mixture was agitated overnight, evaporated to dryness and the residue was subjected to DCM/NaHCO₃ work-up. The organic phase was dried on Na₂SO₄, evaporated to dryness and the residue was purified by Silica gel chromatography using DCM containing 1% MeOH as an eluent. Yield 86%. ¹H NMR (CDCl₃) δ 7.81 (d, J=7.7 Hz, 1H, H6), 7.27-7.34 (m, 6H, MMTr), 7.22-7.23 (m, 4, MMTr), 7.14-7.15 (m, 2H, MMTr), 6.84-6.86 (m, 2H, MMTr), 6.80 (br. s, 1H, NH), 6.07 (d, J=1.5 Hz, 1H, H1′), 4.99 (d, J=7.7 Hz, 1H, H5), 4.97 (dd, J=7.9 and 5.0 Hz, 1H, H3′), 4.21 (m, 1H, H2′), 3.99-4.01 (m, 2H, H4′ and H5′), 3.81 (s, 3H, MeO-MMTr), 3.70 (dd, J=12.0 and 1.3 Hz, 1H, H5″), 3.57 (s, 3H, 2′-OMe), 2.63-2.83 (m, 4H, Lev), 2.21 (s, 3H, Lev), 0.74 (s, 9H, Me₃C—Si), −0.05 (s, 3H, Me-Si), −0.07 (s, 3H, Me-Si). ¹³C NMR (CDCl₃) δ 206.1 (Lev), 172.0 (Lev), 165.5 (C4), 158.7 (MMTr), 155.1 (C2), 144.4 (MMTr), 144.3 (MMTr), 140.7 (C6), 136.0 (MMTr), 130.0 (MMTr), 128.6 (MMTr), 128.3 (MMTr), 127.5 (MMTr), 113.6 (MMTr), 94.4 (C5), 88.4 (C1′), 82.5 (C2′), 81.3 (C4′), 70.6 (MMTr), 69.1 (C3′), 60.8 (C5′), 58.9 (2′-OMe), 55.2 (MMTr), 37.8 (Lev), 29.8 (Lev), 27.8 (Lev), 25.7 (TBDMS), 18.2 (TBDMS), −5.7 (TBDMS), −5.8 (TBDMS).

3′-O-Levulinoyl-N⁴-(4-methoxytrityl)-2′-O-methylcytidine (12). Compound 12d (3.40 mmol; 2.52 g) was dissolved into a mixture THF (48 mL) and AcOH (9 mL) containing tetrabutylammonium fluoride (6.85 mmol; 1.79 g). The mixture was agitated for 2 days and then evaporated to dryness. The residue was dissolved into EtOAc (50 mL), washed with water, aq. NaHCO₃ and brine, and dried on Na₂SO₄. The compound 12 was obtained as a white foam in virtually quantitative yield. ¹H NMR (CDCl₃) δ 7.22-7.34 (m, 11H, H6 and MMTr), 7.12-7.15 (m, 2H, MMTr), 6.89 (br. s, 1H, NH), 6.83-6.85 (m, 2H, MMTr), 5.41 (d, J=5.0 Hz, 1H, H1′), 5.31 (dd, J=4.6 and 4.7, 1H, H4′), 5.07 (d, J=7.6 Hz, 1H, H5), 4.58 (dd, J=5.0 and 5.0 Hz, 1H, H3′), 4.18 (m, 1H, H2′), 3.90 (d, J=12.7 Hz, 1H, H5′), 3.81 (s, 3H, MeO-MMTr), 3.71 (dd, J=12.7 and 4.7 Hz, 1H, H5″), 3.45 (s, 3H, 2′-OMe), 2.75-2.80 (m, 2H, Lev), 2.63-2.66 (m, 2H, lev), 2.20 (s, 3H, Lev).

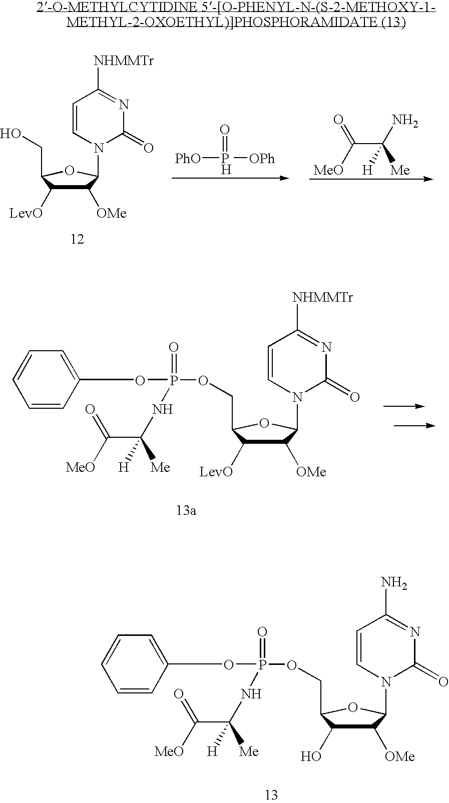

3′-O-Levulinoyl-N⁴-(4-methoxytrityl)-2′-O-methylcytidine 5′-[O-phenyl-N-(S-2-methoxy-1-methyl-2-oxoethyl)]phosphoramidate (13a). Compound 12 (2.58 mmol; 1.62 g) dried on P₂O₅ for 2 days was dissolved in dry pyridine (5 mL) and diphenylphosphite (3.09 mmol; 595 μL) was added under nitrogen. After half an hour, carefully dried L-alanine methyl ester (3.94 mmol; 0.55 g) in a mixture of dry pyridine (1 mL) and MeCN (6 mL) was added. CCl₄ (15 mL) and triethylamine (18.1 mmol; 2.54 mL) was added and the reaction was allowed to proceed for 70 min. Volatiles were removed under reduced pressure and the residue was purified by silica gel chromatography increasing the MeOH content of DCM from 1 to 10% in a stepwise manner. Compound 13a was obtained as a white foam in 70% yield. ¹H NMR (CDCl₃) mixture of R_{P} and S_{P} diastereomers δ 7.02-7.35 (m, 17H, MMTr and Ph), 6.80-6.85 (m, 3H, MMTr and N⁴H), 5.99 and 6.02 (2×d, J=3.2 Hz, 1H, H1′), 4.90-5.00 (m, 2H, H3′ and H4′), 3.88-4.43 (m, 4H, H5, H2′, H5′, H5″), 3.80 (s, 3H, MMTr), 3.68-3.75 (m, 1H, H^{α}-Ala, 3.63 and 3.64 (2×s, 3H, MeO-Ala), 3.46 and 3.52 (2×s, 3H, 2′-OMe), 2.74-2.81 (m, 2H, Lev), 2.59-2.64 (m, 2H, Lev), 2.19 and 2.20 (2×s, 3H, Lev), 1.88 (br s, 1H, NH—P), 1.27 and 1.31 (2×d, J=7.1 Hz, Me Ala).

2′-O-Methylcytidine 5′-[O-phenyl-N-(S-2-methoxy-1-methyl-2-oxoethyl)]phosphoramidate (13). Compound 13a (1.81 mmol; 1.57 g) was dissolved in a mixture of hydrazine hydrate (7.2 mmol; 350 μL), pyridine (11.5 mL) and AcOH (2.88 mL) and the reaction was allowed to proceed for 5 h. Volatiles were removed under reduced pressure and the residue was dissolved in DCM (50 mL) and washed with water, aq. NaHCO₃ and brine. The organic phase was dried on Na₂SO₄, evaporated to dryness and the residue was purified by silica gel chromatography using DCM containing 4-6% MeOH as an eluent.

The purified product was dissolved 80% aq. AcOH (8 mL) and the mixture was allowed to proceed at 55° C. for 2 h and additionally at 65° C. for 4.5 h. The mixture was evaporated to dryness and the residue was coevaporated twice from water and then purified by silica gel chromatography using gradient elution from 7 to 20% MeOH in DCM. The overall yield from 13 was 50%. ¹H NMR (CDCl₃) mixture of two diastereomers δ 7.64 and 7.68 (2×d, J=7.4, 1H, H6), 7.26-7.33 (m, 2H, Ph), 7.20-7.24 (m, 2H, Ph), 7.13-7.16 (m, 1H, Ph), 6.32 (br s, 2H, NH₂), 5.90 and 5.94 (2×s, 1H, H1′), 5.69 and 5.82 (2×d, J=7.4, 1H, H5), 4.35-4.55 (m, 2H, H5′ and H5″), 4.12-4.18 (m, 2H, H3′ and H4′), 3.98-4.08 (m, 2H, α-H-Ala and 3′-OH), 3.72-3.76 (m, 1H, 2′-OMe), 3.67 and 3.68 (2×s, 3H, MeO-Ala), 3.58 and 3.60 (2×s, 3H, 2′-OMe), 2.45 (br s, 1H, NH—P), 1.37 and 1.39 (2×d, J=7.2 Hz, 3H, Me-Ala). ¹³C NMR (CDCl₃) δ174.2 (C═O Ala), 166.0 (C4), 155.9 (C2), 150.5 (Ph), 140.6 (C6), 129.8 (Ph), 125.1 (Ph), 120 (Ph), 95.1 (C5), 88.4 (C1′), 83.4 (C2′), 81.4 (C4′), 68.1 (C3′), 65.1 (C5′), 58.6 (2′-OMe), 52.5 (MeO-Ala), 50.3 (C^{α}-Ala), 20.7 (Me-Ala). ³¹P NMR δ 3.1 and 3.3. HRMS [M+H]⁺ obsd. 499.1590, calcd. 499.1583; [M+Na]⁺ obsd. 521.1438, calcd. 521.1408, [M+K]⁺ obsd. 537.1149, 537.1147.

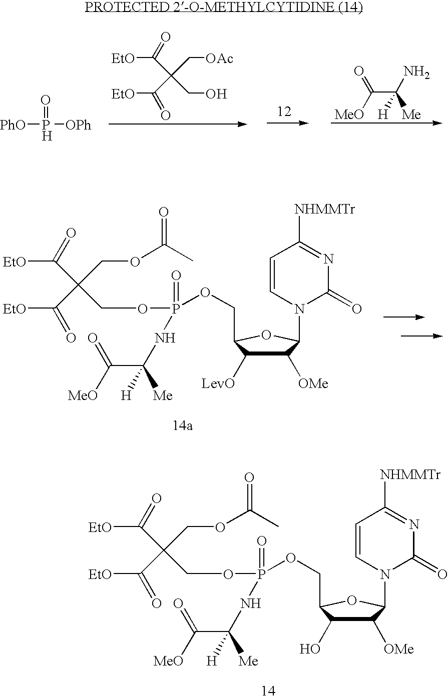

3′-O-Levulinoyl-N⁴-(4-methoxytrityl)-2′-O-methylcytidine 5′-[O-3-acetyloxy-2,2-bis(ethoxycarbonyl)propyl-N-(S-2-methoxy-1-methyl-2-oxoethyl)]phosphoramidate (14a). Diphenylphosphite (2.83 mmol; 545 μL) was dissolved in dry pyridine (2.0 mL) and diethyl 2-acetyloxymethyl-2-hydroxymethylmalonate (2.36 mmol; 0.62 g) in dry pyridine (2.0 mL) was dropwise added under nitrogen. After 40 min from the beginning of the reaction, compound 12 (3.30 mmol; 2.07 g) in dry pyridine (4 mL) was added dropwise under nitrogen. After 2.5 hours, methyl ester of L-alanine (2.85 mmol; 0.398 g) in dry pyridine (1 mL) was added. Finally, dry MeCN (9.0 mL), CCl₄ (14.0 mL) and distilled triethylamine (16.5 mmol; 2.33 mL) were added to the mixture and the reaction was allowed to proceed for 1 h. Volatiles were removed under reduced pressure and the residue was dissolved in DCM (50 mL) and washed with water, aq. NaHCO₃ and brine. The organic phase was dried on Na₂SO₄ and concentrated to yellow oil. Purification by silica gel chromatography using DCM containing 2-3% MeOH as an eluent, gave compound 14a in 24% yield. ¹H NMR (CDCl₃), mixture of S_{P} and R_{P} diastereomers δ 7.21-7.33 (m, 11H, H6 and MMTr), 7.13-7.15 (m, 2H, MMTr), 6.89 (br. s, 1H, NH), 6.82-6.84 (m, 2H, MMTr), 5.96 and 5.97 (2×d, J=2.5 Hz, 1H, H1′), 5.41 and 5.30 (2×d, J=5.0 Hz, 1H, H4′), 5.13 and 5.14 (2×d, J=5.0, 1H, H3′), 5.06 (d, J=7.6 Hz, 1H, H5), 4.84-4.93 (m, 1H, H2′), 4.57-4.60 (m, 2H, CH₂OAc), 4.48-4.52 (m, 2H, CH₂OP), 4.37-4.48 (m, 2H, H5′ and H5″), 4.10-4.25 (m, 6H, OCH₂Me), 3.98 (m, 1H, α-H-Ala), 3.80 (s, 3H, MeO-MMTr), 3.63 and 3.64 (2×s, 3H, MeO-Ala), 3.49 (s, 3H, 2′-OMe), 2.73-2.78 (m, 2H, Lev), 2.61-2.65 (m, 2H, Lev), 2.19 (s, 3H, Lev), 2.00 and 2.01 (2×s, 3H, OAc), 1.77 (br s, NH-Ala), 1.24-1.26 (m, 9H, Me-Ala and —CH₂CH₃).

2′-O-Methylcytidine 5′-[O-3-acetyloxy-2,2-bis(ethoxycarbonyl)propyl-N-(S-2-methoxy-1-methyl-2-oxoethyl)]phosphoramidate (14). Compound 14a (0.73 mmol; 0.760 g) was dissolved into a mixture of hydrazine hydrate (1.59 mmol; 50 μL), pyridine (1.6 mL) and AcOH (0.4 mL). The reaction was allowed to proceed for 75 min. Unreacted hydrazinium acetate was then quenched with acetone and volatiles were removed under reduced pressure and the residue was purified by silica gel chromatography using a 1:10 mixture (v/v) of MeOH and EtOAc as eluent). The residue (410 mg) obtained by evaporation to dryness was dissolved in 80% aq. AcOH and the reaction was allowed to proceed overnight. The product was purified by RP-HPLC on a SunFire prep C18 column (10×250 mm, 5 μm) using a stepwise gradient elution from 20% MeCN in water to 40% MeCN in H₂O. The yield was 10%. ¹H NMR (CDCl₃), mixture of S_{P} and R_{P} diastereomers δ 7.71 and 7.74 (2×d, J=7.5 Hz, 1H, H6), 5.90-5.94 (m, 2H, H1′ and H5), 4.57-4.66 (m, 2H, CH₂OAc), 4.41-4.52 (m, 2H, CH₂OP), 4.35-4.39 (m, 1H, H5′), 4.21-4.28 (m, 5H, H5″ and OCH₂Me), 4.13-4.16 (m, 1H, H3′), 4.09-4.10 (m, 1H, H4′), 3.92-3.94 (m, 1H, α-H-Ala), 3.80-3.82 (m, 1H, H2′), 3.73 and 3.75 (2×s, 3H, MeO-Ala), 3.67 (s, 3H, 2′-OMe), 2.06 and 2.07 (2×s, 3H, OAc), 1.41 and 1.42 (d, J=7.1 Hz, 3H, Me-Ala), 1.24-1.29 (m, 6H, —CH₂CH₃). ¹³C NMR (CDCl₃) δ 174.3 (C═O Ala), 174.2 (C═O Ac), 170.5 (COOEt), 166.1 (C4), 166.0 (C2), 140.6 (C6), 94.7 (C5), 88.5 (C1′), 83.4 (C2′), 81.4 (C4′), 68.0 (C3′), 64.2 (C5′), 64.0 (CH₂OP), 62.3 (CH₂Me), 61.5 (CH₂OAc), 58.6 (MeO-2′), 58.1 (spiro C), 52.5 (MeO-Ala), 50.0 (C^{α}-Ala), 20.8 (Ac), 20.7 (Me-Ala), 14.0 (CH₂CH₃). ³¹P NMR (CDCl₃) 7.5 and 7.7. HRMS: [M+H]⁺ obsd. 667.2212, calcd. 667.2222; [M+Na]⁺ obsd. 689.2023, calcd. 689.2042; [M+K]⁺ obsd. 705.1747, calcd. 707.1781.

### 3′-O-levulinoylthymidine

5′-O-(4,4-dimethoxytrityl)thymidine (16.2 mmol, 8.8 g) prepared by established synthetic procedures, was dissolved in anhydrous 1,4-dioxane (100 mL). A solution of levulinic anhydride, prepared from levulinic acid (49.0 mmol, 5.70 g) in pyridine (60 mL) using 1,3-dicyclohexylcarbodiimide (48.4 mmol, 10.0 g) as a condensing agent, was filtered onto the nucleoside. After stirring for 4 h at RT, the reaction mixture was evaporated to dryness. The dimethoxytrityl group was removed with 80% aq. acetic acid (80 mL). The reaction mixture was evaporated to dryness and the residue was purified on silica gel column eluted with a mixture of DCM and MeOH (90:10, v/v). The product was obtained as a white power in 50% yield (2.8 g). ¹H NMR (500 MHz, CDCl₃): δ-9.36 (s, 1H, NH), 7.73 (s, 1H, H6), 6.25 (dd, 1H, J=6.5 and 2.0 Hz, H1′), 5.37 (m, 1H, H4′), 4.11 (d, 1H, J=2.0 Hz, H3′), 3.90 (m, 2H, H5′ and H5″), 2.80 (2H, CH₂ of levulinyl), 2.59 (t, 2H, J=6.0 Hz, CH₂ of levulinyl), 2.42 (m, 2H, H2′ and H2″), 2.22 (s, 3H, CH₃ of levulinyl), 1.92 (s, 3H, CH₃). ESI⁺-MS: m/z obsd. 341.6 [M+H]⁺, calcd. 341.1.

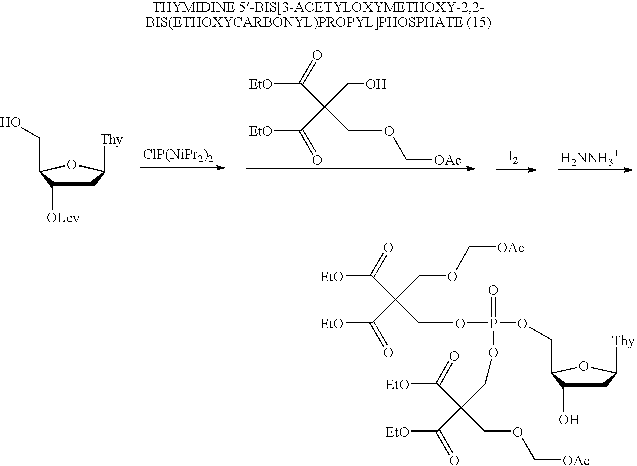

3′-O-Levulinoylthymidine (0.47 mmol; 0.166 g) was coevaporated once from dry pyridine and three times from dry MeCN and dissolved in dry DCM (1.2 mL) under nitrogen. Triethylamine (2.35 mmol; 0.34 mL) and bis(diethylamino)chlorophosphine (0.68 mmol; 0.145 mL) were added and the mixture was stirred under nitrogen for 2 h. The product was isolated by passing the mixture through a short silica gel column with a 4:1 mixture of ethyl acetate and hexane containing 0.5% triethylamine. The solvent was removed under reduced pressure and the residue was coevaporated three times from dry MeCN to remove the traces of triethylamine. The residue was dissolved in dry MeCN (1.0 mL) and 3-acetyloxymethoxy-2,2-bis(ethoxycarbonyl)propanol (1.68 mmol; 0.49 g) in dry MeCN (1.0 mL) and tetrazole (2.91 mmol; 6.46 mL of 0.45 mol L⁻¹ solution in MeCN) were added under nitrogen. The reaction was allowed to proceed for 6 h and then iodine (0.73 mmol; 0.185 g) in a mixture of THF (4.0 mL), H₂O (2.0 mL) and 2,6-lutidine (1.0 mL) was added. The oxidation was allowed to proceed overnight. The excess of iodine was destroyed with 5% NaHSO₃. The mixture was extracted three times with DCM. The organic phase was washed with brine, dried on Na₂SO₄ and evaporated to dryness. The crude product was purified on a silica gel column eluting with DCM containing 5-10% MeOH. The yield was 15%.

3′-O-Levulinoylthymidine 5′-bis[3-acetyloxymethoxy-2,2-bis(ethoxycarbonyl)propyl]phosphate (0.071 mmol; 69 mg) was dissolved in dry DCM (2.0 mL) and hydrazine acetate (0.12 mmol; 11 mg) in dry MeOH (0.20 mL) was added. After 1 h, hydrazinium acetate (0.05 mmol; 4.6 mg) in a mixture of DCM (100 μL) and MeOH (20 μL) was added. The reaction was allowed to proceed for 2 h and the addition of hydrazinium acetate was repeated. The reaction was quenched with acetone and the mixture was evaporated to dryness. The product was purified on a silica gel column eluting first with ethyl acetate and then with DCM containing 15% MeOH. The yield was quantitative. ¹H NMR (CDCl₃) δ 8.91 (s, 1H, N3H), 7.34 (s, 1H, H6), 6.31 (dd, J=6.0 and 6.0 Hz, 1H, H1′), 5.25 (s, 4H, OCH₂O), 4.54 (m, 5H, 2×CH₂OCH₂OAc and H3′), 4.24 (m, 10H, 4×OCH₂Me, H5′ and H5″), 4.05 (s, 1H, H4′), 3.61 (br s, 1H, 3′-OH), 2.42 (m, 1H, H2′), 2.24 (m, 1H, H2″), 2.11 (s, 6H, 2×Ac), 1.95 (s, 3H, 5-Me), 1.27 (m, 12H, 4×OCH₂CH₃). ¹³C NMR (CDCl₃) δ 170.6 (Ac), 166.6 (COOEt), 163.7 (C4=0), 150.3 (C2=0), 135.5 (C6), 111.4 (C5), 88.8 (OCH₂O), 84.8 (C4′), 84.4 (C1′), 70.7 (C3′), 67.2 (POCH₂), 67.0 (C5′), 65.3 (CH₂OCH₂Oac), 62.3 (OCH₂Me), 58.8 (spiro C), 39.6 (C2′), 20.9 (Ac), 13.9 (OCH₂CH₃), 12.4 (5-Me). ³¹P NMR (acetone) δ −2.1 ppm. MS [M+Na]⁺ obsd. 893.8, calcd. 893.3.

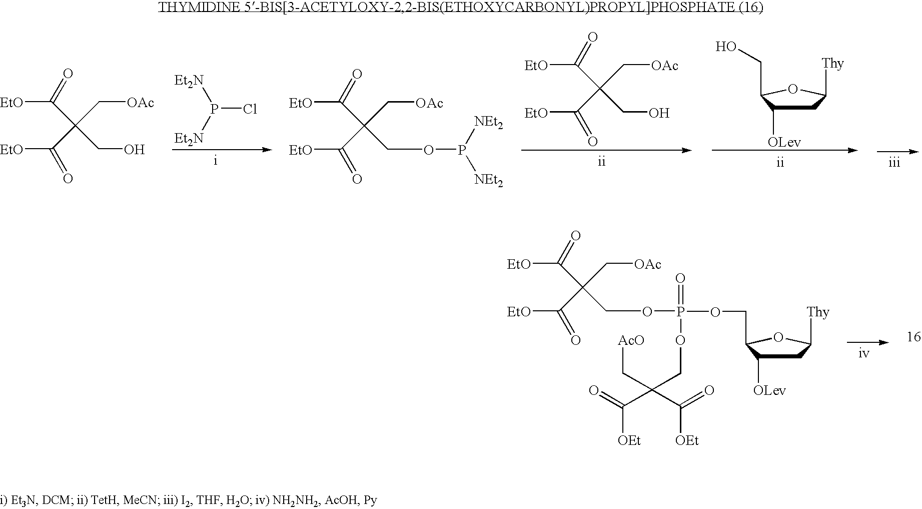

The hydroxy precursor was coevaporated once from dry pyridine and three times from dry MeCH after which it was dried over P₂O₅ overnight. To a solution of the dried hydroxy precursor (2.9 mmol, 0.76 g) in dry DCM (2 mL), anhydrous triethylamine (14.4 mmol, 2 mL) and bis(diethylamino)chlorophosphine (4.0 mmol; 850 μL) was added, and the reaction mixtures was stirred for 1 h under nitrogen. The product was filtered through a short silica gel column eluting with a mixtures of anhydrous ethyl acetate and triethylamine in hexane (60:0.5:39.5, v/v/v). The solvent was removed under reduced pressure and the residue was coevaporated three times from dry MeCN to remove the traces of triethylamine. The residue was dissolved in dry MeCN (2.0 mL) and the hydroxy precursor (2.9 mmol, 0.77 g) in dry MeCN (2.0 mL) and tetrazole (7.2 mmol, 16.0 mL of 0.45 mol L⁻¹ solution in MeCN) was added under nitrogen. The reaction mixture was stirred for 2.5 h at RT. 3′-O-levulinoylthymidine (2.9 mmol, 0.1 g) dried over phosphorus pentoxide, and 1-H-tetrazole (2.9 mmol, 6.4 mL of 0.45 mol L⁻¹ solution in MeCN) was added and the stiffing was continued for 1.5 h. The phosphite ester formed was oxidized with I₂ (0.1 mol L⁻¹) in a mixture of THF, H₂O and lutidine (4:2:1, v/v/v, 10 mL). The crude product was isolated by DCM/aq. NaHSO₃ work up, and purified on a silica gel column eluated with a mixture of hexane and ethyl acetate (40:60 v/v) as an eluent. ¹H NMR (500 MHz, CDCl₃): δ=8.75 (s, 1H, NH), 7.48 (d, J=1.0 Hz, H6), 6.37 (dd, J=7.0 and 5.5 Hz, H1′), 5.25 (d, J=6.5 Hz, H4′), 4.65-4.48 (m, 19H, CH₂, CH₃CH₂, H5′, H5″ and H3′), 2.78 (t, 2H, CH₂ of levulinoyl), 2.60 (t, 2H, CH₂ of levulinoyl), 2.42 (dd, 1H, H2′), 2.22 (dd, 1H, H2″), 2.21 (s, 3H, CH₃ of levulinoyl), 2.063 (s, 3H, Ac), 2.056 (s, 3H, Ac), 1.97 (d, 3H, CH₃) 1.27 (m, 12H, 4×CH₃CH₂). ESI⁺-MS: m/z obsd. 909.8 [M+H]⁺, calcd. 909.8.

A mixture of NH₂NH₂.H₂O (0.5 mol L⁻¹), the product from the previous step (0.14 mmol, 0.12 g), pyridine (0.9 mL) and acetic acid (0.2 mL) was stirred for 80 min at 0° C. The ice-water bath was removed and the solution was stirred for additional 11 h at RT. The crude 16 was isolated by DCM/aq. NaHCO₃ work up and purified on a silica gel column eluted with a mixture of DCM and MeOH (90:10, v/v) and by reverse phase chromatography on a Lobar RP-18 column (37×440 mm, 40-63 μm), eluting with a mixture of water and acetonitrile (60:40, v/v). The product, 16, was obtained as a clear oil in 55% yield. ³¹P NMR (202 MHz, D₂O): δ=−1.96 ppm. ¹H NMR (500 MHz, CDCl₃): δ=9.30 (s, 1H, NH), 7.35 (s, 1H, H6), 6.33 (dd, J=6.5 Hz, 1H, H1′), 4.64-4.48 (m, 9H, CH₂, H4′), 4.25 (m, 10H, CH₃CH₂, H5′ and H5″), 4.40 (br. s, 1H, H3′), 2.40 (m, 1H, H2′), 2.20 (dd, 1H, H2″), 2.06 (s, 6H, 2×Ac), 1.94 (s, 3H, CH₃), 1.25 (t, 12H, CH₃CH₂). ¹³C NMR (500 MHz, CDCl₃): δ=170.3 (Ac), 166.3 (COOEt), 163.9 (C4=0), 150.5 (C2=0), 135.5 (C6), 111.4 (C5), 84.7 (C4′), 84.3 (C1′), 84.2, 70.5 (C3′), 67.1 (POCH₂), 65.3 (CH₂OAc), 62.4 (OCH₂Me), 58.8 (spiro C), 39.7 (C2′), 20.6 (Ac), 13.9 (OCH₂CH₃), 12.4 (5-Me). ESI⁺-MS: m/z [M+H]⁺ obsd. 811.2517, calcd. 811.2460.

### Antiviral Activity of Selected Compounds

### HCV Replicon Assay

Antiviral activity of the test compounds was assessed (Okuse, et al., Antivir. Res. (2005) 65:23) in the stably HCV RNA-replicating cell line, AVA5 (genotype 1b, subgenomic replicon, Blight, et al., Sci. (2000) 290:1972). Compounds were added to dividing cultures daily for three days. Cultures generally start the assay at 30-50% confluence and reach confluence during the last day of treatment. Intracellular HCV RNA levels and cytotoxicity were assessed 72 hours after treatment.

Quadruplicate cultures for HCV RNA levels and cytoxicity (on 96-well plates) were used. A total of 12 untreated control cultures, and triplicate cultures treated with α-interferon (concentrations of: 10 IU/mL, 3.3 IU/mL, 1.1 IU/mL and 0.37 IU/mL) and 2′C-Me-C (concentrations of: 30 μM, 10 μM, 3.3 μM and 1.1 μM) served as assay controls.

Intracellular HCV RNA levels were measured using a conventional blot hybridization method, in which HCV RNA levels are normalized to the levels of β-actin RNA in each individual culture (Okuse, et al., Antivir. Res. (2005) 65:23). Cytotoxicity was measured using an established neutral red dye uptake assay (Korba and Gerin, Antivir. Res. (1992) 19:55; Okuse, et al., Antivir. Res. (2005) 65:23). HCV RNA levels in the treated cultures are expressed as a percentage of the mean levels of RNA detected in untreated cultures. The absorbance of the internalized dye at 510 nM (A₅₁₀) was used for quantitative analysis.

Compounds were dissolved in 100% tissue culture grade DMSO (Sigma, Inc.) at 10 mM. Aliquots of test compounds sufficient for one daily treatment were made in individual tubes and all material was stored at −20° C. For the test, the compounds were suspended into culture medium at room temperature, and immediately added to the cell cultures. Compounds were analyzed separately in two groups with separate assay controls. The concentrations of the test compounds were run at concentrations of 10 μM, 3.3 μM, 1.1 μM and 0.37 μM.

Values presented (±standard deviations [S.D.]) were calculated by linear regression analysis using data combined from all treated cultures. S.D. was calculated using the standard error of regression generated from the linear regression analyses (QuattroPro™). EC₅₀ and EC₉₀, drug concentrations at which a 2-fold, or a 10-fold depression of HCV RNA (relative to the average levels in untreated cultures), respectively, were observed; CC₅₀, drug concentrations at which a 2-fold depression of neutral red dye uptake (relative to the average levels in untreated cultures) were observed.

As shown by the results in Table 1, compound 9 was inactive. By comparison, compounds 10 and 11 showed activity. These results demonstrate the ability of the 2,2-disubstituted-acyl(oxyalkyl) group and the amino acid to neutralize the charge on the phosphate for entry into the cell. Additionally, the results show both groups have the ability to be removed once inside the cell.

TABLE 1

| Compound | CC₅₀ (μM) | EC₅₀ (μM) | EC₉₀ (μM) |
|---|---|---|---|
| 11 | 9.7 ± 0.2 | 1.6 ± 0.2 | 4.4 ± 0.4 |
| 10 | >100 | 8.7 | 54 |
| 9 | >100 | >100 | >100 |
| 12a | >100 | 1.6 | 5.9 |
| 13 | >100 | >100 | >100 |

### Kinetic Studies

Preparation of the cell extract. 10×10⁶ of human prostate carcinoma cells (PC3) are treated with 10 mL of RIPA-buffer [15 mM Tris-HCl pH 7.5, 120 mM NaCl, 25 mM KCl, 2 mM EDTA, 2 mM EGTA, 0.1% Deoxycholic acid, 0.5% Triton X-100, 0.5% PMSF supplemented with Complete Protease Inhibitor Cocktail (Roche Diagnostics GmBH, Germany)] at 0° C. for 10 min. Most of the cells are disrupted by this hypotonic treatment and the remaining ones are disrupted mechanically. The cell extract obtained is centrifuged (900 rpm, 10 min) and the pellet is discarded. The extract is stored at −20° C.

Stability of protected nucleotide analogs in the cell extract. The cell extract is prepared as described above (1 mL), and is diluted with a 9-fold volume of HEPES buffer (0.02 mol L⁻¹, pH 7.5, I=0.1 mol L⁻¹ with NaCl). A protected nucleotide analog (0.1 mg) is added into 3 mL of this HEPES buffered cell extract and the mixture is kept at 22±1° C. Aliquots of 150 μL are withdrawn at appropriate intervals, filtered with SPARTAN 13A (0.2 μm) and cooled in an ice bath. The aliquots are analyzed immediately by HPLC-ESI mass spectroscopy (Hypersil RP 18, 4.6×20 cm, 5 μm). For the first 10 min, 0.1% aq. formic acid containing 4% MeCN is used for elution and then the MeCN content is increased to 50% by a linear gradient during 40 min.

Stability of protected nucleotide analogs towards Porcine Liver Esterase. A protected nucleotide analog (1 mg) and 3 mg (48 units) of Sigma Porcine Liver Esterase (66H7075) are dissolved in 3 mL of HEPES buffer (0.02 mol L⁻¹, pH 7.5, I=0.1 mol L⁻¹ with NaCl). The stability test is carried out as described above for the cell extract.

Stability tests in human serum. Stability tests in human serum are carried out as described for the whole cell extract. The measurements are carried out in serum diluted 1:1 with HEPES buffer (0.02 mol L⁻¹, pH 7.5, I=0.1 mol L⁻¹ with NaCl).

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

What is claimed is:

1. A compound of Formula (I) or a pharmaceutically acceptable salt, prodrug or prodrug ester: 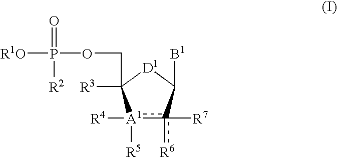
wherein:
each [symbol] is a double or single bond;
A¹ is selected from the group consisting of C (carbon), O (oxygen) and S (sulfur);
B¹ is an optionally substituted heterocyclic base or a derivative thereof;
D¹ is selected from the group consisting of C═CH₂, CH₂, O (oxygen) and S (sulfur);
R¹ is 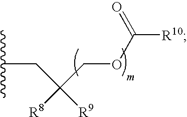
R² is an —N-linked amino acid;
R³ is selected from the group consisting of hydrogen, azido, —CN, an optionally substituted C₁₋₄ alkyl and an optionally substituted C₁₋₄ alkoxy;
R⁴ is absent or selected from the group consisting of hydrogen, halogen, hydroxy and an optionally substituted C₁₋₄ alkyl;
R⁵ is absent or selected from the group consisting of hydrogen, halogen, azido, amino, hydroxy, and an —O-linked amino acid;
R⁶ is absent or selected from the group consisting of hydrogen, halogen, hydroxy, —CN, —NC, an optionally substituted C₁₋₄ alkyl, an optionally substituted C₁₋₄ alkoxy and an —O-linked amino acid;
R⁷ is absent or selected from the group consisting of hydrogen, halogen, hydroxy, —CN, —NC, an optionally substituted C₁₋₄ alkyl, an optionally substituted haloalkyl and an optionally substituted hydroxyalkyl, or when the bond to R⁶ indicated by [symbol] is a double bond, then R⁶ a C₁₋₄ alkenyl and R⁷ is absent;
R⁸ and R⁹ are each independently —C≡N or an optionally substituted substituent selected from the group consisting of C₁₋₈ organylcarbonyl, C₁₋₈ alkoxycarbonyl and C₁₋₈ organylaminocarbonyl;
R¹⁰ is hydrogen or an optionally substituted C₁₋₄-alkyl; and
m is 1 or 2.

2. The compound of claim 1, wherein A¹ is C (carbon), D¹ is O (oxygen), and both bonds indicated by [symbol] are single bonds.

3. The compound of claim 1, wherein R⁸ is —C≡N, and R⁹ is an optionally substituted C₁₋₈ alkoxycarbonyl or an optionally substituted C₁₋₈ organylaminocarbonyl.

4. The compound of claim 1, wherein both R⁸ and R⁹ are an optionally substituted C₁₋₈ organylcarbonyl or an optionally substituted C₁₋₈ alkoxycarbonyl.

5. The compound of claim 1, wherein m is 2; both R⁸ and R⁹ are an optionally substituted C₁₋₈ organylcarbonyl; and R¹⁰ is an optionally substituted C₁₋₄-alkyl.

6. The compound of claim 1, wherein 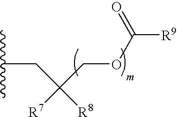
is selected from the group consisting of:

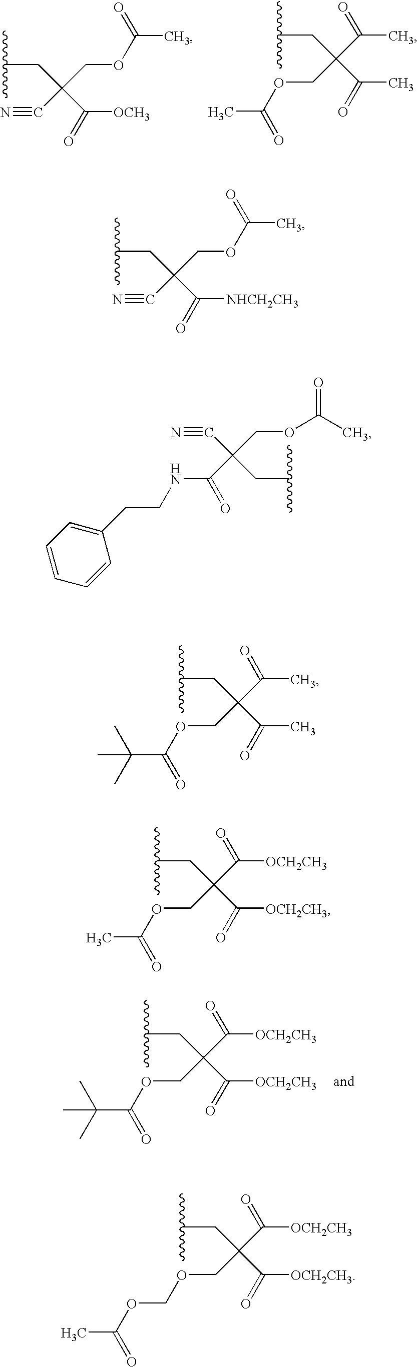

7. The compound of claim 1, wherein R² is: 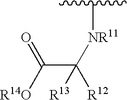
R¹¹ is hydrogen or an optionally substituted C₁₋₄-alkyl;
R¹² is selected from the group consisting of hydrogen, an optionally substituted C₁₋₆-alkyl, an optionally substituted aryl, an optionally substituted aryl(C₁₋₄ alkyl) and haloalkyl;
R¹³ is hydrogen or an optionally substituted C₁₋₄-alkyl; and
R¹⁴ is selected from the group consisting of an optionally substituted C₁₋₆ alkyl, an optionally substituted C₆ aryl, an optionally substituted C₁₀ aryl, and an optionally substituted C₃₋₆ cycloalkyl.

8. The compound of claim 7, wherein R¹¹ is hydrogen and R¹⁴ is an optionally substituted C₁₋₆ alkyl.

9. The compound of claim 7, wherein R² is:

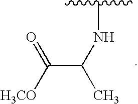

10. The compound of claim 1, wherein at least one of R⁵ and R⁶ is hydroxyl or an —O-linked amino acid.

11. The compound claim 10, wherein the —O-linked amino acid is selected from the group consisting of alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine.

12. The compound claim 10, wherein the —O-linked amino acid is selected from the group consisting of —O-linked α-amino acid, —O-linked β-amino acid, —O-linked γ-amino acid and —O-linked δ-amino acid.

13. The compound of claim 1, wherein B¹ is selected from the group consisting of: 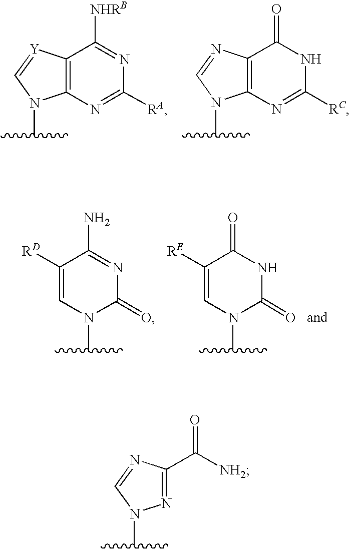
wherein:
R^{A} is hydrogen or halogen;
R^{B} is hydrogen, an optionally substituted C₁₋₄ alkyl, or an optionally substituted C₃₋₈ cycloalkyl;
R^{C} is hydrogen or amino;
R^{D} is hydrogen or halogen;
R^{E} is hydrogen or an optionally substituted C₁₋₄alkyl; and
Y is N or CR^{F}, wherein R^{F} can be selected from the group consisting of hydrogen, halogen, an optionally substituted C₁₋₄-alkyl, an optionally substituted C₂₋₄-alkenyl and an optionally substituted C₂₋₄-alkynyl.

14. A compound of Formula (II) or a pharmaceutically acceptable salt, prodrug or prodrug ester: 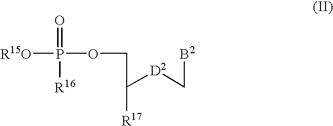
wherein:
B² is an optionally substituted heterocyclic base or an optionally substituted heterocyclic base derivative thereof;
D² is selected from the group consisting of C═CH₂, CH₂, O (oxygen) and S (sulfur);
R¹⁵ is 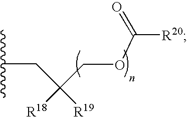
R¹⁶ is an —N-linked amino acid;
R¹⁷ is hydrogen or —(CH₂)—OH;
R¹⁸ and R¹⁹ are each independently —C≡N or an optionally substituted substituent selected from C₁₋₈ organylcarbonyl, C₁₋₈ alkoxycarbonyl and C₁₋₈ organylaminocarbonyl;
R²⁰ is hydrogen or an optionally substituted C₁₋₄-alkyl; and
n can be 1 or 2.

15. The compound of claim 14, wherein D² is O (oxygen).

16. The compound of claim 14, wherein R¹⁸ is —C≡N, and R¹⁹ is an optionally substituted C₁₋₈ alkoxycarbonyl or an optionally substituted C₁₋₈ organylaminocarbonyl.

17. The compound of claim 14, wherein both R¹⁸ and R¹⁹ are an optionally substituted C₁₋₈ organylcarbonyl or an optionally substituted C₁₋₈ alkoxycarbonyl.

18. The compound of claim 14, wherein n is 2; both R¹⁸ and R¹⁹ are an optionally substituted C₁₋₈ organylcarbonyl; and R²⁰ is an optionally substituted C₁₋₄-alkyl.

19. The compound of claim 14, wherein 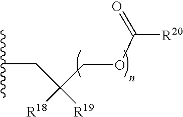
is selected from the group consisting of:

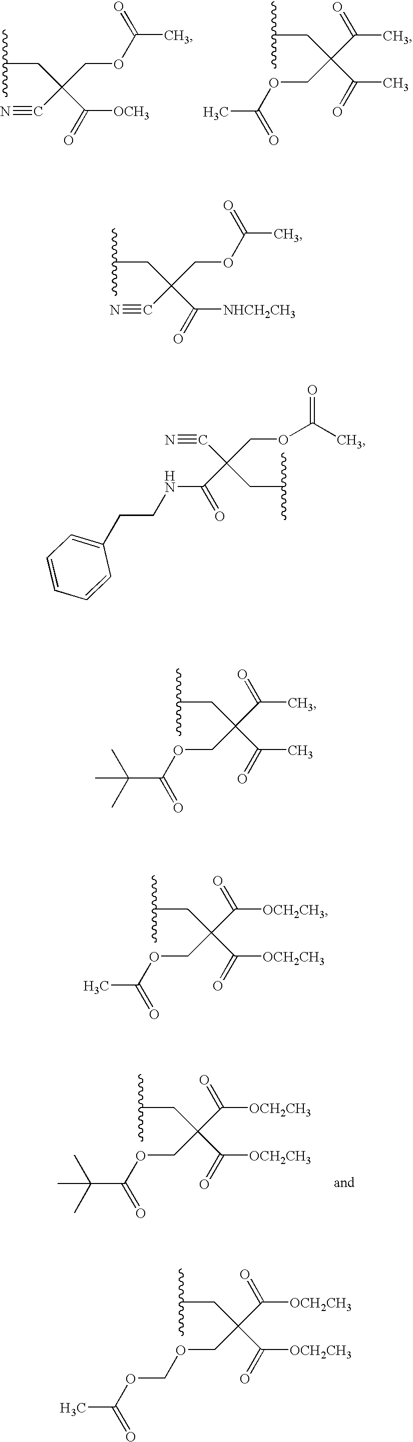

20. The compound of claim 14, wherein R¹⁶ is: 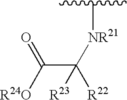
wherein:
R²¹ is hydrogen or an optionally substituted C₁₋₄-alkyl;
R²² is selected from the group consisting of hydrogen, an optionally substituted C₁₋₆-alkyl, an optionally substituted aryl, an optionally substituted aryl(C₁₋₄ alkyl) and haloalkyl;
R²³ is hydrogen or an optionally substituted C₁₋₄-alkyl; and
R²⁴ is selected from the group consisting of an optionally substituted C₁₋₆ alkyl, an optionally substituted C₆ aryl, an optionally substituted C₁₀ aryl, and an optionally substituted C₃₋₆ cycloalkyl.

21. The compound of claim 20, wherein R²¹ is hydrogen and R²⁴ is an optionally substituted C₁₋₆ alkyl.

22. The compound of claim 20, wherein R¹⁶ is:

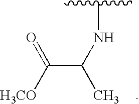

23. The compound of claim 14, wherein B² is selected from the group consisting of: 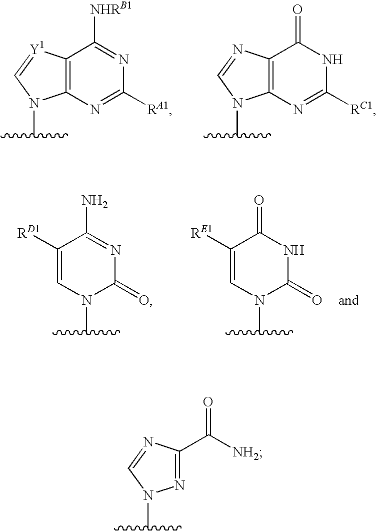
wherein:
R^{A1} is hydrogen or halogen;
R^{B1} is hydrogen, an optionally substituted C₁₋₄ alkyl, or an optionally substituted C₃₋₈ cycloalkyl;
R^{C1} is hydrogen or amino;
R^{D1} is hydrogen or halogen;
R^{E1} is hydrogen or an optionally substituted C₁₋₄alkyl; and
Y¹ is N or CR^{F1}, wherein R^{F1} can be selected from the group consisting of hydrogen, halogen, an optionally substituted C₁₋₄-alkyl, an optionally substituted C₂₋₄-alkenyl and an optionally substituted C₂₋₄-alkynyl.

24. A compound of Formula (III) or a pharmaceutically acceptable salt, prodrug or prodrug ester: 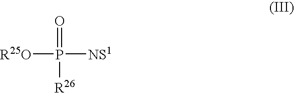
wherein: NS¹ is a nucleoside attached to the phosphorus via the oxygen bonded to the 5′-carbon;
R²⁵ is 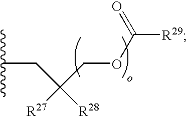
R²⁶ is an —N-linked amino acid;
R²⁷ and R²⁸ are each independently —C≡N or an optionally substituted substituent selected from the group consisting of C₁₋₈ organylcarbonyl, C₁₋₈ alkoxycarbonyl and C₁₋₈ organylaminocarbonyl;
R²⁹ is hydrogen or an optionally substituted C₁₋₄-alkyl; and
o is 1 or 2.

25. The compound of claim 24, wherein R²⁷ is —C1\1, and R²⁸ is an optionally substituted C₁₋₈ alkoxycarbonyl or an optionally substituted C₁₋₈ organylaminocarbonyl.

26. The compound of claim 24, wherein both R²⁷ and R²⁸ are an optionally substituted C₁₋₈ organylcarbonyl or an optionally substituted C₁₋₈ alkoxycarbonyl.

27. The compound of claim 24, wherein o is 2; both R²⁷ and R²⁸ are an optionally substituted C₁₋₈ organylcarbonyl; and R²⁹ is an optionally substituted C₁₋₄-alkyl.

28. The compound of claim 24, wherein 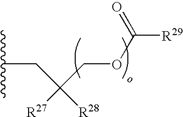
is selected from the group consisting of:

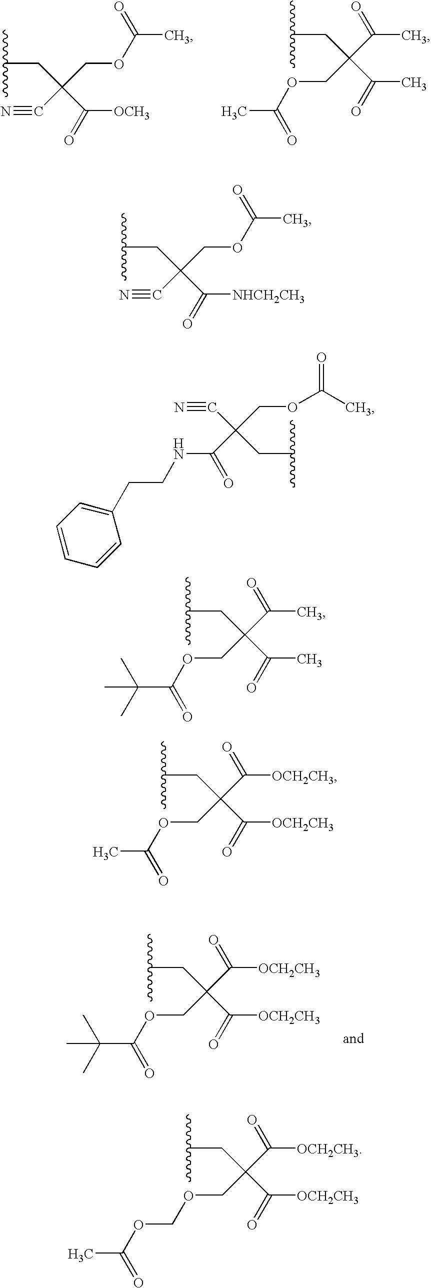

29. The compound of claim 24, wherein R²⁶ is: 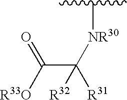
wherein:
R³⁰ is hydrogen or an optionally substituted C₁₋₄-alkyl;
R³¹ is selected from the group consisting of hydrogen, an optionally substituted C₁₋₆-alkyl, an optionally substituted aryl, an optionally substituted aryl(C₁₋₄ alkyl) and haloalkyl;
R³² is hydrogen or an optionally substituted C₁₋₄-alkyl; and
R³³ is selected from the group consisting of an optionally substituted C₁₋₆ alkyl, an optionally substituted C₆ aryl, an optionally substituted C₁₀ aryl, and an optionally substituted C₃₋₆ cycloalkyl.

30. The compound of claim 29, wherein R³⁰ is hydrogen and R³³ is an optionally substituted C₁₋₆ alkyl.

31. The compound of claim 29, wherein R²⁶ is:

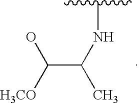

32. The compound of claim 24, wherein NS¹ has the structure: 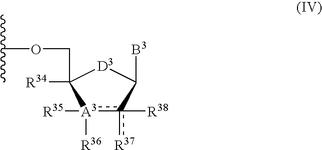
wherein:
each [symbol] is a double or single bond;
A³ is selected from the group consisting of C (carbon), O (oxygen) and S (sulfur);
B³ is an optionally substituted heterocyclic base or a derivative thereof;
D³ is selected from the group consisting of C═CH₂, CH₂, O (oxygen) and S (sulfur);
R³⁴ is selected from the group consisting of hydrogen, azido, —CN, an optionally substituted C₁₋₄ alkyl and an optionally substituted C₁₋₄ alkoxy;
R³⁵ is absent or selected from the group consisting of hydrogen, halogen, hydroxy and an optionally substituted C₁₋₄ alkyl;
R³⁶ is absent or selected from the group consisting of hydrogen, halogen, azido, amino, hydroxy and an —O-linked amino acid;
R³⁷ is selected from the group consisting of hydrogen, halogen, hydroxy, —CN, —NC, an optionally substituted C₁₋₄ alkyl, an optionally substituted C₁₋₄ alkoxy and an —O-linked amino acid; and
R³⁸ is absent or selected from the group consisting of hydrogen, halogen, hydroxy, —CN, —NC, an optionally substituted C₁₋₄ alkyl, an optionally substituted haloalkyl and an optionally substituted hydroxyalkyl, or when the bond to R³⁷ indicated by [symbol] is a double bond, then R³⁷ is a C₁₋₄ alkenyl and R³⁸ is absent.

33. The compound of claim 32, wherein A³ is C (carbon), D³ is O (oxygen), and both bonds indicated by [symbol] are single bonds.

34. The compound of claim 32, wherein at least one of R³⁶ and R³⁷ is hydroxyl or an —O-linked amino acid.

35. The compound claim 34, wherein the —O-linked amino acid is selected from the group consisting of alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine.

36. The compound claim 34, wherein the —O-linked amino acid is selected from the group consisting of —O-linked α-amino acid, —O-linked β-amino acid, —O-linked γ-amino acid and —O-linked δ-amino acid.

37. The compound of claim 32, wherein B³ is selected from the group consisting of: 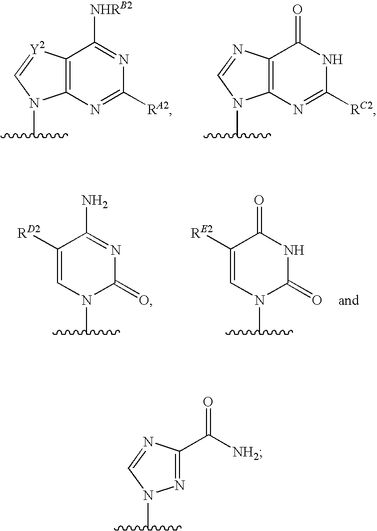
wherein:
R^{A2} is hydrogen or halogen;
R^{B2} is hydrogen, an optionally substituted C₁₋₄ alkyl, or an optionally substituted C₃₋₈ cycloalkyl;
R^{C2} is hydrogen or amino;
R^{D2} is hydrogen or halogen;
R^{E2} is hydrogen or an optionally substituted C₁₋₄alkyl; and
Y² is N or CR^{F2}, wherein R^{F2} can be selected from the group consisting of hydrogen, halogen, an optionally substituted C₁₋₄-alkyl, an optionally substituted C₂₋₄-alkenyl and an optionally substituted C₂₋₄-alkynyl.

38. The compound of claim 24, wherein NS¹ has the structure: 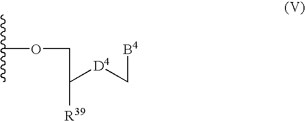
wherein:
B⁴ is an optionally substituted heterocyclic base or a derivative thereof;
D⁴ is selected from the group consisting of C═CH₂, CH₂, O (oxygen) and S (sulfur); and
R³⁹ is hydrogen or —(CH₂)—OH.

39. The compound of claim 38, wherein D² is O (oxygen).

40. The compound of claim 38, wherein B⁴ is selected from the group consisting of: 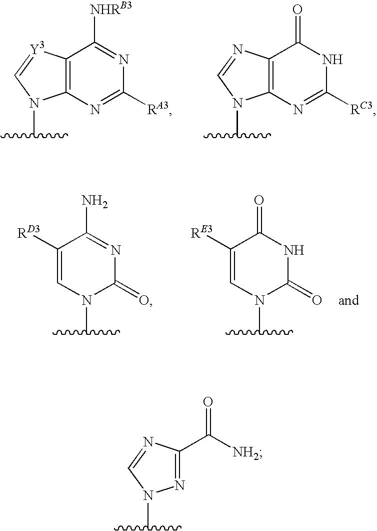
wherein:
R^{A3} is hydrogen or halogen;
R^{B3} is hydrogen, an optionally substituted C₁₋₄ alkyl, or an optionally substituted C₃₋₈ cycloalkyl;
R^{C3} is hydrogen or amino;
R^{D3} is hydrogen or halogen;
R^{E3} is hydrogen or an optionally substituted C₁₋₄alkyl; and
Y³ is N or CR^{F3}, wherein R^{F3} can be selected from the group consisting of hydrogen, halogen, an optionally substituted C₁₋₄-alkyl, an optionally substituted C₂₋₄-alkenyl and an optionally substituted C₂₋₄-alkynyl.

41. The compound of claim 1, wherein the compound is selected from the group consisting of:

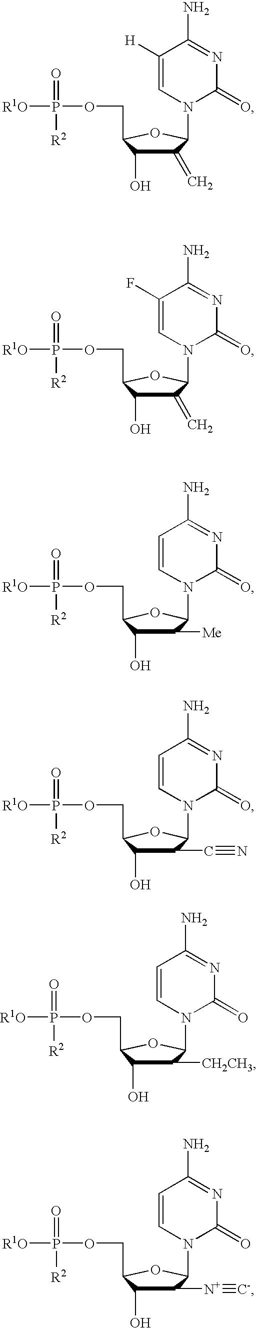

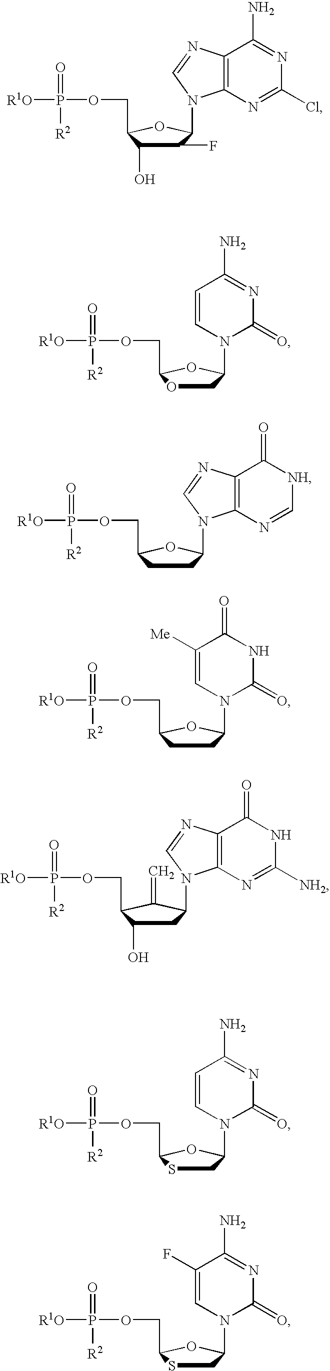

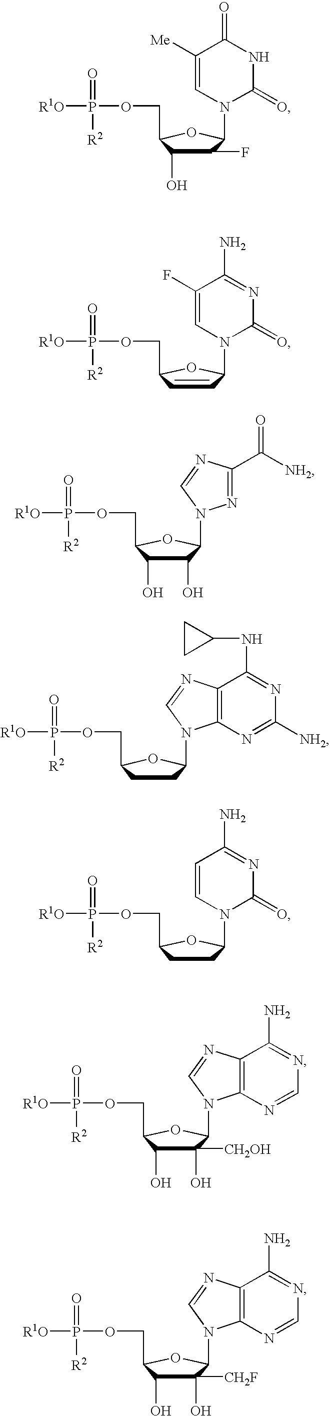

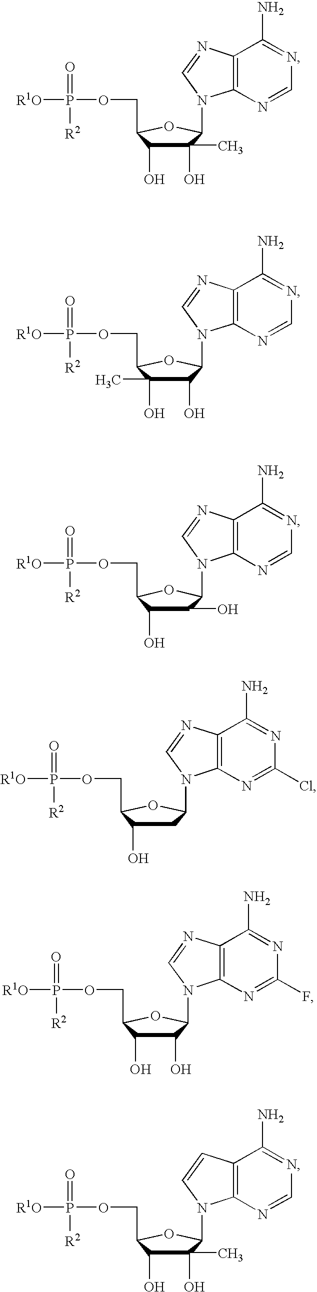

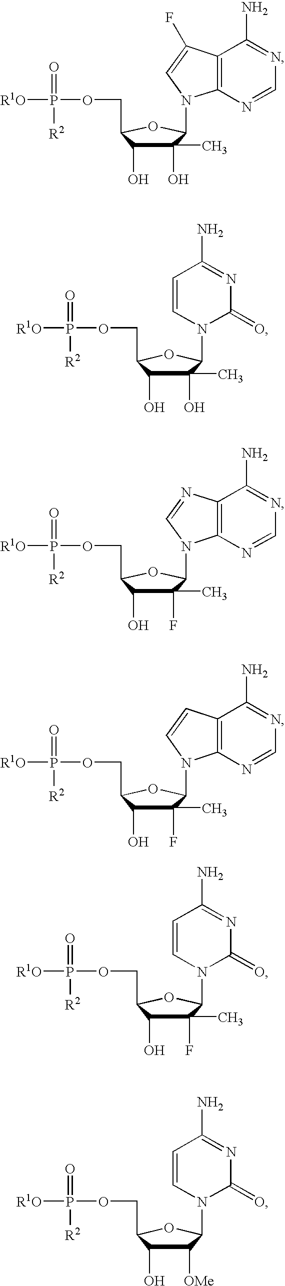

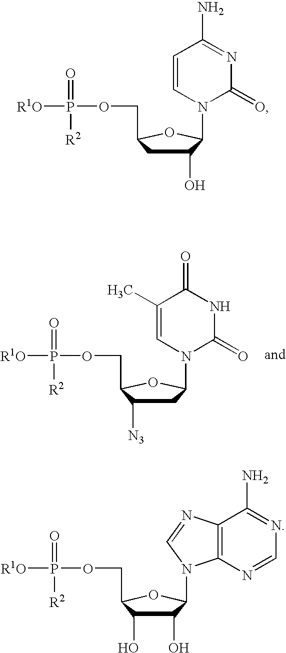

42. The compound of claim 14, wherein the compound is selected from the group consisting of:

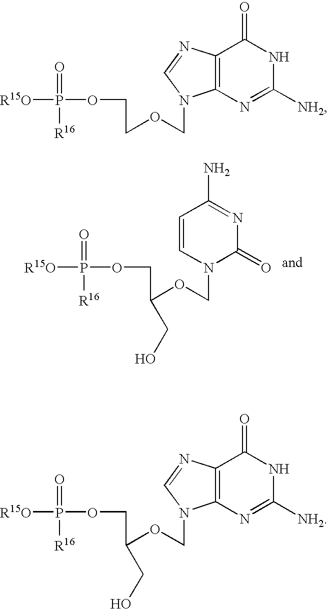

43. The compound of claim 1, wherein the compound of Formula (I) is selected from the group consisting of:

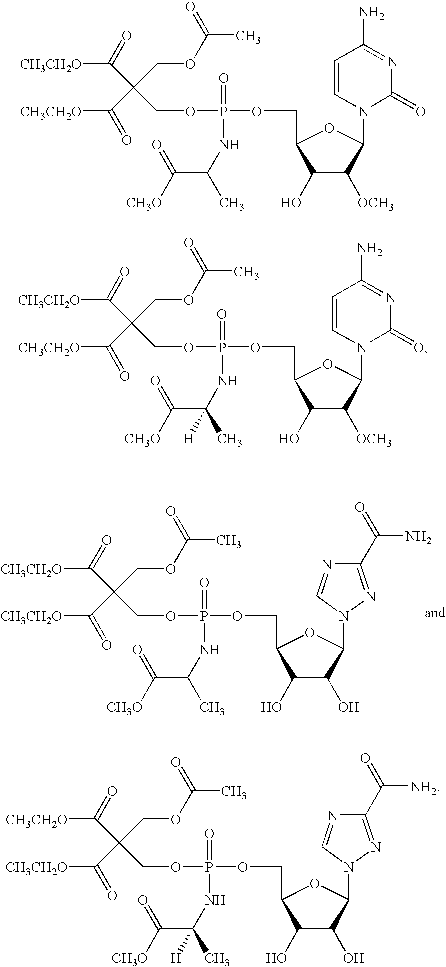

44. A pharmaceutical composition comprising a compound of claim 1, and a pharmaceutically acceptable carrier, diluent, excipient or combination thereof.

45. A method of ameliorating or treating a neoplastic disease comprising administering to a subject suffering from the neoplastic disease a therapeutically effective amount of a compound of claim 1.

46. The method of claim 45, wherein the neoplastic disease is cancer.

47. The method of claim 45, wherein the neoplastic disease is leukemia.

48. A method of ameliorating or treating a viral infection comprising administering to a subject suffering from the viral infection a therapeutically effective amount of a compound of claim 1.

49. The method of claim 48, wherein the viral infection is caused by a virus selected from the group consisting of an adenovirus, an Alphaviridae, an Arbovirus, an Astrovirus, a Bunyaviridae, a Coronaviridae, a Filoviridae, a Flaviviridae, a Hepadnaviridae, a Herpesviridae, an Alphaherpesvirinae, a Betaherpesvirinae, a Gammaherpesvirinae, a Norwalk Virus, an Astroviridae, a Caliciviridae, an Orthomyxoviridae, a Paramyxoviridae, a Paramyxoviruses, a Rubulavirus, a Morbillivirus, a Papovaviridae, a Parvoviridae, a Picornaviridae, an Aphthoviridae, a Cardioviridae, an Enteroviridae, a Coxsackie virus, a Polio Virus, a Rhinoviridae, a Phycodnaviridae, a Poxyiridae, a Reoviridae, a Rotavirus, a Retroviridae, an A-Type Retrovirus, an Immunodeficiency Virus, a Leukemia Viruses, an Avian Sarcoma Viruses, a Rhabdoviruses, a Rubiviridae and a Togaviridae.

50. The method of claim 48, wherein the viral infection is a hepatitis C viral infection, or a hepatitis B viral infection, or a HIV viral infection.

51. A method of ameliorating or treating a parasitic disease comprising administering to a subject suffering from the parasitic disease a therapeutically effective amount of a compound of claim 1.

52. The method of claim 51, wherein the parasitic disease is Chagas' disease.
